(19) United States
(12) Patent Application Publication
(10) Pub. No.: US 2018/0114600 A1
(43) Pub. Date: Apr. 26, 2018

(54) METHODS AND SYSTEMS FOR DETERMINING RISK OF A PREGNANCY COMPLICATION OCCURRING

(71) Applicant: Adelaide Research & Innovation Pty Ltd, Adelaide, South Australia (AU)

(72) Inventors: Claire Trelford Roberts, Hawthorndene, South Australia (AU); Shalem Yiner-Lee Leemaqz, Valley View, South Australia (AU); Gustaaf Albert Dekker, Norton Summit, South Australia (AU)

(21) Appl. No.: 15/560,710

(22) PCT Filed: Mar. 23, 2016
(86) PCT No.: PCT/AU2016/050212

§ 371 Date: Sep. 22, 2017

(30) Foreign Application Priority Data
Mar. 23, 2015 (AU) 2015901036

(51) Int. Cl. G16H 50/30; G06F 19/24; A61K 31/616; A61K 31/57; A61K 31/155; G06F 19/22

(52) U.S. Cl. CPC G16H 50/30; G06F 19/24; G06F 19/22; A61K 31/57; A61K 31/155; A61K 31/616

(57) ABSTRACT

The present disclosure relates to methods and systems for determining the risk of a complication of pregnancy occurring. Certain embodiments of the present disclosure provide a method of determining the risk of a complication of pregnancy occurring in a subject, the pregnancy arising in the subject from a conception from a maternal donor and a paternal donor. The method comprises receiving initial information comprising (i) initial genetic information and/or (ii) initial clinical information and/or (iii) initial lifestyle information, the initial information being from the subject and/or the maternal donor and/or the paternal donor; processing the initial information to classify the risk of a complication of pregnancy occurring in the subject as low risk or increased risk; for the subject having said increased risk, receiving further information, the further information comprising (i) further genetic information and/or (ii) further clinical information and/or (iii) further lifestyle information from the subject and/or the maternal donor and/or the paternal donor; processing the further information to classify the risk in the subject having said increased risk as moderate risk or high risk, thereby determining the risk of a complication of pregnancy occurring in the subject.

### PRIORITY CLAIM

This application claims priority to Australian provisional patent application number 2015901036 filed on 23 Mar. 2015, the content of which is hereby incorporated by reference.

### FIELD

The present disclosure relates to methods and systems for determining the risk of a complication of pregnancy occurring.

### BACKGROUND

A significant degree of maternal and fetal morbidity and mortality result from complications of pregnancy. Of these, the major complications of preeclampsia, preterm birth, small for gestational age, intrauterine growth restriction (IUGR) and gestational diabetes mellitus occur in about 25% of first pregnancies. In more than 6% of pregnancies, these complications are life-threatening to either, or both, of the mother and her baby during gestation and may also affect neonatal health and survival post-delivery. Furthermore, multiple complications may also occur in a single pregnancy.

Currently there are no effective methods of predicting these complications in first time mothers, and by the time symptoms of the conditions are present it is often too late for effective treatment.

One example of a complication of pregnancy is preeclampsia. Preeclampsia is a condition characterized by hypertension during pregnancy and elevated levels of protein in urine. Preeclampsia is commonly diagnosed by two separate blood pressure readings taken at least 6 hours apart of 140/90 mm Hg or the presence of at least 300 mg of protein in a 24-hour urine sample. Preeclampsia may also be associated with sudden swelling and rapid gain of weight. Preeclampsia is the most common of life-threatening complications during pregnancy for both the mother and the preterm baby.

Preeclampsia occurs in up to 10% of pregnancies and is most prevalent in first pregnancies. Preeclampsia onset typically occurs after 20 weeks gestation and continues throughout pregnancy. Symptoms associated with preeclampsia may also occur or persist up to eight weeks after delivery of the baby. Preeclampsia is associated with increased morbidity and mortality for the mother and the baby, and can also lead to the development of eclampsia which necessitates the mother being treated in intensive care. About 10% of cases of preeclampsia are said to be early-onset, which is characterized as being diagnosed before 34 weeks gestation.

Another example of a complication of pregnancy is preterm birth. Preterm birth (PTB) is a birth before 37 weeks gestation and occurs in 8-12% of pregnancies. PTB may be spontaneous (SPTB) or induced, and may be the consequence of complications such as premature rupture of membranes or preeclampsia. Many babies born close to full term will live healthy and normal lives. However, the chances of morbidity and mortality dramatically increase as the level of prematurity of birth increases, that is as gestational age decreases. About 75% of PTB is said to be late preterm occurring between 34 and 37 weeks gestation, about 20-25% of PTB occurs at 30-33 weeks gestation and the remaining 5-10% occur at 24-29 weeks gestation. The latter group is most likely to die or suffer long term health problems such as cerebral palsy, vision impairment and lung disease. However, even babies born late preterm can have long term health or learning difficulties.

Intrauterine growth restriction (IUGR) is a condition in which growth of the fetus is restricted. Intrauterine growth restriction typically results in a fetus which is small for its gestational age (SGA). IUGR and SGA occur in approximately 5-10% of pregnancies. Intrauterine growth restriction may occur at any stage in pregnancy, and may result in a full term or preterm delivery. Regardless of the duration of pregnancy, IUGR typically results in a fetus of a weight less than the tenth percentile. Some of these babies may be small for constitutional reasons, that is they are genetically destined to be small whereas others are growth restricted. An IUGR baby is one who is SGA and at birth weighs less than the fifth centile. An IUGR infant may also be said to be of low birth weight (<2500 g). IUGR may occur as a result of poor health of the mother, poor nutrition, decreased blood flow to the uterus and placenta, preeclampsia or an infection in the tissues around the fetus. While it is not possible to reverse the effects of IUGR, treatments such as improving maternal nutrition, bed rest or early delivery may minimize the effects on the fetus.

Gestational diabetes mellitus (GDM) occurs when a pregnant woman becomes diabetic during pregnancy without having been so prior to pregnancy. Its presence is usually tested for at about 28 weeks gestation and sometimes first by an oral glucose challenge test, and if positive it is definitively diagnosed by an oral glucose tolerance test. Sometimes an oral glucose tolerance test is the first test performed. Once diagnosed, women who develop GDM are given insulin, insulin sensitizing drugs or undertake diet and exercise interventions. If uncontrolled, GDM in the mother results in high glucose transport across the placenta into the fetal circulation, elevating fetal insulin (a growth factor for the fetus) levels and increasing the glucose availability, resulting in increased growth. Consequently, these babies are likely to be large for gestational age (LGA) at birth and may require caesarean section for delivery. However, when caesarean sections are not available there is an elevated risk that both the mother and the baby may suffer a birth injury. These babies are also more likely to require care in a neonatal nursery, in comparison to babies having weight appropriate for gestational age. In the longer term, women who had GDM are also more likely to develop type 2 diabetes within 5-10 years of the birth of their child.

A variety of antenatal intervention or management strategies can be employed for subjects considered to be at risk for the above complications of pregnancy. For example, management strategies such as increased monitoring and bed rest or treatment with low dose aspirin may be adopted for patients considered to be at risk of suffering from preeclampsia or intrauterine growth restriction. Subjects considered to be at risk of suffering from preterm birth may use a number of lifestyle changes or may be treated with vaginal progesterone. Subjects deemed to be at risk of suffering from gestational diabetes may be subject to an early oral glucose tolerance test, increased monitoring and adopt a number of lifestyle changes or be treated with diabetes medicine or insulin if deemed appropriate.

Given the associated mortality and short-term and long-term morbidity associated with complications of pregnancy, there is a need for methods of determining the risks of such complications occurring.

### SUMMARY

The present disclosure relates to methods and systems for determining the risk of a complication of pregnancy occurring.

Certain embodiments of the present disclosure provide a method of determining the risk of a complication of pregnancy occurring in a subject, the pregnancy arising in the subject from a conception from a maternal donor and a paternal donor, the method comprising:
receiving initial information comprising (i) initial genetic information and/or (ii) initial clinical information and/or (iii) initial lifestyle information, the initial information being from the subject and/or the maternal donor and/or the paternal donor;
processing the initial information to classify the risk of a complication of pregnancy occurring in the subject as low risk or increased risk;
for the subject having said increased risk, receiving further information, the further information comprising (i) further genetic information and/or (ii) further clinical information and/or (iii) further lifestyle information from the subject and/or the maternal donor and/or the paternal donor;
processing the further information to classify the risk in the subject having said increased risk as moderate risk or high risk,
thereby determining the risk of a complication of pregnancy occurring in the subject.

Certain embodiments of the present disclosure provide a method of determining the risk of a complication of pregnancy occurring in a subject, the pregnancy arising in the subject from a conception from a maternal donor and a paternal donor, the method comprising:
initially classifying the risk in the subject as a low risk or an increased risk; and
further classifying the risk in the subject at increased risk as at a moderate risk or at a high risk;
thereby determining whether the risk of a complication of pregnancy occurring in the subject is a low risk, a moderate risk or a high risk.

Certain embodiments of the present disclosure provide a method of determining the risk of a complication of pregnancy occurring in a subject, the pregnancy arising in the subject from a conception from a maternal donor and a paternal donor, the method comprising:
initially classifying the risk in the subject as a low risk or an increased risk based on initial information comprising (i) initial genetic information and/or (ii) initial clinical information and/or (iii) initial lifestyle information, the initial information being from the subject and/or the maternal donor and/or the paternal donor, and
further classifying the risk in the subject having said increased risk as a moderate risk or a high risk based on information comprising (i) further genetic information and/or (ii) further clinical information and/or (iii) further lifestyle information, the further information being from the subject and/or the maternal donor and/or the paternal donor,
thereby determining whether the risk of a complication of pregnancy occurring in the subject is a low risk, a moderate risk or a high risk.

Certain embodiments of the present disclosure provide a method of determining the risk of a complication of pregnancy occurring in a subject, the pregnancy arising in the subject from a conception from a maternal donor and a paternal donor, the method comprising using a computer processor means to:
receive initial information comprising (i) initial genetic information and/or (ii) initial clinical information and/or (iii) initial lifestyle information, the initial information being from the subject and/or the maternal donor and/or the paternal donor, and process the initial information to classify the risk of a complication of pregnancy occurring in the subject as low risk or increased risk;
for the subject having said increased risk, receive further information, the further information comprising (i) further genetic information and/or (ii) further clinical information and/or (iii) further lifestyle information from the subject and/or the maternal donor and/or the paternal donor; and
process the further information to classify the risk in the subject having said increased risk as moderate risk or high risk; and
output the risk of the complication of pregnancy occurring in the subject.

Certain embodiments of the present disclosure provide a system for determining the risk of a complication of pregnancy occurring in a subject, the pregnancy arising in the subject from a conception from a maternal donor and a paternal donor, the system comprising a computer processor configured to:
receive initial information from at least one user device in data communication with the processor over a network, the initial information comprising (i) initial genetic information and/or (ii) initial clinical information and/or (iii) initial lifestyle information, the initial information being from the subject and/or maternal donor and/or the paternal donor, and
process the initial information to classify the risk of a complication of pregnancy occurring in the subject as low risk or increased risk;
for the subject having said increased risk, the processor is further configured to receive further information, from the at least one user device or a further user device in data communication with the processor, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk; and
output the risk of the complication of pregnancy occurring in the subject.

Certain embodiments of the present disclosure provide a computer-readable medium encoded with programming instructions executable by a computer processor means to allow the computer processor means to determine the risk of a complication of pregnancy occurring in a subject, the pregnancy arising in the subject from a conception from a maternal donor and a paternal donor, wherein the instructions allow the computer processing means to:
receive initial information comprising (i) initial genetic information and/or (ii) initial clinical information and/or (iii) initial lifestyle information, the initial information being from the subject and/or the maternal donor and/or the paternal donor, and
process the initial information to classify the risk of a complication of pregnancy occurring in the subject as low risk or increased risk; and
for the subject having said increased risk, receive further information, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk.

Certain embodiments of the present disclosure provide computer software encoded with programming instructions executable by a computer processor means to allow the computer processor means to determine the risk of a complication of pregnancy occurring in a subject, the pregnancy arising in the subject from a conception from a maternal donor and a paternal donor, wherein the software allows the computer processing means to:
receive initial information comprising (i) initial genetic information and/or (ii) initial clinical information and/or (iii) initial lifestyle information, the initial information being from the subject and/or the maternal donor and/or the paternal donor, and
process the initial information to classify the risk of a complication of pregnancy occurring in the subject as low risk or increased risk; and
for the subject having said increased risk, receive further information, the further information comprising (i) further genetic information and/or (ii) further clinical information and/or (iii) further lifestyle information, the further information being from the subject and/or the maternal donor and/or the paternal donor, and process the further information to classify the risk in the subject having said increased risk as moderate risk or high risk.

Certain embodiments of the present disclosure provide a method of preventing and/or treating a complication of pregnancy in a subject, the method comprising using a method as described herein to determine the risk of a complication of a pregnancy occurring and treating the subject on the basis of the risk so determined.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows tiered prediction system with potential variables in the models for each tier.

FIG. 2 shows the characteristics of Penalized Logistic regression used to develop individual models (top), and model integration process for final classification (below).

FIG. 3 shows the definition of accuracy measures used to assess models.

FIG. 4 shows an example of PE model in Tier 1 aiming at a higher sensitivity.

FIG. 5 shows an example of PE model in Tier 2 aiming at a higher positive predictive value (PPV).

FIG. 6 shows the overall workflow from data mining the raw database, to model development, and to final risk classification for tailored antenatal care.

FIG. 7 shows the workflow for individual models.

FIG. 8 shows the workflow for model integration.

FIG. 9 shows PE model (tier 2) variable shrinkage pathway.

FIG. 10 shows SPTB model (tier 1) variable shrinkage pathway.

FIG. 11 shows SPTB model (tier 2) variable shrinkage pathway.

FIG. 12 shows SGA model (tier 1) variable shrinkage pathway.

FIG. 13 shows SGA model (tier 2) variable shrinkage pathway.

FIG. 14 shows GDM model (tier 1) variable shrinkage pathway.

FIG. 15 shows GDM model (tier 2) variable shrinkage pathway.

FIG. 16 shows tiered model specifications.

FIG. 17 shows model integration.

FIG. 18 shows tiered model risk classification.

FIG. 19 shows final risk classification for PE (Panel A), SPTB (Panel B), SGA (Panel C), and GDM (Panel D).

### DETAILED DESCRIPTION

The present disclosure relates to methods and systems for determining the risk of a pregnancy complication occurring.

The present disclosure is based on the recognition that the risk of suffering a complication of pregnancy may be determined using a two-tiered approach to provide three tiers of risk: low, moderate and high risk.

In certain embodiments, the present disclosure provides a suite of methods (and algorithms with or without accompanying software) to predict early in pregnancy the subsequent risk of the main late pregnancy complications: preeclampsia, preterm birth, intrauterine growth restriction (IUGR), small for gestational age and gestational diabetes mellitus. The methods (and algorithms) are based on genetic information (eg single nucleotide polymorphisms (SNPs)) in the mother and/or father, and clinical and/or lifestyle variables. Each prediction model takes a two-tiered approach with two independent methods/algorithms that are integrated to provide three tiers of risk: low, moderate and high risk.

Certain embodiments of the present disclosure provide a method of determining the risk of a complication of pregnancy occurring in a subject.

Certain embodiments of the present disclosure provide a method of determining the risk of a complication of pregnancy occurring in a subject, the pregnancy arising in the subject from a conception from a maternal donor and a paternal donor, the method comprising:
receiving initial information comprising (i) initial genetic information and/or (ii) initial clinical information and/or (iii) initial lifestyle information, the initial information being from the subject and/or the maternal donor and/or the paternal donor;
processing the initial information to classify the risk of a complication of pregnancy occurring in the subject as low risk or increased risk;
for the subject having said increased risk, receiving further information, the further information comprising (i) further genetic information and/or (ii) further clinical information and/or (iii) further lifestyle information from the subject and/or the maternal donor and/or the paternal donor;
processing the further information to classify the risk in the subject having said increased risk as moderate risk or high risk,
thereby determining the risk of a complication of pregnancy occurring in the subject.

Certain embodiments of the present disclosure provide a method of determining the risk of a complication of pregnancy occurring in a subject, the pregnancy arising in the subject from a conception from a maternal donor and a paternal donor, the method comprising:
initially classifying the risk in the subject as a low risk or an increased risk; and
further classifying the risk in the subject at increased risk as at a moderate risk or at a high risk;
thereby determining whether the risk of a complication of pregnancy occurring in the subject is a low risk, a moderate risk or a high risk.

Certain embodiments of the present disclosure provide a method of determining the risk of a complication of pregnancy occurring in a subject, the pregnancy arising in the subject from a conception from a maternal donor and a paternal donor, the method comprising:
initially classifying the risk in the subject as a low risk or an increased risk based on initial information comprising (i) initial genetic information and/or (ii) initial clinical information and/or (iii) initial lifestyle information, the initial information being from the subject and/or the maternal donor and/or the paternal donor, and
further classifying the risk in the subject having said increased risk as a moderate risk or a high risk based on information comprising (i) further genetic information and/or (ii) further clinical information and/or (iii) further lifestyle information, the further information being from the subject and/or the maternal donor and/or the paternal donor,
thereby determining whether the risk of a complication of pregnancy occurring in the subject is a low risk, a moderate risk or a high risk.

In certain embodiments, the methods of the present disclosure are used to determine the likelihood of a complication of pregnancy occurring in the subject.

In certain embodiments, the complication of pregnancy comprises one or more of preeclampsia, preterm birth, small for gestation age and gestational diabetes mellitus. Other complications are contemplated.

In certain embodiments, the maternal donor is the subject.

In certain embodiments, the maternal donor is not the subject. In certain embodiments, the maternal donor is a donor of an oocyte to be implanted in the subject.

In certain embodiments, the pregnancy comprises the use of an assisted reproductive technology.

The term “assisted reproduction” refers to a technique involving the production of an embryo from an oocyte or other cell, such that the embryo is capable of implantation. For example, an assisted reproduction technology includes a technique using an oocyte in vitro, in vitro fertilization (IVF; aspiration of an oocyte, fertilization in the laboratory and transfer of the embryo into a recipient), gamete intrafallopian transfer (GIFT; placement of oocytes into the fallopian tube), zygote intrafallopian transfer (ZIFT; placement of fertilized oocytes into the fallopian tube), tubal embryo transfer (TET; the placement of cleaving embryos into the fallopian tube), peritoneal oocyte and sperm transfer (POST; the placement of oocytes and sperm into the pelvic cavity), intracytoplasmic sperm injection (ICSI), testicular sperm extraction (TESE), microsurgical epididymal sperm aspiration (MESA), nuclear transfer, expansion from a totipotent stem cell, and parthenogenic activation. Other types of assisted reproductive technologies are contemplated. Methods of assisted reproduction are known in the art.

In certain embodiments, the initial classifying comprises classifying the risk in the subject as a low risk or an increased risk based on initial information comprising (i) initial genetic information and/or (ii) initial clinical information and/or (iii) initial lifestyle information, the initial information being from the subject and/or the maternal donor and/or the paternal donor.

In certain embodiments, the further classifying comprises classifying the risk in the subject having said increased risk as a moderate risk or a high risk based on information comprising (i) further genetic information and/or (ii) further clinical information and/or (iii) further lifestyle information, the further information being from the subject and/or the maternal donor and/or the paternal donor.

In certain embodiments, the initial classifying comprises classifying the risk in the subject as a low risk or an increased risk based on initial information comprising (i) initial genetic information and/or (ii) initial clinical information and/or (iii) initial lifestyle information, the initial information being from the subject and/or the maternal donor and/or the paternal donor, and the further classifying comprises classifying the risk in the subject having said increased risk as a moderate risk or a high risk based on information comprising (i) further genetic information and/or (ii) further clinical information and/or (iii) further lifestyle information, the further information being from the subject and/or the maternal donor and/or the paternal donor.

In certain embodiments, the genetic information comprises one or more of allelic information, RNA information (such as the expression of microRNAs), DNA methylation information, histone modification information and epigenetic information. Other types of genetic information are contemplated.

In certain embodiments, the genetic information comprises information relating to the presence and/or absence of one or more polymorphisms. In certain embodiments, the genetic information comprises information relating to the presence and/or absence of one or more single nucleotide polymorphisms.

In certain embodiments, the lifestyle information and/or clinical information comprises one or more of family history of a pregnancy complication, family history of hypertension, history of a previous miscarriage, time to conceive, presence of vaginal information, age, body mass index, subject's birth weight, maternal donor's birthweight, hyperemesis, gravidity, dose of folate (typically administered as folic acid), cervical length (eg transvaginal cervical length at 20 weeks gestation, ethnicity, use of barrier contraception (eg use of barrier contraception with paternal donor), snoring, computer usage, arterial pressure, subject's head circumference, type of work, hours work in paid employment, rhesus factor of the subject, type or work, work status of the partner of the subject, history of previous terminations, history of diabetes, diastolic blood pressure, pulse, glucose levels, folate dose, occurrence of vaginal bleeding (eg occurrence of vaginal bleeding continuing for at least 5 days), duration of sex without contraception before pregnancy, maternal height, any sister with a low birth weight baby, number of Lietz treatments, donor sperm or donor egg used in the pregnancy, chorionic villus sampling, amniocentesis, pre-eclamptic toxemia, history of pregnancy induced hypertension, height, gluclose levels (eg random glucose (mmol/L) at 15 weeks), waist size, mean arterial pressure, paternal age, Haematocrit testing (eg subject booking Haematocrit (PCV)), subject's birthweight, fertility treatment to conceive current pregnancy, any previous terminations (eg any previous terminations at >10 weeks), hormonal treatment to assist conception of current pregnancy, time of last colposcopy before conception of current pregnancy, fertility treatment (eg fertility treatment for PCOS prior to/at conception), paternal subject with type 2 diabetes, paternal subject with diabetes type not specified, family history of diabetes type 2, subject has a history of PET, bleeding gums (eg bleeding gums when brushing teeth at 15 weeks), and proteinuria (eg proteinuria at 15 weeks, consumption of other drugs, consumption of marijuana, consumption of alcohol (eg units of alcohol per week in the 1st trimester), consumption of cigarettes, consumption of fruit, consumption of drugs, anxiety measures, time of schooling, physical activity, exercise (eg number of stairs climbed), educational status (eg years of schooling), number of episodes of waking during a night's sleep, snoring, and emotional support. Other types of lifestyle or clinical information are contemplated. It will be appreciated that the classification of the type of information as lifestyle or clinical information is at the discretion of the suitable person.

In certain embodiments, the clinical information comprises one or more of family history of a pregnancy complication, family history of hypertension, history of a previous miscarriage, time to conceive, presence of vaginal information, age, body mass index, subject's birth weight, maternal donor's birthweight, hyperemesis, gravidity, dose of folate (typically administered as folic acid), cervical length (eg transvaginal cervical length at 20 weeks gestation, ethnicity, use of barrier contraception (eg use of barrier contraception with paternal donor), snoring, computer usage, arterial pressure, subject's head circumference, type of work, hours work in paid employment, rhesus factor of the subject, type or work, work status of the partner of the subject, history of previous terminations, history of diabetes, diastolic blood pressure, pulse, glucose levels, folate dose, occurrence of vaginal bleeding (eg occurrence of vaginal bleeding continuing for at least 5 days), duration of sex without contraception before pregnancy, maternal height, any sister with a low birth weight baby, number of Lietz treatments, donor sperm or donor egg used in the pregnancy, chorionic villus sampling, amniocentesis, pre-eclamptic toxemia, history of pregnancy induced hypertension, height, glucose levels (eg random glucose (mmol/L) at 15 weeks), waist size, mean arterial pressure, paternal age, Haematocrit testing (eg subject booking Haematocrit (PCV)), subject's birthweight, fertility treatment to conceive current pregnancy, any previous terminations (eg any previous terminations at >10 weeks), hormonal treatment to assist conception of current pregnancy, time of last colposcopy before conception of current pregnancy, fertility treatment (eg fertility treatment for PCOS prior to/at conception), paternal subject with type 2 diabetes, paternal subject with diabetes type not specified, family history of diabetes type 2, subject has a history of PET, bleeding gums (eg bleeding gums when brushing teeth at 15 weeks), and proteinuria (eg proteinuria at 15 weeks).

In certain embodiments, the lifestyle information comprises one or more of consumption of marijuana, consumption of alcohol (eg units of alcohol per week in the 1st trimester), consumption of cigarettes, consumption of fruit, consumption of drugs, anxiety measures, time of schooling, physical activity, exercise (eg number of stairs climbed), educational status (eg years of schooling), number of episodes of waking during a night's sleep, snoring, and emotional support. Other types of lifestyle information are contemplated.

In certain embodiments, the lifestyle and/or clinical information comprises information at 20 or less weeks, 15 or less weeks, 14 or less weeks, 13 or less weeks, 12 or less weeks, 11 or less weeks or 10 or less weeks. In certain embodiments, the lifestyle and/or clinical information comprises information at 10 to 15 weeks, 11 to 15 weeks, 12 to 15 weeks, 13 to 15 weeks or 14 to 15 weeks.

In certain embodiments, the initial information and the further information is the same. In certain embodiments, the initial information and the further information is not the same.

In certain embodiments, the processing or classifying of the initial information and/or the processing or classifying of the further information comprises penalised logistic regression. Other statistical methods are contemplated.

In certain embodiments, the processing or classifying of the initial information and/or the further information comprises classifying the risk on the basis of a selected probability threshold. Other methods are contemplated.

In certain embodiments, the processing or classifying of the further information comprises classifying the risk on the basis of a selected probability threshold calculated from the initial information.

In certain embodiments, the methods comprise determining one or more of model coefficient estimates, estimated odds and corresponding 95% confidence intervals. Methods for determining coefficient estimates, estimated odds and corresponding 95% confidence intervals, are known in the art.

In certain embodiments, the complication of pregnancy comprises preeclampsia. The term “preeclampsia” refers to a condition with gestational hypertension (GHT) (blood pressure of 140/90 mm Hg or greater on at least 2 occasions 4 hours apart after 20 weeks' gestation) accompanied by proteinuria (300 mg/day or greater, or a spot protein creatinine ratio of 30 mg/mmol creatinine or greater).

In certain embodiments, the complication of pregnancy comprises preeclampsia and the initial clinical information and/or further clinical information comprises one or more of a family history of preeclampsia, a family history of chronic hypertension, a history of previous miscarriage, the time to conception, occurrence of vaginal bleeding (eg occurrence of vaginal bleeding continuing for at least 5 days), subject age, subject body mass index, mean arterial pressure, birth weight of the subject and/or the maternal donor, duration of sex without contraception before pregnancy, number of Lietz treatments, donor sperm or donor egg used in the pregnancy, chorionic villus sampling, amniocentesis, pre-eclamptic toxemia, history of pregnancy induced hypertension, and diastolic blood pressure.

In certain embodiments, the complication of pregnancy comprises preeclampsia and the initial lifestyle information and/or the further lifestyle information comprises one or more of alcohol consumption, cigarette consumption and consumption of fruit.

In certain embodiments, the complication of pregnancy comprises preeclampsia and the initial genetic information comprises no genetic information.

In certain embodiments, the complication of pregnancy comprises preeclampsia and the initial genetic information and/or further genetic information comprises genetic information from the maternal donor and the paternal donor.

In certain embodiments, the complication of pregnancy comprises preeclampsia and the initial genetic information and/or further genetic information comprises one or more of genetic information from one or more of the following genes: maternal AGT, maternal AGTR1, maternal IL10, paternal HIF1a, paternal MTRR, maternal MTHFR, maternal TGFB, maternal PGF, maternal PLG, maternal INSR, paternal NOS2A, paternal TP53, paternal MTHFR, paternal GSTP1, paternal INS, paternal TGFB, maternal PGF, paternal PGF, paternal CYP11A1, maternal INSR, and paternal MMP2.

In certain embodiments, the complication of pregnancy comprises preeclampsia and the initial genetic information and/or further genetic information comprises one or more of information from one or more of maternal AGT (rs 4762), maternal AGTR1 (rs5186), maternal IL10 (rs1800896), paternal HIF1a (rs11549465), maternal MTHFR (rs1801131), maternal PLG (rs2859879), maternal INSR (rs2059806), paternal NOS2A (rs1137933), paternal TP53 (rs1042522), paternal MTHFR (rs1800469), paternal INS (rs3842752), paternal TFGB (rs1800469), paternal PGF (rs1042886), maternal PGF (rs1042886), paternal MMP2 (rs243865), paternal GSTP1 (rs1695), paternal MTRR (rs1801394), maternal TGFB (rs1800469), paternal CYP11A1 (rs8039957).

In certain embodiments, the complication of pregnancy comprises preeclampsia and the initial lifestyle information comprises one or more of alcohol consumption (eg units of alcohol per week in the 1st trimester), cigarette consumption (eg number of cigarettes per day) and fruit consumption (eg frequency of consumption of fruit in the month prior to conception). In certain embodiments, the complication of pregnancy comprises preeclampsia and the initial lifestyle information comprises alcohol consumption (eg units of alcohol per week in the 1st trimester), cigarette consumption (eg number of cigarettes per day) and fruit consumption (eg frequency of consumption of fruit in the month prior to conception).

In certain embodiments, the complication of pregnancy comprises preeclampsia and the initial clinical information comprises one or more maternal age, mean arterial pressure, BMI, family history of PE, subject's birthweight, any previous miscarriage (eg any previous miscarriage at <=10 wks gestation with same man who has fathered the current pregnancy), and time to conceive (eg months to conceive). In certain embodiments, the complication of pregnancy comprises preeclampsia and the initial clinical information comprises maternal age, mean arterial pressure, BMI, family history of PE, subject's birthweight, any previous miscarriage (eg any previous miscarriage at <=10 wks gestation with same man who has fathered the current pregnancy), and time to conceive (eg months to conceive).

In certain embodiments, the complication of pregnancy comprises preeclampsia and the initial genetic information comprises no genetic information.

In certain embodiments, the complication of pregnancy comprises preeclampsia and the further lifestyle information comprises alcohol consumption (eg units of alcohol per week in the 1st trimester).

In certain embodiments, the complication of pregnancy comprises preeclampsia and the further clinical information comprises one or more of BMI, mean arterial pressure, any previous miscarriage (eg any previous miscarriage at <=10 wks gestation with same man who has fathered the current pregnancy), and family history of PE. In certain embodiments, the complication of pregnancy comprises preeclampsia and the further clinical information comprises BMI, mean arterial pressure, any previous miscarriage (eg any previous miscarriage at <=10 wks gestation with same man who has fathered the current pregnancy), and family history of PE.

In certain embodiments, the complication of pregnancy comprises preeclampsia and the further genetic information comprises maternal and paternal genetic information.

In certain embodiments, the complication of pregnancy comprises preeclampsia and the further genetic information comprises genetic information from one or more of maternal AGTR1, maternal IL10, paternal NOS2A, paternal TP53, maternal MTHFR, paternal GSTP1, maternal TGFB, and paternal CYP11A1. In certain embodiments, the complication of pregnancy comprises preeclampsia and the further genetic information comprises information from maternal AGTR1, maternal IL10, paternal NOS2A, paternal TP53, maternal MTHFR, paternal GSTP1, maternal TGFB, and paternal CYP11A1.

In certain embodiments, the complication of pregnancy comprises preeclampsia and the further genetic information comprises genetic information from one or more of maternal AGTR1 (rs5186), maternal IL10 (rs1800896), paternal NOS2A (rs1137933), paternal TP53 (rs1042522), maternal MTHFR (rs1801131), paternal GSTP1 (rs1695), maternal TGFB (rs1800469), and paternal CYP11A1 (rs8039957). In certain embodiments, the complication of pregnancy comprises preeclampsia and the further genetic information comprises genetic information from maternal AGTR1 (rs5186), maternal IL10 (rs1800896), paternal NOS2A (rs1137933), paternal TP53 (rs1042522), maternal MTHFR (rs1801131), paternal GSTP1 (rs1695), maternal TGFB (rs1800469), and paternal CYP11A1 (rs8039957).

In certain embodiments, the complication of pregnancy comprises preeclampsia and the further genetic information comprises genetic information from maternal one or more of AGTR1 (rs5186_CC), maternal IL10 (rs1800896_AA), paternal NOS2A (rs1137933_CC), paternal TP53 (rs1042522_GG), maternal MTHFR (rs1801131_CC), paternal GSTP1 (rs1695_GG), maternal TGFB (rs1800469_AA), and paternal CYP11A1 (rs8039957_AA). In certain embodiments, the complication of pregnancy comprises preeclampsia and the further genetic information comprises genetic information from maternal AGTR1 (rs5186_CC), maternal IL10 (rs1800896_AA), paternal NOS2A (rs1137933_CC), paternal TP53 (rs1042522_GG), maternal MTHFR (rs1801131_CC), paternal GSTP1 (rs1695_GG), maternal TGFB (rs1800469_AA), and paternal CYP11A1 (rs8039957_AA).

In certain embodiments, the complication of pregnancy comprises preterm birth. The term “preterm birth” refers to birth at less than 37 weeks of gestation that is spontaneous and not a result of medical or obstetric intervention.

In certain embodiments, the complication of pregnancy comprises preterm birth and the initial clinical information and/or further clinical information comprises one or more of a maternal height, family history of low birth weight baby, any sister with a low birth weight baby, a family history of spontaneous preterm birth, anxiety measures, hospital admission due to hyperemesis, subject or maternal donor body mass index, subject or maternal gravidity, months to conceive, folate use, number of Lietz treatments, donor sperm or donor egg used in the pregnancy, chorionic villus sampling, amniocentesis, pre-eclamptic toxemia, history of pregnancy induced hypertension, and transvaginal length (eg transvaginal length at 20 weeks gestation).

In certain embodiments, the complication of pregnancy comprises preterm birth and the initial lifestyle information and/or the further lifestyle comprises one or more of number of household members, exercise (eg number of time climbed stairs in the last month), marijuana consumption, consumption of fruit, consumption of recreation drugs, educational status (eg years of schooling), extreme exercise, type of work, activities at work, state-trait anxiety, feeling in pregnancy, and immigration history.

In certain embodiments, the complication of pregnancy comprises preterm birth and the initial genetic information and/or further genetic information comprises genetic information from the maternal donor.

In certain embodiments, the complication of pregnancy comprises preterm birth and the initial genetic information and/or further genetic information comprises genetic information from one or more of a maternal AGT, maternal BCL2, maternal TCN2, maternal IGF2R, maternal uPA, maternal MMP2, maternal TIMP3, maternal ADD1, maternal MBL2, maternal FLT1, maternal IL1B, maternal IGF1R, maternal MMP9, maternal CYP11A1.

In certain embodiments, the complication of pregnancy comprises preterm birth and the initial genetic information and/or the further genetic information comprises genetic information from one or more of maternal AGT (rs4762), maternal BCL2 (rs2279115), maternal TCN2 (rs1801198), maternal IGF2R (rs2274849), maternal uPA (rs2227564), maternal MMP2 (rs243865), maternal TIMP3 (rs5749511), maternal ADD1 (rs4961), maternal MBL2 (rs1800450), maternal FLT1 (FLT1C677T), maternal IL1B (rs16944), maternal IGF1R (rs11247361), maternal MMP9 (rs3918242), maternal CYPA11A1 (rs4887139), and maternal CYPA11A1 (rs8039957).

In certain embodiments, the complication of pregnancy comprises preterm birth and the initial lifestyle information comprises one or more of level of exercise (eg extreme exercise in pregnancy (undertook vigrous exercise at least once a day), number of times climbed stairs in the last month, educational history (eg years of schooling), and immigration history. In certain embodiments, the complication of pregnancy comprises preterm birth and the initial lifestyle information comprises level of exercise (eg extreme exercise in pregnancy (undertook vigrous exercise at least once a day), number of times climbed stairs in the last month, educational history (eg years of schooling), and immigration history.

In certain embodiments, the complication of pregnancy comprises preterm birth and the initial clinical information comprises one or more of folate dose (eg folate dose per day in 1st trimester), maternal height, gravidity, time to conceive (eg months to conceive), family history of a low birth weight baby, whether the subject's mother had a history of PET, and any hospital admissions due to hyperemesis. In certain embodiments, the complication of pregnancy comprises preterm birth and the initial clinical information comprises folate dose (eg folate dose per day in 1st trimester), maternal height, gravidity, time to conceive (eg months to conceive), family history of a LBW baby, whether the subject's mother had a history of PET, and any hospital admissions due to hyperemesis.

In certain embodiments, the complication of pregnancy comprises preterm birth and the initial genetic information comprises genetic information from one or more maternal markers. In certain embodiments, the complication of pregnancy comprises preterm birth and the initial genetic information comprises genetic information from one or more maternal markers and no paternal markers.

In certain embodiments, the complication of pregnancy comprises preterm birth and the initial genetic information comprises genetic information from one or more of maternal AGT, maternal BCL2, maternal TCN2, maternal IGF2R, maternal IL1B, maternal uPA, maternal CYP11A1, maternal IGF1R, maternal MMP2, maternal MMP9, and maternal TIMP3. In certain embodiments, the complication of pregnancy comprises preterm birth and the initial genetic information comprises genetic information from maternal AGT, maternal BCL2, maternal TCN2, maternal IGF2R, maternal IL1B, maternal uPA, maternal CYP11A1, maternal IGF1R, maternal MMP2, maternal MMP9, and maternal TIMP3.

In certain embodiments, the complication of pregnancy comprises preterm birth and the initial genetic information comprises genetic information from one or more of maternal AGT (rsS4762), maternal BCL2 (rs2279115), maternal TCN2 (rs1801198), maternal IGF2R (rs2274849), maternal IL1B (rs16944), maternal uPA (rs2227564), maternal CYP11A1 (rs8039957), maternal IGF1R (rs11247361), maternal MMP2 (rs243865), maternal MMP9 (rs3918242), and maternal TIMP3 (rs5749511). In certain embodiments, the complication of pregnancy comprises preterm birth and the initial genetic information comprises genetic information from maternal AGT (rsS4762), maternal BCL2 (rs2279115), maternal TCN2 (rs1801198), maternal IGF2R (rs2274849), maternal IL1B (rs16944), maternal uPA (rs2227564), maternal CYP11A1 (rs8039957), maternal IGF1R (rs11247361), maternal MMP2 (rs243865), maternal MMP9 (rs3918242), and maternal TIMP3 (rs5749511).

In certain embodiments, the complication of pregnancy comprises preterm birth and the initial genetic information comprises genetic information from one or more of maternal AGT (rsS4762_TT), maternal BCL2 (rs2279115_AA), maternal TCN2 (rs1801198_CC), maternal IGF2R (rs2274849_GG), maternal IL1B (rs16944_GG), maternal uPA (rs2227564_TT), maternal CYP11A1 (rs8039957_AA), maternal IGF1R (rs11247361_CC), maternal MMP2 (rs243865_CC), maternal MMP9 (rs3918242_CC), and maternal TIMP3 (rs5749511_CC). In certain embodiments, the complication of pregnancy comprises preterm birth and the initial genetic information comprises genetic information from maternal AGT (rsS4762_TT), maternal BCL2 (rs2279115_AA), maternal TCN2 (rs1801198_CC), maternal IGF2R (rs2274849_GG), maternal IL1B (rs16944_GG), maternal uPA (rs2227564_TT), maternal CYP11A1 (rs8039957_AA), maternal IGF1R (rs11247361_CC), maternal MMP2 (rs243865_CC), maternal MMP9 (rs3918242_CC), and maternal TIMP3 (rs5749511_CC).

In certain embodiments, the complication of pregnancy comprises preterm birth and the further lifestyle information comprises one or more of level of exercise (eg extreme exercise in pregnancy (vigrous exercise at least once a day), number of times climbed stairs in the last month), educational history (eg years of schooling), and immigration history. In certain embodiments, the complication of pregnancy comprises preterm birth and the initial lifestyle information comprises level of exercise (eg extreme exercise in pregnancy (undertook vigrous exercise at least once a day), number of times climbed stairs in the last month, educational status or history (eg years of schooling), and immigration history.

In certain embodiments, the complication of pregnancy comprises preterm birth and the further clinical information comprises one or more of folate dose (eg folate dose per day in 1st trimester), maternal height, gravidity, a family history of a low birth weight baby, and whether the subject's mother had a history of PET. In certain embodiments, the complication of pregnancy comprises preterm birth and the further clinical information comprises folate dose (eg folate dose per day in 1st trimester), maternal height, gravidity, a family history of a low birth weight baby, and whether the subject's mother had a history of PET.

In certain embodiments, the complication of pregnancy comprises preterm birth and the further genetic information comprises genetic information from one or more maternal markers. In certain embodiments, the complication of pregnancy comprises preterm birth and the further genetic information comprises genetic information from one or more maternal markers and no paternal markers.

In certain embodiments, the complication of pregnancy comprises preterm birth and the further genetic information comprises genetic information from one or more of maternal AGT, maternal BCL2, maternal TCN2, maternal IGF2R, maternal IL1B, maternal uPA, maternal CYP11A1, maternal IGF1R, maternal MMP2, maternal MMP9, and maternal TIMP3. In certain embodiments, the complication of pregnancy comprises preterm birth and the further genetic information comprises genetic information from maternal AGT, maternal BCL2, maternal TCN2, maternal IGF2R, maternal IL1B, maternal uPA, maternal CYP11A1, maternal IGF1R, maternal MMP2, maternal MMP9, and maternal TIMP3.

In certain embodiments, the complication of pregnancy comprises preterm birth and the further genetic information comprises genetic information from one or more of maternal AGT (rs4762), maternal BCL2 (rs2279115), maternal TCN2 (rs1801198), maternal IGF2R (rs2274849), maternal IL1B (rs16944), maternal uPA (rs2227564), maternal CYP11A1 (rs8039957), maternal IGF1R (rs11247361), maternal MMP2 (rs243865), maternal MMP9 (rs3918242), and maternal TIMP3 (rs5749511). In certain embodiments, the complication of pregnancy comprises preterm birth and the further genetic information comprises genetic information from maternal AGT (rs4762), maternal BCL2 (rs2279115), maternal TCN2 (rs1801198), maternal IGF2R (rs2274849), maternal IL1B (rs16944), maternal uPA (rs2227564), maternal CYP11A1 (rs8039957), maternal IGF1R (rs11247361), maternal MMP2 (rs243865), maternal MMP9 (rs3918242), and maternal TIMP3 (rs5749511).

In certain embodiments, the complication of pregnancy comprises preterm birth and the further genetic information comprises genetic information from one or more of maternal AGT (rs4762_TT), maternal BCL2 (rs2279115_AA), maternal TCN2 (rs1801198_CC), maternal IGF2R (rs2274849_GG), maternal IL1B (rs16944_GG), maternal uPA (rs2227564_TT), maternal CYP11A1 (rs8039957_AA), maternal IGF1R (rs11247361_CC), maternal MMP2 (rs243865_CC), maternal MMP9 (rs3918242_CC), and maternal TIMP3 (rs5749511_CC). In certain embodiments, the complication of pregnancy comprises preterm birth and the further genetic information comprises genetic information from maternal AGT (rs4762_TT), maternal BCL2 (rs2279115_AA), maternal TCN2 (rs1801198_CC), maternal IGF2R (rs2274849_GG), maternal IL1B (rs16944_GG), maternal uPA (rs2227564_TT), maternal CYP11A1 (rs8039957_AA), maternal IGF1R (rs11247361_CC), maternal MMP2 (rs243865_CC), maternal MMP9 (rs3918242_CC), and maternal TIMP3 (rs5749511_CC).

In certain embodiments, the complication of pregnancy comprises small for gestational age. The term “small for gestational age” refers to as a birthweight of less than the 10th customised centile, adjusted for maternal height, weight, parity, ethnicity, gestational age at delivery and infant sex.

In certain embodiments, the complication of pregnancy comprises small for gestational age and the initial clinical information and/or further clinical information comprises one or more of a family history of hypertension, ethnicity, subject and/or maternal donor body mass index, mean arterial pressure, diastolic blood pressure, subject and/or maternal donor's head circumference, and extent of vaginal bleeding.

In certain embodiments, the complication of pregnancy comprises small for gestation age and the initial lifestyle information and/or the further lifestyle comprises one or more of consumption of recreation drugs and/or alcohol, use of barrier conception, extent of snoring, fruit consumption, extent of computer usage, hours in employment, extent of smoking, smoking status, and the subject's rhesus factor.

In certain embodiments, the complication of pregnancy comprises small for gestational age and the initial genetic information and/or further genetic information comprises genetic information from the maternal and paternal donor.

In certain embodiments, the complication of pregnancy comprises small for gestational age and the initial genetic information and/or further genetic information comprises genetic information from one or more of maternal IL6, maternal F2, maternal NAT1, paternal NAT1, maternal INS, paternal TCN2, paternal THBS1, paternal IGF2, and paternal IGF2AS.

In certain embodiments, the complication of pregnancy comprises small for gestational age and the initial genetic information and/or further genetic information comprises genetic information from one or more of maternal IL6 (rs1800795), maternal F2 (rs1799963), maternal NAT1 (rs1057126), paternal NAT1 (rs1057126), maternal INS (rs3842752), paternal TCN2 (rs18001198), paternal THBS1 (rs2228262), paternal IGF2 (rs3741204) and paternal IGF2AS (rs1004446).

In certain embodiments, the complication of pregnancy comprises small for gestational age and the initial lifestyle information comprises one or more of cigarette consumption (eg total number of cigarettes a woman was exposed to in the 1st trimester), and fruit consumption (eg low fruit consumption in the month prior to conception). In certain embodiments, the complication of pregnancy comprises small for gestational age and the initial lifestyle information comprises cigarette consumption (eg total number of cigarettes a woman was exposed to in the 1st trimester), and fruit consumption (eg low fruit consumption in the month prior to conception).

In certain embodiments, the complication of pregnancy comprises small for gestational age and the initial clinical information comprises use of other drugs (eg used other recreational drugs or binge alcohol consumption (>=6 units/session)).

In certain embodiments, the complication of pregnancy comprises small for gestational age and the initial genetic information comprises no genetic information.

In certain embodiments, the complication of pregnancy comprises small for gestational age and the further lifestyle information comprises one or more of cigarette consumption (eg total number of cigarettes a woman was exposed to in the 1st trimester), and use of barrier contraception (eg use of barrier contraception with biological father of baby). In certain embodiments, the complication of pregnancy comprises small for gestational age and the further lifestyle information comprises cigarette consumption (eg total number of cigarettes a woman was exposed to in the 1st trimester), and use of barrier contraception (eg use of barrier contraception with biological father of baby).

In certain embodiments, the complication of pregnancy comprises small for gestational age and the further clinical information comprises one or more of use of other drugs (eg used other recreational drugs or binge alcohol consumption (>=6 units/session)), rhesus factor negative and maternal head circumference. In certain embodiments, the complication of pregnancy comprises small for gestational age and the further clinical information comprises use of other drugs (eg used other recreational drugs or binge alcohol consumption (>=6 units/session)), rhesus factor negative and maternal head circumference.

In certain embodiments, the complication of pregnancy comprises small for gestation age and the further genetic information comprises genetic information from one or more maternal markers and/or paternal markers.

In certain embodiments, the complication of pregnancy comprises small for gestational age and the further genetic information comprises genetic information from one or more of maternal IL6, maternal F2, maternal NAT1, paternal TCN2, maternal INS, and paternal IGF2AS. In certain embodiments, the complication of pregnancy comprises small for gestational age and the further genetic information comprises genetic information from maternal IL6, maternal F2, maternal NAT1, paternal TCN2, maternal INS, and paternal IGF2AS.

In certain embodiments, the complication of pregnancy comprises small for gestational age and the further genetic information comprises genetic information from one or more of maternal IL6 (rs1800795), maternal F2 (rs1799963), maternal NAT1 (rs1057126), paternal TCN2 (rs1801198), maternal INS (rs3842752), and paternal IGF2AS (rs1004446). In certain embodiments, the complication of pregnancy comprises small for gestational age and the further genetic information comprises genetic information from maternal IL6 (rs1800795), maternal F2 (rs1799963), maternal NAT1 (rs1057126), paternal TCN2 (rs1801198), maternal INS (rs3842752), and paternal IGF2AS (rs1004446).

In certain embodiments, the complication of pregnancy comprises small for gestational age and the further genetic information comprises genetic information from one or more of maternal IL6 (rs1800795_CC), maternal F2 (rs1799963_CC), maternal NAT1 (rs1057126_TT), paternal TCN2 (rs1801198_GG), maternal INS (rs3842752_CC), and paternal IGF2AS (rs1004446_TT). In certain embodiments, the complication of pregnancy comprises small for gestational age and the further genetic information comprises genetic information from maternal IL6 (rs1800795_CC), maternal F2 (rs1799963_CC), maternal NAT1 (rs1057126_TT), paternal TCN2 (rs1801198_GG), maternal INS (rs3842752_CC), and paternal IGF2AS (rs1004446_TT).

In certain embodiments, the complication of pregnancy comprises gestational diabetes mellitus. The term “gestational diabetes mellitus” refers to subjects with a fasting glucose of 5.1 mmol/L or higher in an Oral Glucose Tolerance Test, or a random glucose level of 11 nmol/L or higher.

In certain embodiments, the complication of pregnancy comprises gestational diabetes mellitus and the initial clinical information and/or further clinical information comprises one or more of previous terminations (eg previous terminations at >10 weeks), paternal donor's history of type 2 diabetes, subject or maternal body mass index, time to conception, diastolic blood pressure, pulse, glucose levels, folate dose, height, glucose level (eg random glucose (mmol/L) at 15 wks), waist size, mean arterial pressure, paternal age, Haematocrit testing (eg subject booking Haematocrit (PCV), subject's birthweight, fertility treatment to conceive current pregnancy, hormonal treatment to assist conception of current pregnancy, time of last colposcopy before conception of current pregnancy, fertility treatment for PCOS prior to/at conception, paternal subject with diabetes type not specified, family history of diabetes type 2, subject has a history of PET, and bleeding gums (eg bleeding gums when brushing teeth at 15 weeks, and proteinuria at 15 weeks).

In certain embodiments, the complication of pregnancy comprises gestational diabetes mellitus and the initial lifestyle information and/or the further lifestyle comprises one or more of consumption of fruit, education status (eg years of schooling), alcohol consumption (eg units of alcohol per week in the 1st trimester), exercise (eg number of times climbed stairs in the last month), number of episodes of waking during a night's sleep, snoring, and emotional support.

In certain embodiments, the complication of pregnancy comprises gestational diabetes mellitus and the initial genetic information and/or further genetic information comprises genetic information from the maternal donor.

In certain embodiments, the complication of pregnancy comprises gestational diabetes mellitus and the initial genetic information and/or further genetic information comprises genetic information maternal AGT, maternal FTO, maternal NOS2A, maternal PTEN, Maternal CYP24A1, maternal XRCC2, maternal ANGPT1, maternal KDR, maternal CYP11A, and maternal H19.

In certain embodiments, the complication of pregnancy comprises gestational diabetes mellitus and the initial genetic information and/or further genetic information comprises genetic information from one or more of maternal AFT (rs4762), maternal FTO (rs9939609), maternal NOS2A (rs1137933), maternal PTEN (rs2673832), maternal CYP24A1 (rs2248137), maternal XRCC2 (rs3218536), maternal ANGPT1 (rs2071559), maternal KDR (rs2071559), maternal CYP11A (rs8039957) and maternal H19 (rs2839701).

In certain embodiments, the complication of pregnancy comprises gestational diabetes mellitus and the initial lifestyle information comprises one or more of fruit consumption (eg >=3 times per day) fruit consumption in the month prior to conception; >=3 times per day) fruit consumption at 15 wks), and educational status (eg years schooling). In certain embodiments, the complication of pregnancy comprises gestational diabetes mellitus and the initial lifestyle information comprises fruit consumption (eg >=3 times per day) fruit consumption in the month prior to conception; >=3 times per day) fruit consumption at 15 wks), and educational status (eg years schooling).

In certain embodiments, the complication of pregnancy comprises gestational diabetes mellitus and the initial clinical information comprises one or more of folate dose (eg folate dose (μg per day) in 1st trimester); folate dose (μg per day) at 15 wks), diastolic blood pressure (eg diastolic blood pressure at first visit), maternal BMI, maternal height, pulse rate, glucose level (eg random glucose (mmol/L) measured by glucometer at 15 wks), mean arterial pressure, proteinuria (eg any proteinuria at 15 wks), exercise (eg number of times climbed stairs in the last month), snoring (eg snored most nights), emotional support, paternal age, haematocrit testing (eg booking Haematocrit (PCV)), maternal bodyweight, previous terminations (any previous terminations at >10 weeks), fertility treatment (eg fertility treatment to conceive current pregnancy), paternal diabetes (eg maternal father has type 2 diabetes; maternal father has diabetes type not specified), maternal family history of PET (eg participant's mother had a history of PET), and bleeding gums (eg bleeding gums when brushing teeth at 15 wks).

In certain embodiments, the complication of pregnancy comprises gestational diabetes mellitus and the initial clinical information comprises folate dose (eg folate dose (μg per day) in 1st trimester); folate dose (μg per day) at 15 wks), diastolic blood pressure (eg diastolic blood pressure at first visit), maternal BMI, maternal height, pulse rate, glucose level (eg random glucose (mmol/L) measured by glucometer at 15 wks), mean arterial pressure, proteinuria (eg any proteinuria at 15 wks), exercise (eg number of times climbed stairs in the last month), snoring (eg snored most nights), emotional support, paternal age, haematocrit testing (eg booking Haematocrit (PCV)), maternal bodyweight, previous terminations (any previous terminations at >10 weeks), fertility treatment (eg fertility treatment to conceive current pregnancy), paternal diabetes (eg maternal father has type 2 diabetes; maternal father has diabetes type not specified), maternal family history of PET (eg participant's mother had a history of PET), and bleeding gums (eg bleeding gums when brushing teeth at 15 wks).

In certain embodiments, the complication of pregnancy comprises gestational diabetes mellitus and the initial genetic information comprises no genetic information.

In certain embodiments, the complication of pregnancy comprises gestational diabetes mellitus and the further lifestyle information comprises one or more of fruit consumption (eg >=3 times per day) fruit consumption in the month prior to conception; >=3 times per day fruit consumption at 15 wks), and educational status (eg years schooling),

In certain embodiments, the complication of pregnancy comprises gestational diabetes mellitus and the further clinical information comprises one or more of folate dose (eg folate dose (μg per day) in 1st trimester; folate dose (μg per day) at 15 wks), diastolic blood pressure (eg diastolic blood pressure at first visit), maternal BMI, maternal height, pulse rate, glucose level (eg random glucose (mmol/L) measured by glucometer at 15 wks), time to conceive, previous terminations (any previous terminations at >10 weeks), maternal father diabetes (eg maternal father has type 2 diabetes, and maternal father has diabetes type not specified).

In certain embodiments, the complication of pregnancy comprises gestational diabetes mellitus and the further clinical information comprises folate dose (eg folate dose (μg per day) in 1st trimester; folate dose (μg per day) at 15 wks), diastolic blood pressure (eg diastolic blood pressure at first visit), maternal BMI, maternal height, pulse rate, glucose level (eg random glucose (mmol/L) measured by glucometer at 15 wks), time to conceive, previous terminations (any previous terminations at >10 weeks), and maternal father diabetes (eg maternal father has type 2 diabetes; maternal father has diabetes type not specified).

In certain embodiments, the complication of pregnancy comprises gestational diabetes mellitus and the further genetic information comprises genetic information from one or more maternal markers.

In certain embodiments, the complication of pregnancy comprises gestational diabetes mellitus and the further genetic information comprises genetic information from one or more of maternal AGT, maternal NOS2A, maternal CYP11A1, maternal CYP11A1, and maternal H19. In certain embodiments, the complication of pregnancy comprises gestational diabetes mellitus and the further genetic information comprises genetic information from maternal AGT, maternal NOS2A, maternal CYP11A1, maternal CYP11A1, and maternal H19. In certain embodiments, the complication of pregnancy comprises gestational diabetes and the further genetic information comprises genetic information from one or more of maternal AGT (rs4762), maternal NOS2A (rs1137933), maternal CYP11A1 (rs4887139), maternal CYP11A1 (rs8039957), and maternal H19 (rs2839701). In certain embodiments, the complication of pregnancy comprises gestational diabetes and the further genetic information comprises genetic information from maternal AGT (rs4762), maternal NOS2A (rs1137933), maternal CYP11A1 (rs4887139), maternal CYP11A1 (rs8039957), and maternal H19 (rs2839701).

In certain embodiments, the complication of pregnancy comprises gestational diabetes mellitus and the further genetic information comprises genetic information from one or more of maternal AGT (rs4762_TT), maternal NOS2A (rs1137933_TT), maternal CYP11A1 (rs4887139_AA), maternal CYP11A1 (rs8039957_GG), and maternal H19 (rs2839701_GG). In certain embodiments, the complication of pregnancy comprises gestational diabetes mellitus and the further genetic information comprises genetic information from maternal AGT (rs4762_TT), maternal NOS2A (rs1137933_TT), maternal CYP11A1 (rs4887139_AA), maternal CYP11A1 (rs8039957_GG), and maternal H19 (rs2839701_GG).

In certain embodiments, the pregnancy of complication comprises preeclampsia and the processing and/or classifying of the initial information comprises use of a model as described in Table 5 and/or the processing and/or classifying of the further information comprises use of a model as described in Table 6.

In certain embodiments, the pregnancy of complication comprises preterm birth and the processing and/or classifying of the initial information comprises use of a model as described in Table 7 and/or the processing and/or classifying of the further information comprises use of a model as described in Table 8.

In certain embodiments, the pregnancy of complication comprises small for gestational age and the processing and/or classifying of the initial information comprises use of a model as described in Table 9 and/or the processing and/or classifying of the further information comprises use of a model as described in Table 10.

In certain embodiments, the pregnancy of complication comprises gestational diabetes mellitus and the processing and/or classifying of the initial information comprises use of a model as described in Table 11 and/or the processing and/or classifying of the further information comprises use of a model as described in Table 12.

In certain embodiments, the methods comprise obtaining a sample from the maternal donor and/or the paternal donor and processing the sample to obtain genetic information. In certain embodiments, the methods comprise obtaining a sample from the maternal donor and/or the paternal donor and processing the sample to obtain the initial genetic information and/or further genetic information.

The term “gene” as used throughout the specification refers to a region of DNA, such as a genomic nucleotide sequence (nuclear or mitochondrial), and associated with a coding region and/or producing a transcript, and includes regulatory regions (e.g. promoter regions), transcribed regions, exons, introns, untranslated regions and other functional and/or non-functional sequence regions associated with the gene.

The term “gene” as used throughout the specification refers to a region of DNA, such as a genomic nucleotide sequence (nuclear or mitochondrial), and associated with a coding region and/or producing a transcript, and includes regulatory regions (e.g. promoter regions), transcribed regions, exons, introns, untranslated regions and other functional and/or non-functional sequence regions associated with the gene.

The term “polymorphism” refers to a difference in DNA sequence between individuals. Examples of types of polymorphisms include single nucleotide polymorphisms, a minisatellite length polymorphism, an insertion, a deletion, a frameshift, a base substitution, a duplication, an inversion, and a translocation.

The terms “amplification” or “amplify” (or variants thereof) refers to the production of additional copies of a nucleic acid sequence. For example, amplification may be achieved using polymerase chain reaction (PCR) technologies (as described in Dieffenbach, C. W. and G. S. Dveksler (1995) PCR Primer, a Laboratory Manual, Cold Spring Harbor Press, Plainview, N.Y.) or by other methods of amplification, such as rolling circle amplification on circular templates, such as described in Fire, A. and Xu, S-Q. (1995) Proc. Natl. Acad. Sci 92:4641-4645.

The term “nucleic acid” refers to a polynucleotide or oligonucleotide, being composed of deoxyribonucleotides and/or ribonucleotides in either single- or double-stranded form, including known analogues of natural nucleotides.

The term “rs” used in conjunction with an accession number refers to an entry in dbSNP database for genetic variation hosted by the National Center for Biotechnology Information (NCBI) in collaboration with the National Human Genome Research Institute (NHGRI). The database contains a range of molecular variations including (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants.

The term “hybridizes” or “hybridization” (or variants thereof) refers to a reaction in which one or more polynucleotides react to form a complex. Generally, the formation of such complexes involves stabilization via hydrogen bonding, for example between the bases of the nucleotide residues. In this regard, the hydrogen bonding may occur, for example, by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. Hybridisation may occur for example in solution, or between one nucleic acid sequence present in solution and another nucleic acid sequence immobilized on a solid support (e.g., membranes, filters, chips etc).

The term “stringent conditions” refers to the conditions that allow complementary nucleic acids to bind to each other within a range from at or near the Tm (Tm is the melting temperature) to about 20° C. below Tm. Factors such as the length of the complementary regions, type and composition of the nucleic acids (DNA, RNA, base composition), and the concentration of the salts and other components (e.g. the presence or absence of formamide, dextran sulfate and/or polyethylene glycol) must all be considered, essentially as described in Current Protocols in Molecular Biology, John Wiley & Sons, N.Y. (1989).

High stringent conditions are conditions under which a first and second oligonucleotide are allowed to hybridise such that the second oligonucleotide will bind specifically to a predetermined polymorphism sequence and not to a second, different, polynucleotide sequence. For example, the polymorphism to be detected may be an adenine nucleotide in a predetermined position of the polynucleotide molecule. Under high stringency conditions a probe containing a thymidine residue in the oligonucleotide will allow the oligonucleotide to hybridise to the polymorphism. Conversely, under the same high stringency conditions, an identical probe with the thymidine residue replaced with a guanosine residue, will not allow the oligonucleotide to hybridise to the polymorphism.

Low stringent conditions are conditions which allow a first and second oligonucleotide to hybridise despite some base mismatches occurring.

The present disclosure also includes within its scope veterinary applications. For example, the subject may be a mammal, a primate, a livestock animal (e.g. a horse, a cow, a sheep, a pig or a goat), a companion animal (e.g. a dog, a cat), a laboratory test animal (e.g. a mouse, a rat, a guinea pig, a rabbit), an animal of veterinary significance, or an animal of economic significance. Genetic information in other species equivalent to that in the human may be determined by a method known in the art.

The term “maternal donor” refers to a subject that provides an oocyte, or provides a cell that acts as a recipient for genetic material. In certain embodiments, the maternal donor is the same as the subject, and therefore the conception may have occurred naturally in the subject. In certain embodiments, the maternal donor is different to the subject, and the conceptions arises from assisted reproduction, such as in vitro fertilization or intracytoplasmic sperm injection (ICSI).

The term “paternal donor” as used throughout the specification is to be understood to mean a subject that provides a sperm cell, a progenitor of a sperm cell, or a nucleus for use in nuclear transfer.

Methods for determination of genetic information are known in the art. For example, methods are known in the art to determine allelic information, RNA information (such as the expression of microRNAs), DNA methylation information, histone modification information and epigenetic information. Other types of genetic information are contemplated.

For examples, methods of detection of polymorphisms include allele discrimination techniques, signal detection and assay formats, each of which are interchangeable and can be complementary.

Examples of allele discrimination techniques include allele specific hybridization, restriction digestion, enzymatic ligation, enzymatic polymerization and structure-specific cleavage. Examples of signal detection include radioactivity, mass detection, fluorescence detection, FRET, fluorescence polarization and chemiluminescence. Assay formats include sorting via charge, length or mass, sorting via arrays or sorting via optical spectra.

Other methods include for example RFLP, PCR, High Resolution Melt Curve Analysis (HRM), flap endonuclease, primer extension, 5′ nuclease, oligonucleotide ligase, single strand conformation polymorphism, temperature gradient capillary or gel electrophoresis, denaturing high performance liquid chromatography, sequencing or nucleic acid hybridisation. Methods for performing genetic testing including detection of polymorphisms are provided for example in Lorincz (2006) Nucleic Acid Testing for Human Disease. CRC Press, Boca Raton Fla., USA. Genetic information, such as polymorphisms, may be detected by mass spectrometry, for example matrix-assisted laser desorption/ionization time-of-flight (MALDI-TOF) mass spectrometry, for example as described in Tost and Gut (2005) Genotyping single nucleotide polymorphisms by MALDI mass spectrometry in clinical applications. Clinical Biochemistry 38(4): 335-350.

Specific genotyping platforms include for example TaqMan®, Pyrosequencing, DASH™, Invader®, MassEXTEND, Masscode™, SNPstream, SNPlex™, GoldenGate™, Padlock Probe and MW Assays, GeneChip®, Sequenom MassARRAY, luminex, DNA sequencing, RNA sequencing and whole gene genotyping.

If probes are used, they are usually polynucleotide fragments corresponding to the sequence surrounding the region of interest, such as a polymorphism. For example, an oligonucleotide complementary to different forms of a polymorphism may be used to differentiate between the polymorphic variants, if appropriate hybridization and washing conditions are chosen.

The stringency of conditions of hybridization can be established according to conventional protocols. Appropriate stringent conditions for each sequence may be established on the basis of well-known parameters such as temperature, composition of the nucleic acid molecules, salt conditions etc. For example see Sambrook et al., Molecular Cloning, A Laboratory Manual; ISBN: 0879695765, CSH Press, Cold Spring Harbor, 2001 and earlier edition Sambrook et al., Molecular Cloning, A Laboratory Manual; CSH Press, Cold Spring Harbor, 1989, or Higgins and Hames (eds.), Nucleic acid hybridization, a practical approach, IRL Press, Oxford 1985.

The probe may also be immobilized on a support. Fixation of the nucleic acid molecule to a solid support allows convenient handling of the test assay and for some solid supports such as chips, silica wafers or microtiter plates, allows for the simultaneous analysis of larger numbers of samples

A nucleic acid probe may also be linked to a detection agent, for example a radioactive, enzymatic, electrochemical, luminescent or fluorescent marker. Labelling of nucleic acids is well understood in the art and escribed, for example, in Sambrook et al., Molecular Cloning, A Laboratory Manual; ISBN: 0879695765, CSH Press, Cold Spring Harbor, 2001.

Multiple probes may also be used in the same hybridization reaction, wherein each probe is linked to a distinct detection agent so as to allow detection of multiple single nucleotide polymorphisms, detection of different polymorphisms at the same locus or detection of different polymorphisms on each allele.

In certain embodiments, the means for identifying genetic information, such as the presence of a polymorphism, includes using hybridization probes that are primers for PCR. For example, the primers may flank a polymorphism with the polymorphism determined by sequencing the PCR product. In certain embodiments, a sequence complementary to a polymorphism may be included in the primer, wherein hybridization to the template will only occur in the presence of the polymorphism in the template under high stringency conditions. Alternatively, a sequence complementary to a polymorphism may be included at the 3′ end of the primer, such that amplification of the template will only occur if a specific polymorphism is present.

Nucleic acid probes used as primers may also be linked to a detection agent, for example a radioactive, enzymatic, luminescent or fluorescent marker. If distinct probes are used to determine the alleles of the markers of the haplotype, then typically distinct detection agents, for example fluorophores each emitting at different wavelengths may be used.

Various other methods known in the art may be used to identify genetic information, such as a polymorphism. For example, DNA sequencing (either manual sequencing or automated fluorescent sequencing) or Next Generation Sequencing can be used to detect genetic information, such as a polymorphism. In this case, identification of the genetic information usually involves amplification of the region containing the genetic information from nucleic acid isolated from the subject (generally genomic DNA), although in some cases it may also possible to identify genetic information by sequencing a clone of the region derived from a particular subject, with or without amplification.

Another approach for identifying genetic information, such as polymorphisms, is the single-stranded conformation polymorphism assay (SSCA) (for example as described in Orita et al. (1989) Genomics 5(4): 874-879. This method does not detect all sequence changes, especially if the DNA fragment size is greater than 200 bp, but can be optimized to detect most DNA sequence variation. Fragments which have shifted mobility on SSCA gels are then sequenced to determine the exact nature of the DNA sequence variation.

Another approach is based on the detection of mismatches between two complementary DNA strands, including clamped denaturing gel electrophoresis (for example as described in Sheffield et al. (1991) Am. J. Hum. Genet. 49:699-706), heteroduplex analysis (for example as described in White et al. (1992) Genomics 12:301-303) and chemical mismatch cleavage (for example as described in Grompe et al. (1989) Proc. Natl. Acad. Sci. USA 86:5855-5892). Once a specific genetic variant is identified, an allele specific detection approach such as allele specific oligonucleotide hybridization can be utilized.

High resolution melt (HRM) is another technique that can be used to detect genetic information, such as polymorphisms. Methods of performing HRM are known in the art and include for example Herrman et al. (2006) Clinical Chemistry, 52(3):494-503.

If DNA sequence analysis is used to identify a genetic variant, such as a polymorphism, the presence of a variant in one allele (i.e. the subject is heterozygous for the polymorphism) will be identified by the presence of two different nucleotide sequences at the relevant position in the DNA sequence. Sequence of the DNA from a subject homozygous for an allele will yield only the presence of a nucleotide sequence at the relevant position of the DNA sequence.

To provide a suitable template for sequencing, a region of the genomic DNA isolated from the subject may be amplified, for example, using appropriately designed primers. Sequencing reactions with an appropriate primer and the analysis of the DNA sequence may be performed by a suitable method known in the art.

Alternatively, the presence of a genetic variant, such as a polymorphism, may be determined using sequence specific primers that will only amplify either the wild type allele or the allele with the variant from the DNA isolated from the subject. If sequence specific primers are used to amplify the DNA, a consensus primer and one of two alternative primers may be used. Each of the alternative primers will have a 3′ terminal nucleotide that either corresponds to the wild type sequence (a WT primer) or the polymorphic sequence (a SNP primer). In this case, amplification will only occur from the template having the correct complementary nucleotide.

Other methods to identify genetic information, such as a polymorphism, involve hybridization of nucleic acid containing the polymorphism with other nucleic acids (i.e. a reporter nucleic acid) that allows discrimination between differences in nucleic acid sequences. For example, Southern analysis with an oligonucleotide may be used to detect polymorphisms. Alternatively, methods are known in the art in which the oligonucleotide is attached to a solid substrate, such as chip, and the binding of a nucleic acid containing a polymorphism detected by binding (or lack thereof) to the oligonucleotide. In these cases, the identification of a polymorphism in a subject also includes detection of the polymorphism by hybridisation of nucleic acid derived to a reporter nucleic acid.

In certain embodiments, the identification of the presence of genetic variant, such as a polymorphism, in the maternal donor is by identifying the presence of the variant in one or more cells, such as a blood cell, a buccal cell, a cell from amniotic fluid, a cell from saliva, a germ cell (e.g. an oocyte), an ovarian follicular cell, and/or in cell-free nucleic acid, such as saliva. Cell-free DNA can also be used to identify genetic information, such as in a variety of biological fluids including blood, saliva, amniotic fluid, cervical fluid, semen. Methods of obtaining cells and screening for genetic information are known in the art and also as described herein.

Methods of obtaining maternal cells and screening for genetic information, such as polymorphisms, are as previously described herein. Methods of determining genetic information from a paternal donor are known in the art.

In one embodiment, identification of the presence of genetic information (such as a polymorphism) in the paternal donor is by identifying the presence of the genetic information in one or more of a blood cell, a buccal cell, a cell from semen, a cell from saliva, a germ cell and in cell-free nucleic acid, such as saliva.

Methods of obtaining paternal cells and screening for genetic information are as previously described herein.

In certain embodiments, the methods comprise obtaining a biological sample from the subject. The term “sample” refers to a sample obtained from a subject, or any derivative, extract, concentrate, mixture, or otherwise processed form thereof.

Methods for obtaining biological samples are known in the art. Examples of biological samples include biological fluids, blood samples, plasma samples, serum samples, urine samples, tear samples, saliva, swabs, buccal samples, hair samples, skin samples, dried blood, dried matrix, a biopsy, and fecal samples.

In certain embodiments, the biological sample is a biological fluid. In certain embodiments, the biological fluid comprises one or more of blood, plasma and serum. In certain embodiments, the biological fluid comprises one or more of maternal blood, maternal plasma and maternal serum.

In certain embodiments, the methods as described herein comprise using a computer processor means to determine the risk of a pregnancy complication occurring. Computer processor means are known in the art.

In certain embodiments, a computer processor means is used to process and/or classify the initial information and/or the further information.

In certain embodiments, the initial information and/or further information is received from at least one user device in data communication with the computer processor means over a network.

Certain embodiments of the present disclosure provide a method of preventing and/or treating a complication of pregnancy in a subject.

Certain embodiments of the present disclosure provide a method of preventing and/or treating a complication of pregnancy in a subject, the method comprising using a method as described herein to determine the risk of a complication of a pregnancy occurring and treating the subject on the basis of the risk so determined.

In certain embodiments, an antenatal intervention and/or a management strategy is used for a subject considered to be at risk for a complication of pregnancy.

Methods of treatment of pregnancy complications are described herein. In certain embodiments, antenatal care, management regimes and treatment options for some complications of pregnancy are as described in Example 8.

For example, a management strategy of increased monitoring and bed rest, and/or treatment with low dose aspirin may be used for a subject at moderate or high risk of suffering from preeclampsia or intrauterine growth restriction. For a subject considered to be at moderate or high risk of suffering from preterm birth, a management strategy of lifestyle changes, and/or treatment with vaginal progesterone may be used. For a subject considered to be at moderate or high risk of suffering from gestational diabetes, a management strategy of increased monitoring and lifestyle changes or be treated, and/or treatment with diabetes medicine or insulin may be used.

In certain embodiments, the complication of pregnancy comprises pre-eclampsia and the method comprises treating a subject at high risk with aspirin.

In certain embodiments, the complication of pregnancy comprises preterm birth and the method comprises treating a subject with moderate or high risk with progesterone when the subject comprises a cervical length of ≤25 mm.

In certain embodiments, the complication of pregnancy comprises gestational diabetes mellitus and the method comprises treating a subject at high risk with metformin.

Certain embodiments of the present disclosure provide a method of determining the risk of a complication of pregnancy occurring in a subject as described herein, using a computer processor means.

Certain embodiments of the present disclosure provide a method of determining the risk of a complication of pregnancy occurring in a subject, the pregnancy arising in the subject from a conception from a maternal donor and a paternal donor, the method comprising using a computer processor means to:
receive initial information comprising (i) initial genetic information and/or (ii) initial clinical information and/or (iii) initial lifestyle information, the initial information being from the subject and/or the maternal donor and/or the paternal donor, and process the initial information to classify the risk of a complication of pregnancy occurring in the subject as low risk or increased risk;
for the subject having said increased risk, receive further information, the further information comprising (i) further genetic information and/or (ii) further clinical information and/or (iii) further lifestyle information from the subject and/or the maternal donor and/or the paternal donor; and
process the further information to classify the risk in the subject having said increased risk as moderate risk or high risk; and
output the risk of the complication of pregnancy occurring in the subject.

Computer processing means are known in the art. Method for receiving data/information are known in the art.

In certain embodiments, the initial information and/or further information is received from at least one user device in data communication with the processor over a network. User devices are known in the art.

In certain embodiments, the method comprises transferring data associated with the initial and/or further information over the internet to a computer processing means.

In certain embodiments, the method comprises using a system for determining the risk of a complication of pregnancy as described herein.

Certain embodiments of the present disclosure provide a system for determining the risk of a complication of pregnancy as described herein, using a computer processor.

Systems utilising computer processors are known in the art. Examples are as described herein.

Certain embodiments of the present disclosure provide a system for determining the risk of a complication of pregnancy using a computer processor configured to process a method as described herein.

Certain embodiments of the present disclosure provide a system for determining the risk of a complication of pregnancy occurring in a subject, the pregnancy arising in the subject from a conception from a maternal donor and a paternal donor, the system comprising a computer processor configured to:
receive initial information comprising (i) initial genetic information and/or (ii) initial clinical information and/or (iii) initial lifestyle information, the initial information being from the subject and/or maternal donor and/or the paternal donor, and
process the initial information to classify the risk of a complication of pregnancy occurring in the subject as low risk or increased risk;
for the subject having said increased risk, the processor is further configured to receive further information, from the at least one user device or a further user device in data communication with the processor, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk; and
output the risk of the complication of pregnancy occurring in the subject.

In certain embodiments, the initial information is received from at least one user device in data communication with the processor over a network.

Certain embodiments of the present disclosure provide a system for determining the risk of a complication of pregnancy occurring in a subject, the pregnancy arising in the subject from a conception from a maternal donor and a paternal donor, the system comprising a computer processor configured to:
receive initial information from at least one user device in data communication with the processor over a network, the initial information comprising (i) initial genetic information and/or (ii) initial clinical information and/or (iii) initial lifestyle information, the initial information being from the subject and/or maternal donor and/or the paternal donor, and
process the initial information to classify the risk of a complication of pregnancy occurring in the subject as low risk or increased risk;
for the subject having said increased risk, the processor is further configured to receive further information, from the at least one user device or a further user device in data communication with the processor, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk; and
output the risk of the complication of pregnancy occurring in the subject.

Certain embodiments of the present disclosure provide a computer readable medium encoded with programming instructions executable by a computer processor means to process a method as described herein.

Certain embodiments of the present disclosure provide a computer-readable medium encoded with programming instructions executable by a computer processor means to allow the computer processor means to determine the risk of a complication of pregnancy occurring in a subject, as described herein.

Certain embodiments of the present disclosure provide a computer-readable medium system encoded with programming instructions executable by a computer processor means to allow the computer processor means to determine the risk of a complication of pregnancy occurring in a subject, the pregnancy arising in the subject from a conception from a maternal donor and a paternal donor, wherein the instructions allow the computer processing means to:
receive initial information comprising (i) initial genetic information and/or (ii) initial clinical information and/or (iii) initial lifestyle information, the initial information being from the subject and/or the maternal donor and/or the paternal donor, and
process the initial information to classify the risk of a complication of pregnancy occurring in the subject as low risk or increased risk; and
for the subject having said increased risk, receive further information, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk.

Computer-readable medium system encoded with programming instructions executable by a computer processor means are known in the art.

Certain embodiments of the present disclosure provide computer software encoded with programming instructions executable by a computer processor means to process a method as described herein.

Certain embodiments of the present disclosure provide computer software encoded with programming instructions executable by a computer processor means to allow the computer processor means to determine the risk of a complication of pregnancy occurring in a subject.

Certain embodiments of the present disclosure provide computer software encoded with programming instructions executable by a computer processor means to allow the computer processor means to determine the risk of a complication of pregnancy occurring in a subject, the pregnancy arising in the subject from a conception from a maternal donor and a paternal donor, wherein the software allows the computer processing means to:
receive initial information comprising (i) initial genetic information and/or (ii) initial clinical information and/or (iii) initial lifestyle information, the initial information being from the subject and/or the maternal donor and/or the paternal donor, and
process the initial information to classify the risk of a complication of pregnancy occurring in the subject as low risk or increased risk; and
for the subject having said increased risk, receive further information, the further information comprising (i) further genetic information and/or (ii) further clinical information and/or (iii) further lifestyle information, the further information being from the subject and/or the maternal donor and/or the paternal donor, and process the further information to classify the risk in the subject having said increased risk as moderate risk or high risk.

Standard techniques may be used for cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), herein incorporated by reference.

The present disclosure is further described by the following examples. It is to be understood that the following description is for the purpose of describing particular embodiments only and is not intended to be limiting with respect to the above description.

### Example 1—Patient Selection for Study

The Screening for Pregnancy Endpoints (SCOPE) study recruited nulliparous women with singleton pregnancies between November 2004 and September 2008 in Adelaide, Australia and Auckland, New Zealand. Ethical approval was obtained from local ethics committees (Australia REC 1712/5/2008, New Zealand AKX/02/00/364) and all women provided written informed consent. SCOPE aimed to develop tests to predict risk for preeclampsia, preterm birth and small for gestational age (for example as described in Kho, E. M., McCowan, L. M. E., North, R. A., Roberts, C. T., Chan, E., Black, M. A., Taylor, R. S. and Dekker, G. A., 2009. Duration of sexual relationship and its effect on preeclampsia and small for gestational age perinatal outcome, Journal of Reproductive Immunology. 82, 66-73).

Women were invited to participate prior to 15 weeks' gestation when attending hospital antenatal clinics, obstetricians, general practitioners or community midwives, and were interviewed and examined by a research midwife at 15±1 and 20±1 weeks of gestation.

The exclusion criteria included women who were considered to be at high risk of pre-eclampsia, small for gestational age (SGA) or preterm birth (PTB) due to underlying medical conditions (e.g. chronic hypertension requiring antihypertensive medication or diabetes), previous cervical knife cone biopsy, three terminations or three miscarriages or their pregnancy was complicated by a known major fetal anomaly or abnormal karyotype, or if they received interventions that may modify pregnancy outcome (e.g. aspirin, cervical suture).

Details of maternal age, BMI and socioeconomic index (SEI), medical and family history, along with dietary and lifestyle questionnaires with self-reported marijuana and cigarette smoking were recorded at 15 weeks' and 20 weeks' gestation and entered into an internet-accessed, password-protected centralised database with a complete audit trail (MedSciNetAB, Stockholm, Sweden).

Marijuana and cigarette smoking status were classified into five categories (i.e. never, quit prior to pregnancy, quit prior to 15 weeks' gestation, still using at 15 weeks' gestation, and still using at 20 weeks' gestation), with ‘non-smoking’ or ‘never used marijuana’ as the reference categories.

Spontaneous preterm birth (SPTB) was defined as birth at less than 37 weeks of gestation that was not a result of medical or obstetric intervention. Small for gestational age (SGA) was defined as a birthweight of less than the 10th customised centile, adjusted for maternal height, weight, parity, ethnicity, gestational age at delivery and infant sex. IUGR was defined as SGA <5th centile. Preeclampsia (PE) was defined as gestational hypertension (GHT) (blood pressure of 140/90 or greater on at least 2 occasions 4 hours apart after 20 weeks' gestation) accompanied by proteinuria (300 mg/day or greater, or a spot protein creatinine ratio of 30 mg/mmol creatinine or greater). Patients with a fasting glucose of 5.5 mmol/L or higher in a Glucose Tolerance Test, or a random glucose level of 11 nmol/L or higher, were defined as gestational diabetes mellitus (GDM).

Sample collection and genotyping were performed as described on Zhou et al (2013) Hum Reprod. 19(9): 618-627. Briefly, whole blood was collected into EDTA tubes from women at 15±1 weeks' gestation, from partners at some time during the woman's pregnancy and umbilical cord after delivery. Plasma and buffy coat were separated via centrifugation within 3 h of collection. Buccal swabs or saliva samples were collected from partners, who were unwilling to undergo venepuncture. The buccal swabs were applied to Whatman FTA cards (Whatman, USA) immediately following sample collection and saliva was collected using Oragene kits (DNA Genotek, USA).

Maternal and paternal blood was extracted from buffy coats isolated from peripheral or cord blood (QiAamp 96 DNA blood kit), Whatman FTA cards or from saliva (Oragene® DNA kits) following the manufacturers' instructions. Genotyping was conducted by the Australian Genome Research Facility using the Sequenom MassARRAY system. Quality controls were typically performed to ensure the accuracy of the genotyping data: (i) each sample was genotyped for Amelogenin, a sex-determinant gene and (ii) parental and neonatal genotyping data were checked for a Mendelian pattern of inheritance. Samples were excluded if an inconsistency between the sex of the sample and the corresponding Amelogenin genotype and/or non-Mendelian pattern of inheritance was observed. In addition, some samples were excluded due to inadequate blood samples, low quality of DNA or failure to genotype.

### Example 2—Prediction of Risk

Methods have been developed using a two-tiered approach that provides three tiers of risk: low, moderate and high risk.

The methods are based on four two-tiered algorithms which have been developed to predict risk for PE, PTB, IUGR and GDM with excellent prognostic capacity, sensitivity, specificity and positive and negative predictive values (PPV and NPV)]. Women are deemed to be at low risk, moderate risk or high risk.

Tier 1 of the algorithms distinguishes women at risk or at low risk and has maximum sensitivity (typically 87-92%). Tier 2 distinguishes women at moderate or high risk with maximum specificity (typically 91-95%). Together the PPV is typically above 20% (20-24%), a level above that which leading international clinicians would consider sufficient to act, for example to prescribe low-dose aspirin to prevent early onset PE or vaginal progesterone therapy to prevent PTB. A single blood or saliva or buccal sample from each parent early in pregnancy may be used to ascertain genotypes to be utilised in the algorithms together with clinical and lifestyle data obtained at a patient interview.

For PE, IUGR and GDM these may be completed by, for example, 12-15 weeks gestation and enable commencement of low-dose aspirin treatment before 16 weeks gestation which has previously been shown to be effective in reducing the incidence of early onset PE.

PTB prediction requires the addition of trans-vaginal ultrasound measurement of cervical length at 18-20 weeks. Vaginal progesterone therapy to prevent PTB has been shown to reduce the rate of PTB in high risk women treated (those with a previous PTB) by ˜50%, but treatment needs to be commenced by 20 weeks.

The methods utilise a combination of one or more of clinical information, lifestyle information and genetic information.

In the current study, genetic information including single nucleotide polymorphisms (SNPs), a specific type of gene variant, was obtained and then combined with one or more of clinical, family history, socioeconomic and lifestyle factors.

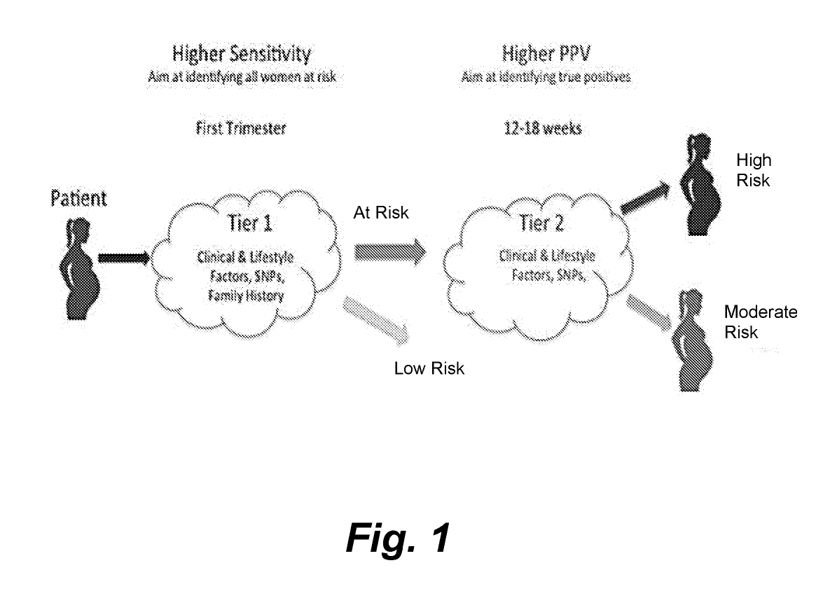

For each algorithm, two models were developed with penalized logistic regression based on predictors available at 15 weeks of gestation and included clinical, lifestyle and SNP predictors in Tier 1, and adding other predictors including cervical length at 20 weeks (specifically for the PTB algorithm) in Tier 2. Post-test probabilities were then calculated based on the Likelihood of each model using Bayes' theorem, and the final risk was classified into 3 levels (FIG. 1). For PE, IUGR and GDM prediction, all clinical and lifestyle data can be obtained at 12 weeks gestation, and the SNP data can be received within a week of sampling. Prediction of PTB additionally requires cervical length at 18-20 weeks gestation. This is important when considering appropriate interventions in women deemed at risk. SNPs contributed over 51% to PTB risk prediction, 44% for PE, 41% for IUGR<5th centile and about 35% for GDM risk prediction (as shown in Tables 1-4).

The SNPs selected included those in genes known to be involved in placental development, cancer, cardiovascular disease, one carbon (folate) metabolism, DNA synthesis and repair and thrombophilias among others. For PE and IUGR, both maternal and paternal SNPs were found to be required for risk prediction, reflecting a role for the father mediated by the placenta. Risk prediction for PTB and GDM included maternal, but not paternal SNPs.

TABLE 1
Performance of Prediction Algorithm for PTB

| | | Predicted | | |
|---|---|---|---|---|
| Tier 1 | Tier 2 | PTB | PTB case | % PTB |
| + | + | 25 | 6 | 24.00 |
| + | − | 284 | 13 | 4.58 |
| − | | 194 | 5 | 2.58 |

TABLE 2
Performance of Prediction Algorithm for PE

| | | Predicted | | |
|---|---|---|---|---|
| Tier 1 | Tier 2 | PE | PE case | % PE |
| + | + | 37 | 10 | 27.03 |
| + | − | 164 | 11 | 6.71 |
| − | | 86 | 1 | 1.16 |

TABLE 3
Performance of Prediction Algorithm for IUGR

| | | Predicted | | |
|---|---|---|---|---|
| Tier 1 | Tier 2 | IUGR | IUGR case | % IUGR |
| + | + | 21 | 7 | 33.33 |
| + | − | 262 | 13 | 4.96 |
| − | | 141 | 5 | 3.55 |

TABLE 4
Performance of Prediction Algorithm for GDM

| | | Predicted | | |
|---|---|---|---|---|
| Tier 1 | Tier 2 | GDM | GDM case | % GDM |
| + | + | 16 | 6 | 37.50 |
| + | − | 148 | 2 | 1.35 |
| − | | 159 | 2 | 1.26 |

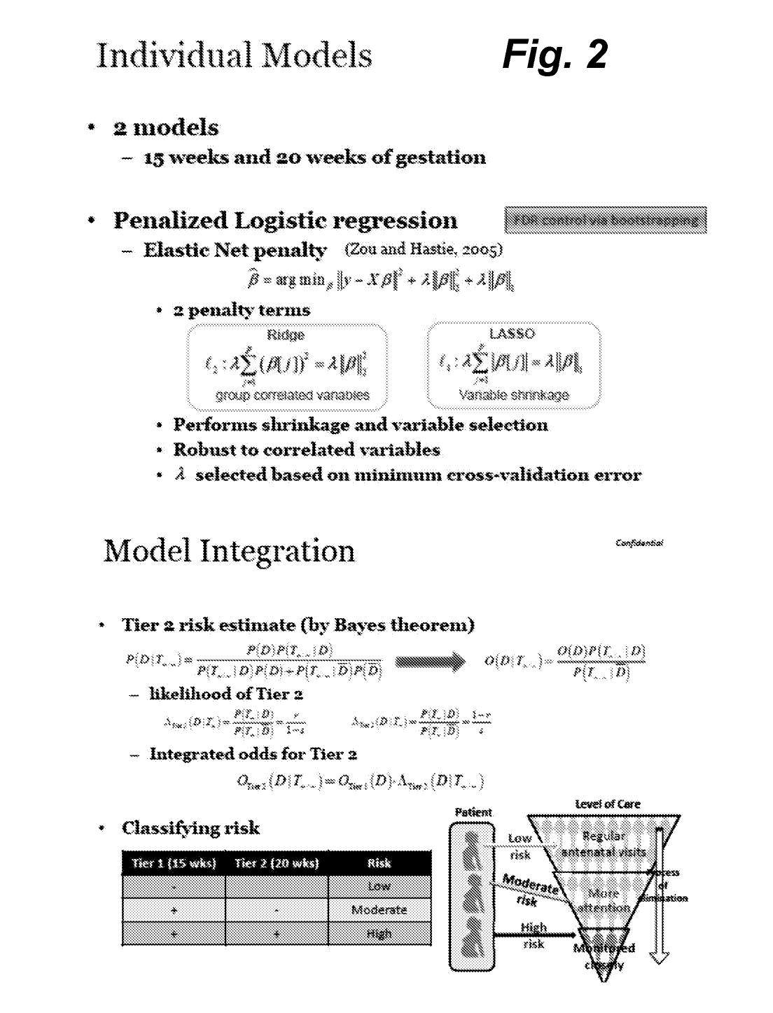

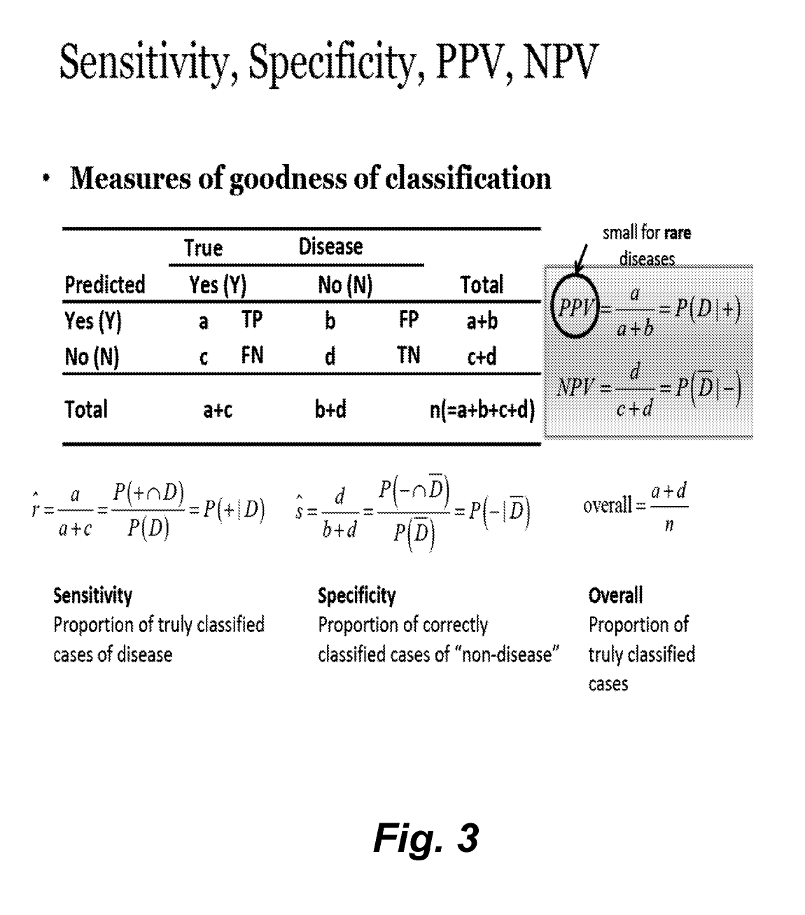

The determination of risk is based on a suite of algorithms with accompanying software to predict early in pregnancy the subsequent risk of the four main late pregnancy complications: preeclampsia, preterm birth, intrauterine growth restriction (IUGR) and gestational diabetes mellitus. The algorithms are based on genetic information (eg a number of single nucleotide polymorphisms (SNPs)) in both mother and father and clinical and lifestyle variables. Each prediction model takes a two-tiered approach with two independent algorithms that are integrated to provide three tiers of risk: low, moderate and high risk. Details of the individual models and integration are shown in FIG. 2. Details of sensitivity, specificity, positive predictive value and negative predictive value are shown in FIG. 3.

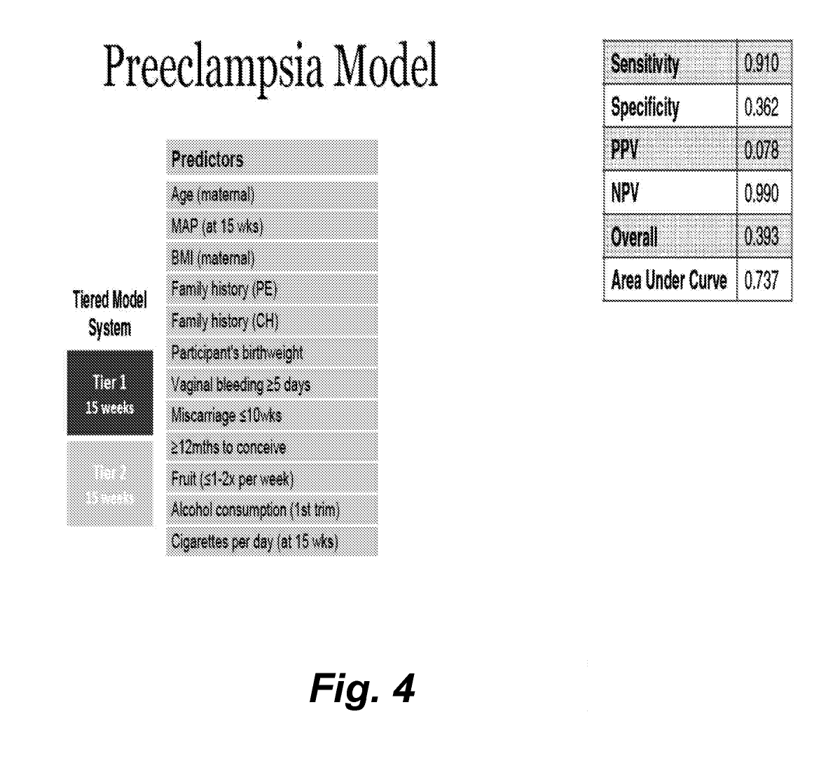

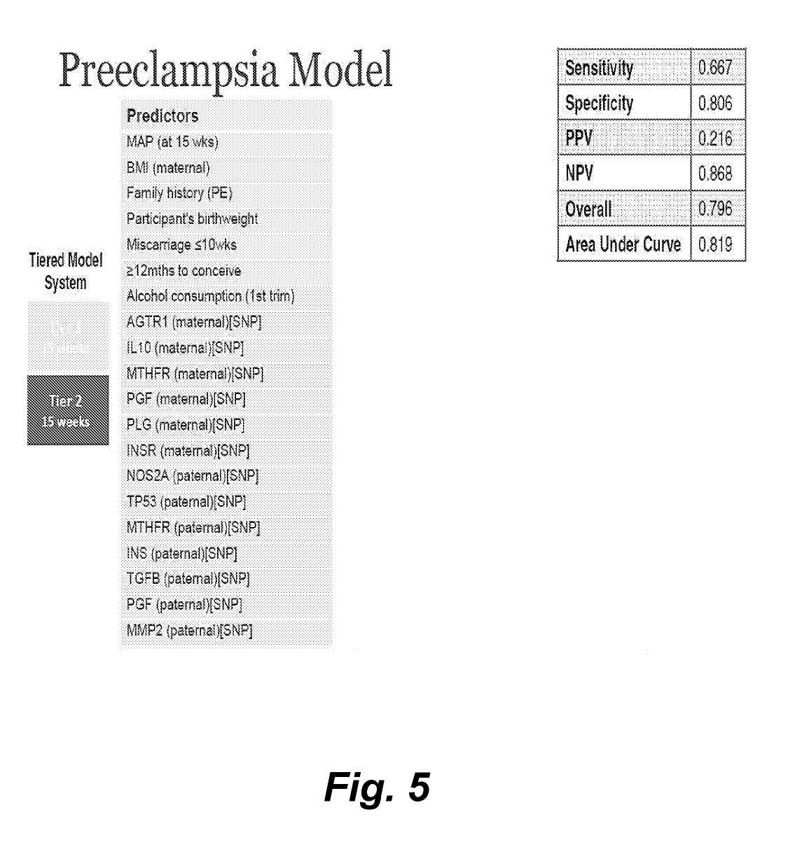

For example, for preeclampsia, tier 1 is as shown in FIG. 4, and tier 2 is as shown in FIG. 5

Web-based statistical software can be used to assesses each couple's parameters and provide a probability of risk of one or more of the four target pregnancy complications.

Tier 1 in each algorithm is directed to high sensitivity with a unique suite of variables for each disease. Tier 2 is applied only to women deemed at risk (versus low risk) in Tier 1 and is aimed at high positive predictive value (PPV). In certain embodiments, Tier 2 can include a subset of Tier 1 variables plus additional variables. Tiers 1 and 2 equations are then integrated, for example by Bayes Theorem, to identify women at moderate or high risk. Whilst the underlying principle is the same, equations and variables are different for each of the four pregnancy complications.

In clinical practice pregnant women and their partners provide a DNA sample (from blood, saliva or other biological sample) and answer a targeted questionnaire and undergo clinical measurements, typically at 10-15 weeks of pregnancy. DNA from both parents is genotyped for a suite of SNPs. The protocol may utilise a designated kit for SNP genotyping. In one embodiment, data is entered into web-based software and probability of disease is the output. The results of applying the algorithms also allows tailored antenatal care and preventative therapies in patients at high risk.

Whilst the genetic data described in the embodiments relates to SNPs, other types of genetic information, biomarkers such as proteins, DNA methylation can also be added.

It is to be noted that in some embodiments, for example preeclampsia and IUGR risk prediction, the algorithms may require access to paternal DNA for SNP genotyping.

The algorithms have been written in R and Java, but other software may be utilised, such as JavaScript or PHP for web-based application. In some embodiments, the software system includes a hosted/server based component. SQL has been used.

### Example 3—Methodology Overview

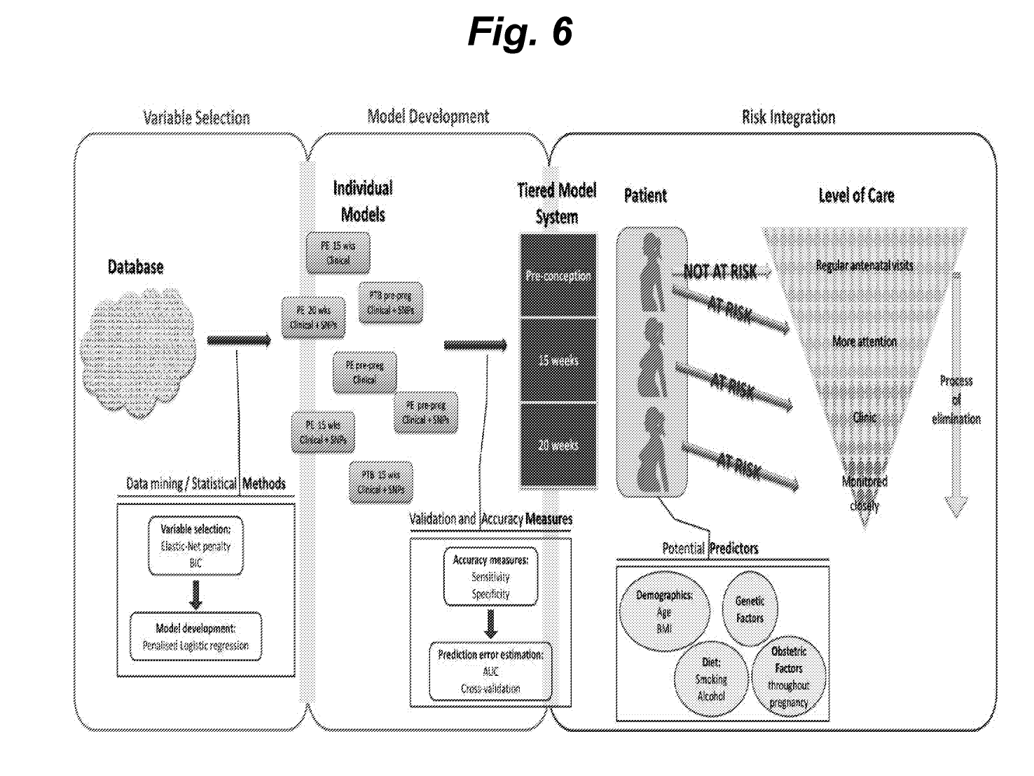

The development of prediction algorithms includes three stages: variable selection, model development, and risk integration (FIG. 6). All models for PE, SPTB, SGA, and GDM were developed based on the same methodology, but with different combinations of predictors, specific to the outcome of interest.

Since there are a large number of variables recorded in the SCOPE database, variable selection techniques have been applied to reduce the number of variables for development of practically sufficient prediction models. This includes Elastic-Net penalty and Akaike Information Criterion (AIC). Individual models with various combinations of clinical and SNP predictors, obtained up to 20 weeks of gestation, were then established based on variables selected. Each individual model was trained with model performance requirements specific to each tier, based on accuracy measures, such as sensitivity and specificity, and predictive values.

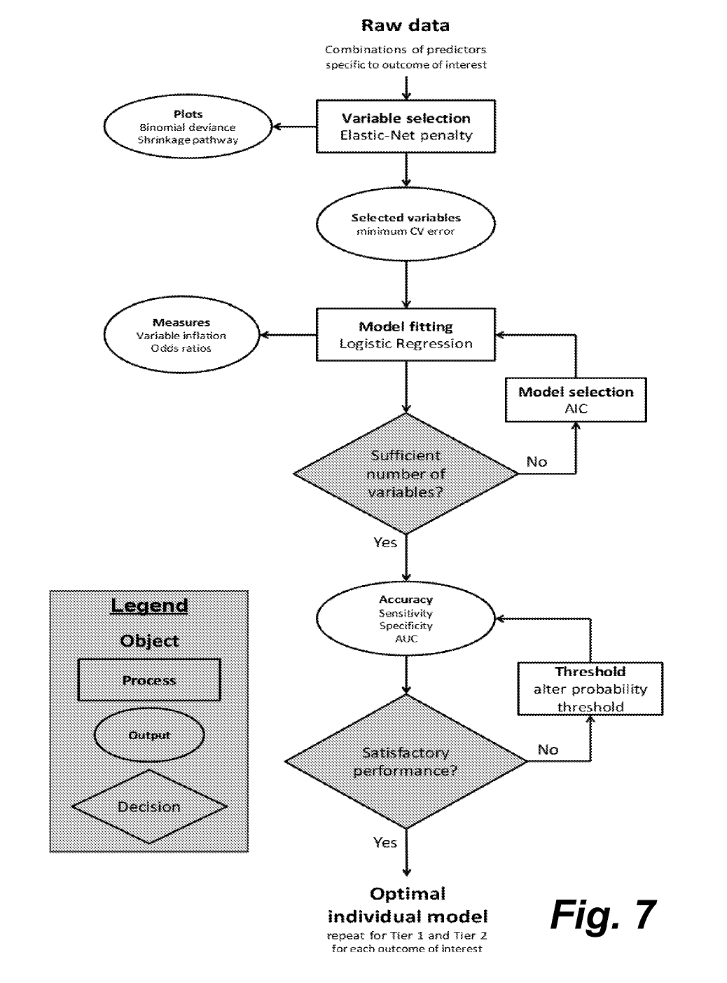

The best models were then integrated into a tiered prediction system (FIG. 7), which monitors and updates the predicted risk for individuals throughout pregnancy, when new predictors may be available or when changes occur. This is achieved by obtaining the posterior probability of Tier 2, based on the probability of Tier 1 as the prior probability, as well as the likelihood ratios calculated from the sensitivity and specificity of Tier 2. The probabilities were then further classified into 3 risk groups: low, moderate, and high risk.

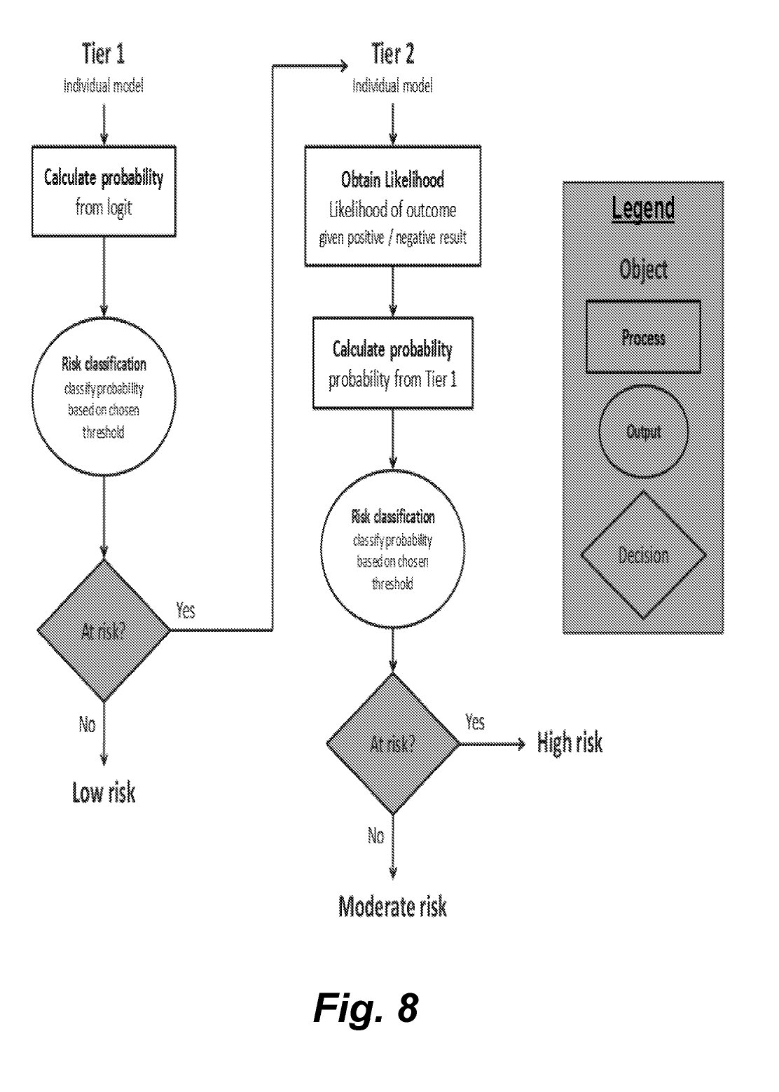

To establish an optimal individual model (FIG. 8), an initial set of variables which contain combinations of clinical measurements and SNPs, and in certain models, with variables specific to the outcome of interest (e.g. cervical length measurements for PTB). These variables went go through a variable selection procedure using Elastic-Net regularization, in which the deviance and cross-validation error were assessed for different combinations of variables along the variable shrinkage pathway.

The selected set of variables were fitted using Logistic regression, in which measures including odds ratios and variable inflations were assessed. In cases where the number of variables in the logistic regression model was still too large to be practically sufficient, a further model selection technique using Akaike Information Criterion (AIC) was performed.

Model performance was then assessed using accuracy measures including sensitivity, specificity and AUC. In most cases, the probability threshold needed to be altered to achieve the desired sensitivity and true positive rate specific to each tier.

After individual models for Tier 1 and Tier 2 were established, their predicted probabilities were then integrated (FIG. 8) to classify patients into low, moderate, or high risk. The predicted probability of Tier 1 was used to classify patients at low risk, i.e. patients who have a lower predicted probability then the chosen threshold. Patients who had a higher predicted probability proceed to Tier 2 prediction.

The positive and negative likelihood ratios in Tier 2 were first obtained, and multiplied by the predicted probability in Tier 1 as the prior probability. The resultant post-test probability was used to classify patients into moderate or high risk. Patients with a post-test probability lower than the chosen Tier 2 threshold were classified as moderate risk, while those with a probability higher than the threshold were classified as high risk.

Variable Selection

For all models, a backward-stepwise approach was used, in which predictors were eliminated from the full model (i.e. model with all predictors included) in each step. The selection process was controlled by penalty functions or regularization statistics, which aimed at eliminating variables that were considered unrelated or ineffective in improving the fit or prediction accuracy of models to prevent model over-fitting.

Variable Shrinkage

Elastic-net regularization has been used as a regularization method for variable shrinkage. This approach aimed at shrinking the coefficients of each predictor to 0 (i.e. variable-based). The coefficient estimates are given by \hat{β}=arg min_{β}∥y−Xβ∥²+λ₂∥β∥²+λ₁∥β∥₁, where λ is the tuning parameter, selected based on minimum cross-validation error for optimum prediction performance. It is worth noting that Elastic-Net is a 2-step penalty, involving both Ridge (l₂: λΣⱼ₌₁^{p}(β[j])²=λ₂∥β∥²) and Least Absolute Shrinkage and Selection Operator (LASSO) regression (l₁: λΣⱼ₌₁^{p}|β[j]|=λ₁∥β∥₁). Hence, both variable selection and shrinkage were performed, and it is robust to correlated variables. Since the purpose is to develop prediction models, the optimal sets of variables were chosen based on minimum cross-validation error.

A further model selection process using Akaike Information Criterion (AIC) was also performed in models where the number of variables was still considered large. This is a relative measure based on the likelihood function and the number of predictors in each step. It is given by AIC=2k−2 ln L(β), where k is the number of predictors in the current step and L(β)=Σᵢ₌₁ⁿ[yᵢβ^{T}xᵢ−log(1+e^{β}^{^{T}}^{x}^{ᵢ})] is the likelihood function. The optimal model with minimum AIC have an optimal number of predictors while maintaining a reasonable fit that describes the uncertainty.

Program Code for Variable Selection

The glmnet package in R (Friedman J, Hastie T and Tibshirani R (2010). Regularization Paths for Generalized Linear Models via Coordinate Descent. Journal of Statistical Software, 33(1), pp 1-22) was used to obtain the regularization paths for each variable in the models. The initial data were stored as a matrix in data.mat, and the coefficients were estimated for various λ in fit.1. The cross-validation errors were calculated in fit.1.cv, with the λ with minimum cross-validation error are then stored in lambda.min. This is then used to identify the optimal set of coefficients (Active.Coefficients.fit1).

Plots of binomial deviance, to assess error rates, and coefficient shrinkage pathways for various λ were also created.

[R] Code Snippet 2.2.1: glmnet

data.mat <− na.omit(data.matrix(data))
fit.1 <− glmnet(data.mat[,1:ncol(data.mat)−1],

data.mat[,ncol(data.mat)],family=“binomial”,alpha=0.5)

fit.1.cv <− cv.glmnet(data.mat,1:ncol(data.mat)−1],

data.mat[,ncol(x1)],

family=“binomial”,alpha=0.5)

Coefficients.fit1 <− coef(fit.1, s=fit.1.cv$lambda.min)
Active.Index.fit1 <− which(Coefficients.fit1 !=0)
Active.Coefficients.fit1 <−

rownames(Coefficients.fit1)[Active.Index.fit1]

plot(fit.1.cv)
plot(fit.1,xvar=“lambda”,label=T)
abline(v=log(fit.1.cv$lambda.min),col=“red”,lty=2)

Variable Shrinkage Pathways

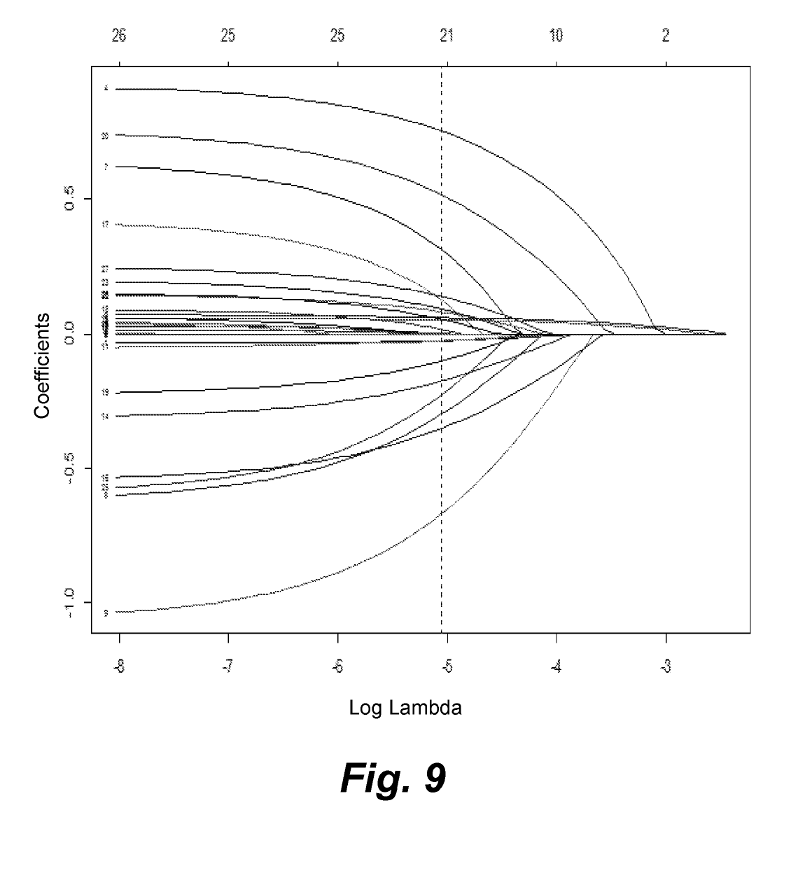

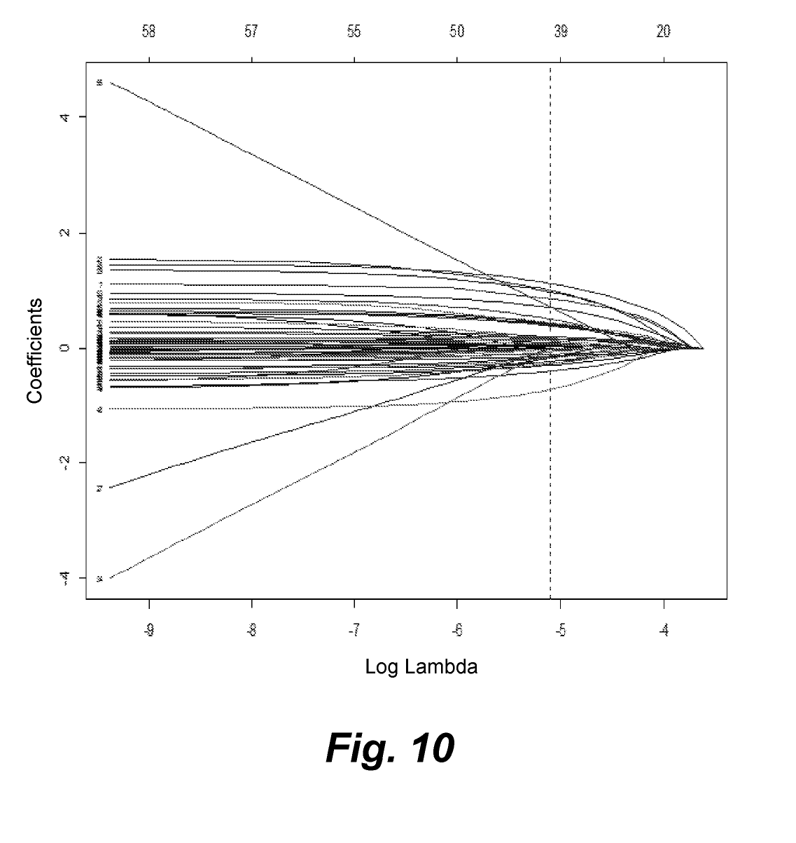

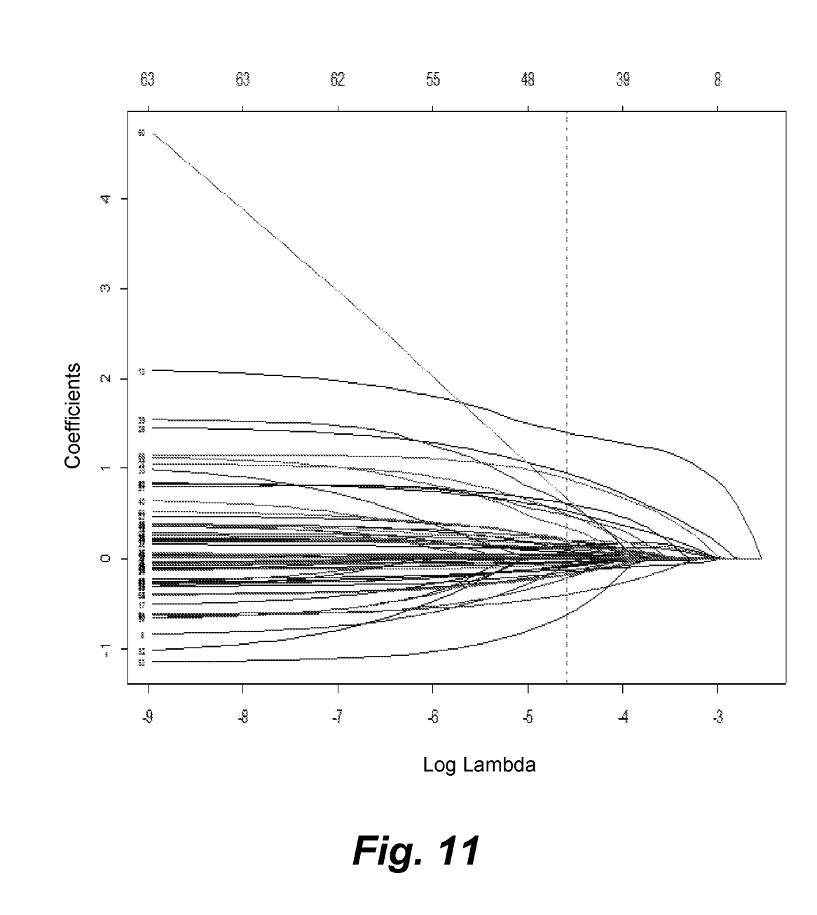

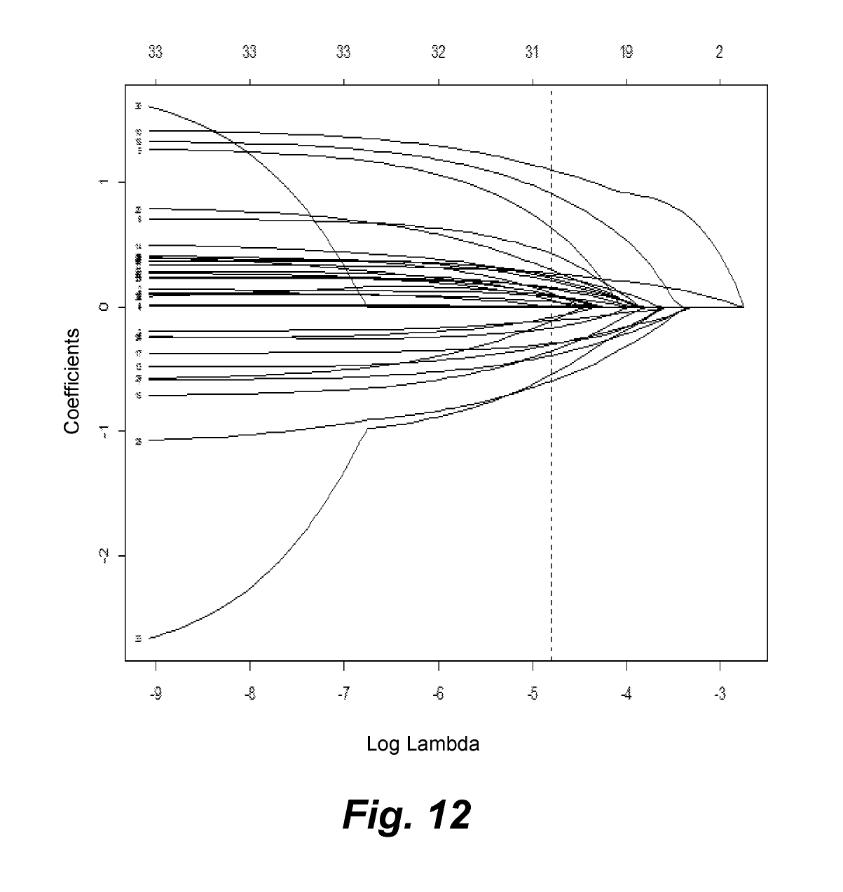

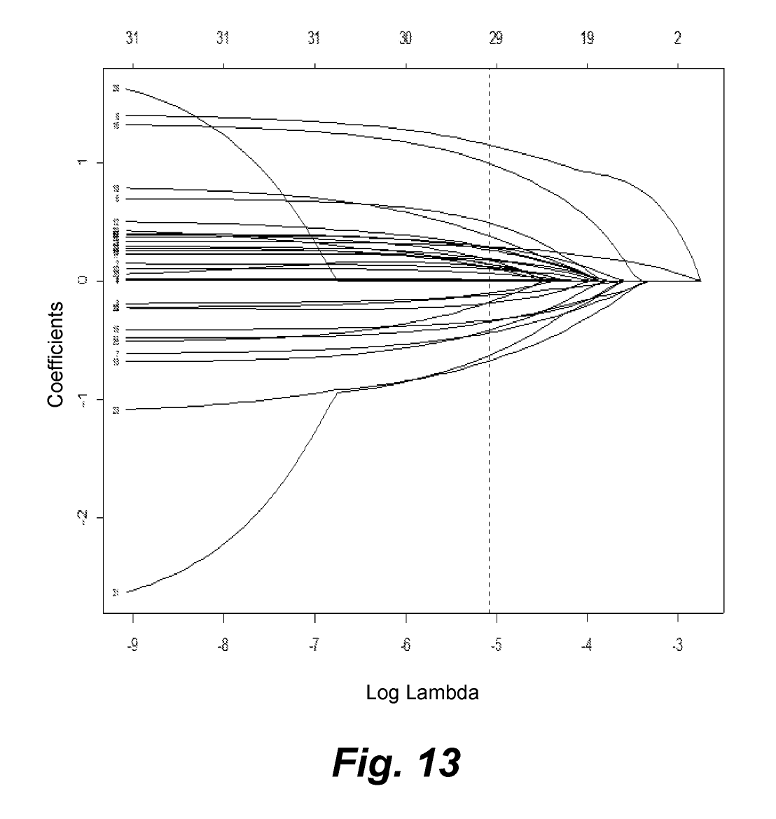

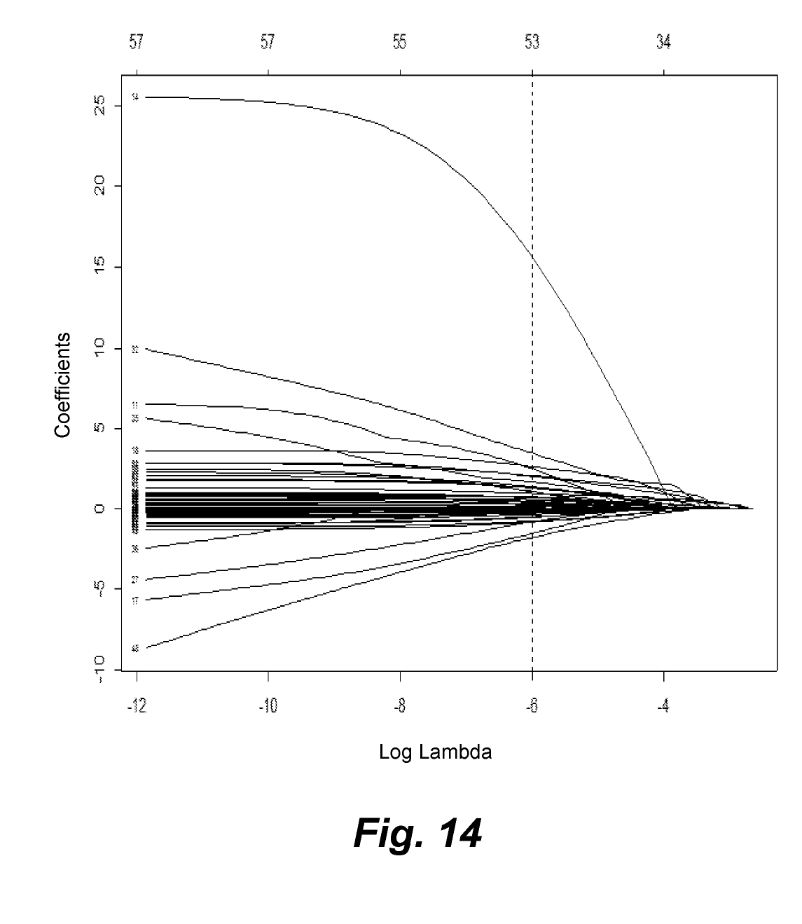

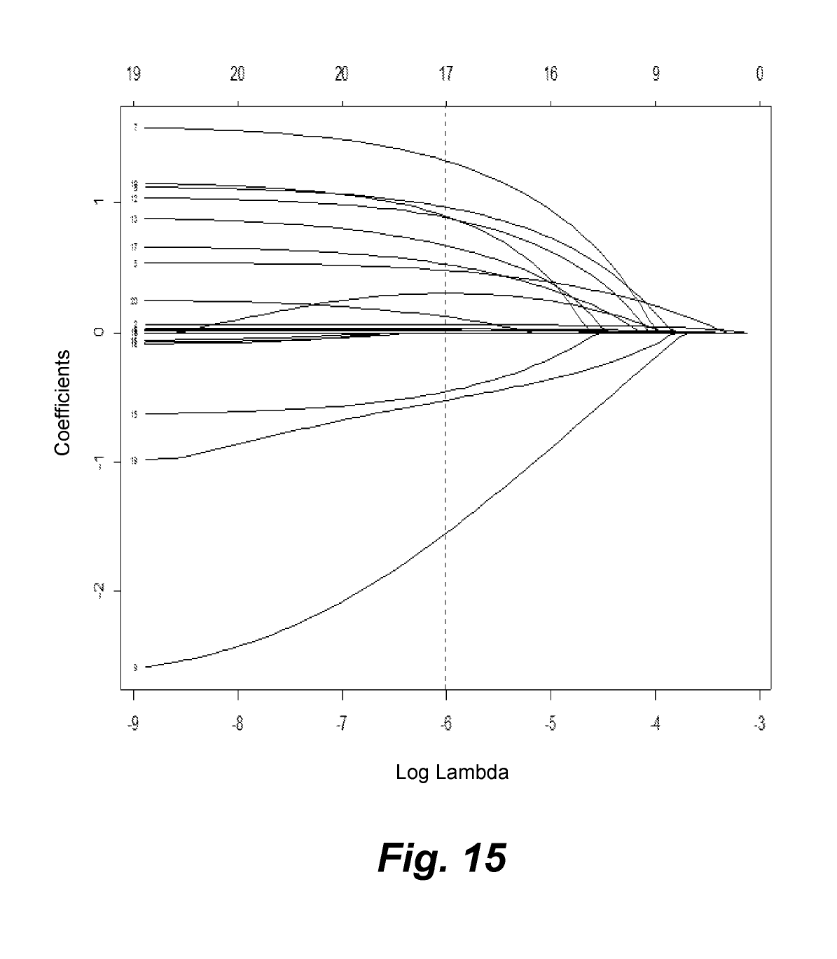

The following figures show the variable shrinkage pathway for different values of λ, for PE, SPTB, SGA, and GDM models. Each predictor is represented by a line, and the distance between the coefficients indicates their correlation (i.e. lines that are closer together indicate a stronger correlation). As expected, the coefficients of predictors will eventually shrink towards zero. FIG. 9 shows PE model (tier 2) variable shrinkage pathway. FIG. 10 shows SPTB model (tier 1) variable shrinkage pathway. FIG. 11 shows SPTB model (tier 2) variable shrinkage pathway. FIG. 12 shows SGA model (tier 1) variable shrinkage pathway. FIG. 13 shows SGA model (tier 2) variable shrinkage pathway. FIG. 14 shows GDM model (tier 1) variable shrinkage pathway. FIG. 15 shows GDM model (tier 2) variable shrinkage pathway.

Model Development

After variable shrinkage was performed for each of the complications, the chosen sets of variables were then included in Logistic regression for modelling of the odds of PE, SPTB, SGA, and GDM. A model for each tier was developed separately for each complication, with specific requirements for each tier.

Tiered Model Specifications

Individual models developed at each tier were adjusted to accommodate the specific needs, and their predicted risks were then later integrated with the subsequent tiers to provide an overall risk classification.

With the first tier as initial screening, a higher sensitivity was preferred, as the main purpose of this tier was to identify all women who may be at risk. At this stage, the prediction was based on predictors available at first antenatal visit (for SCOPE, at 15 weeks of gestation), which includes current dietary practice, pre-existing health conditions, family history, as well as clinical measurements such as blood pressure.

For the second tier prediction which can be performed on or prior to 20 weeks of gestation, a high positive predictive value (PPV), i.e. low false positive rates, was preferred to minimize the chance of unnecessary interventions. Predictors at this tier may include SNPs or details of ultrasound scan.

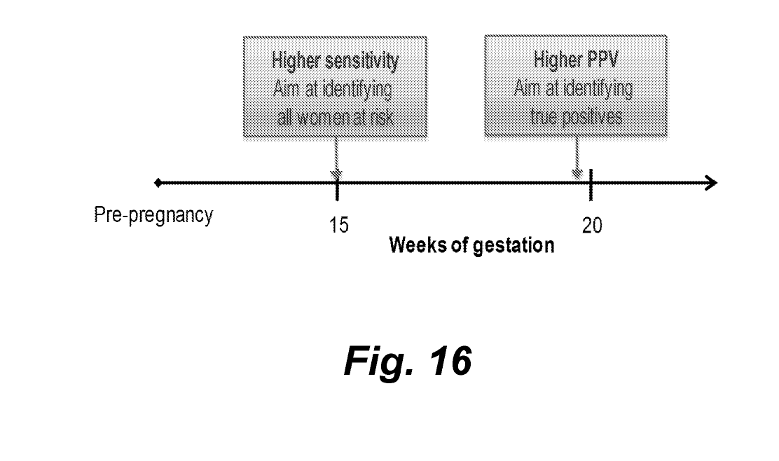

FIG. 16 shows tiered model specifications.

The individual models for tiers 1 and 2, described above, were developed using penalized logistic regression, with the best model selected based on penalty functions and accuracy measures, and then integrated by calculating the post-test odds using Bayes' theorem at each stage of pregnancy. The predicted risk was then further classified into 3 classes (low, moderate, and high risk).

A major advantage of a tiered approach was that risk estimates or prediction can be obtained throughout pregnancy, which allows constant monitoring and update of predicted risk for individuals when new predictors are available or when conditions change, and hence, the level of care may be tailored for individual women. In addition, having the first tier with a high sensitivity at first visit assures that the proportion of disease amongst women predicted at low risk at tier 1 is lower than those predicted at risk. This means that by 15 weeks of gestation, the first group of low-risk women can be identified and continue regular antenatal visits, while those identified at risk may go through further screening at tier 2 and may be recommended for tailored care.

Theoretical Calculations

In binary logistic regression, the response variable, denoted by Y, can take only two values: 0 or 1, which represents ‘not at risk’ or ‘at risk’. Logistic regression classifies variables into the two groups (i.e. group 1 and 0) by modeling the posterior probability of class 1 membership via a linear function of the explanatory variables.

Instead of directly modeling the posterior probability of class 1 membership, p=E[Y|X], the logit transformation was used. This ensures the predicted response from the linear regression was bounded between 0 and 1.

Let X₁, X₂, . . . , Xₖ denotes the k explanatory variables. Then the logit transformation of p, which is defined as the logarithm of the odds ratio, is modeled by the linear function:

$$logit ( p ) = log ( \frac{p}{1 - p} ) = {β}_{0} + {β}_{1} {X}_{1} + {β}_{2} {X}_{2} + … + {β}_{k} {X}_{k}$$

It is convenient to express the linear regression function in matrix form β^{T}X, where β=[β₀, β₁, . . . , βₖ]^{T} and X=[1, X₁, X₂, . . . , Xₖ]^{T}. The probability of the outcome of interest can then be written as:

$$p = \frac{1}{1 + {e}^{- {β}^{T} X}}$$

Program Code for Model Development

The glm function included in R base installation was used to fit the Logistic regression models. For ROC curve plotting, the ROCR package (Sing T, Sander O, Beerenwinkel N and Lengauer T (2005) ROCR: visualizing classifier performance in R. Bioinformatics, 21(20), pp 7881) was used. The cross-validation statistics were calculated using the boot package (Canty A and Ripley B (2015) boot: Bootstrap R (S-Plus) Functions. R package version 1.3-17; Davison A and Hinkley D (1997) Bootstrap Methods and Their Applications. Cambridge University Press, Cambridge. ISBN 0-521-57391-2).

Training Logistic Regression

Due to the large number of models to be fitted with different combinations of predictors, a wrapper function custom.LR was written. This function parses a matrix of chosen predictors (choosen.vars) and fed it into the glm function with logit link for logistic regression. It then returns a glm object.

[R] Code Snippet 3.3.1: custom.LR

custom.LR <− function(choosen.vars,dependent){

name1 <− choosen.vars[1]
for(nam in 2:length(choosen.vars)){

name1 <− paste(name1,“+”,choosen.vars[nam],sep=“”)

}
LR.cust.T <− paste(“LR.cust <− glm(“,dependent,“ ~ ”,name1,”

,family=binomial(logit))“,sep

=“”)

eval(parse(text=LR.cust.T))

}

The estimated coefficients can then be accessed from the stored glm object. Further model selection through AIC can then be performed.

[R] Code Snippet 3.3.2: Logistic regression

LR1 <− custom.LR(choose.names,“f39_pet”) #for PE models
LR1.sum <− summary(LR1)
LR1.best <− stepAIC(LR1) #stepwise with AIC
LR1.best.sum <− summary(LR1.best)

An odds function was also written to calculate the odds and its corresponding 95% confidence intervals. This function extracts the model coefficients and error estimates from the fitted logistic regression, and then calculates the upper and lower confidence intervals using 95% CI: exp(\hat{β}±z_{α/2} SE[\hat{β}]), where α=0.05.

[R] Code Snippet 3.3.3: odds

odds <− function(LR){

LR.coef <− LR$coefficients
if(nrow(LR.coef)<2){odds.tab <− data.frame(“Odds”=1,“Lower 95%

CI”=NA,“Upper 95% CI”,NA, check.names=FALSE)}

if(nrow(LR.coef )==2){

LR.stats <− LR$coefficients[2,]
LR.odds.upper <−
exp(LR.stats[1]+(qnorm(1-0.025)*LR.stats[2]))
LR.odds.lower <−
exp(LR.stats[1]−(qnorm(1-0.025)*LR.stats[2]))
odds.tab <− data.frame(“Odds”=exp(LR.stats[1])

,“Lower 95%

CI”=LR.odds.lower

,“Upper 95% CI”=LR.odds.upper

,check.names=FALSE)

}
if(nrow(LR.coef)>2){

LR.stats <− LR$coefficients[2:nrow(LR$coefficients),]
LR.odds.upper <−

exp(LR.stats[,1]+(qnorm(1-0.025)*LR.stats[,2]))

LR.odds.lower <−

exp(LR.stats[,1]−(qnorm(1-0.025)*LR.stats[,2]))

odds.tab <− data.frame(“Odds”=exp(LR.stats[,1])

,“Lower 95%

CI”=LR.odds.lower

,“Upper 95% CI”=LR.odds.upper

,check.names=FALSE)

}
return(odds.tab)

}

Calculating Accuracy Measures

Given a 2×2 matrix of observed true cases of outcome vs. predicted cases, the sensitivity and specificity can then be calculated. The Xtab function was written to construct the 2×2 matrix from the fitted model, and the resulting matrix can then be parsed to the rs function to calculate the sensitivity and specificity.

Basically, the Xtab function first obtains the predicted probabilities using predict function from glm, and then dichotomize the predicted probabilities into 0 or 1 based on a chosen threshold (cut-off). It then cross-tabulates the dichotomized result with the observed cases of the outcome of interest (dependent).

An additional swap option is added to the Xtab function for convenient manipulation of the 2×2 matrix. This option swaps the columns and rows as follows:

```

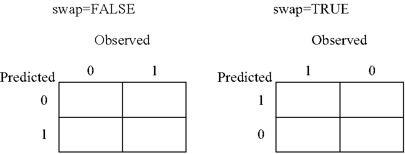

[R] Code Snippet 3.3.4: Xtab
Xtab <- function (LR, cutoff=0.5, dependent, swap=FALSE) {
  LR.pred <- predict(LR, type=″response″)
  if(swap==TRUE) {cutoff <- 1-cutoff}
  LR.pred[LR.pred<cutoff] <- 0
  LR.pred[LR.pred>=cutoff] <- 1
  Xtab.freq <- table(″Predicted″=LR.pred, ″Observed″=dependent)
  if(swap==TRUE) {
    Xtab.freq[, c(1, 2)] <- Xtab.freq[, c(2, 1)]
    Xtab.freq[c(1, 2),] <- Xtab.freq[c(2, 1),]
  }
  csum <-
c(Xtab.freq[1, 1]+Xtab.freq[2, 1], Xtab.freq[1, 2]+Xtab.freq[2, 2])
  Xtab.mod1 <- rbind(Xtab.freq, ″Total″=csum)
  rsum <-
c(Xtab.mod1 [1, 1]+Xtab.mod1 [1, 2], Xtab.mod1 [2, 1]+Xtab.mod1
[2, 2], Xtab.mod1 [3, 1]+Xtab.mod1 [3, 2])
  Xtab <- cbind(Xtab.mod1, ″Total″=rsum)
  dep.tab <- table(dependent)
  pred.tab <- table(LR.pred)
  colnames(Xtab) <-
c(paste(″Observed(″, rownames (dep.tab) [1],″)″, sep=″ ″),
paste(″Observed(″, rownames (dep.tab) [2],″)″, sep=″ ″), ″Total″)
  rownames(Xtab) <-
c(paste(″Predicted(″, rownames (pred.tab) [1],″)″, sep=″ ″),
paste(″Predicted(″, rownames (pred.tab) [2],″)″, sep=″ ″), ″Total″)
  if(swap==TRUE) {
  colnames(Xtab) <-
c(paste(″Observed(″, rownames (dep.tab) [2],″)″, sep=″ ″),
paste(″Observed(″, rownames (dep.tab) [1],″)″, sep=″ ″), ″Total″)
  rownames(Xtab) <-
c(paste(″Predicted(″, rownames (pred.tab) [2],″)″, sep=″ ″),
paste(″Predicted(″, rownames (pred.tab) [1],″)″, sep=″ ″), ″Total″)
  }
    Xtab}
```

The rs function then calculates the sensitivity and specificity as follows:

$$Sensitivity r = \frac{a}{a + c} Specificity s = \frac{d}{b + d} Overall o = \frac{a + d}{a + b + c + d}$$

[R] Code Snippet 3.3.5: rs

rs <− function(Xtab){

r <− Xtab[1,1]/Xtab[3,1]
s <− Xtab[2,2]/Xtab[3,2]
o <− (Xtab[1,1]+Xtab[2,2])/Xtab[3,3]
rs <− data.frame(“Sensitivity”=r,“Specificity”=s,“Overall”=o)

}

The workflow for calculating accuracy measures are as follows:

[R] Code Snippet 3.3.6: Accuracy measures

LR <- LR1
cut1 <- 0.95 #desired cut-point
LR.Xtab <- Xtab(LR,cut1,f39_pet,swap=TRUE) #for PE models
LR.rs <- rs(LR.Xtab)
LR.rs #table of accuracy measures

Assessing Predictive Value

An ROC curve and cross-validation statistics was then used to obtain an estimate of prediction error of the model on unseen data.

To plot an ROC curve, the predicted probabilities must be obtained first (LR1.pred). The true positive and false positive rates are then calculated using the performance function. The true positive rates are then plotted against false positive rates to create an ROC curve. An additional diagonal reference line was also added to indicate separation from random predictions (FIG. 23).

The estimated prediction error from cross-validation statistics was calculated using cv.glm function in the boot package as shown in F:

[R] Code Snippet 3.3.8: Cross-validation

library(boot)
cost <- function(r, pi=0) mean(abs(r-pi)>0.5)
cvK <- cv.glm(dat,LR,cv,K=10) #10-fold CV
print(cvK$delta)

Individual Models

There are 2 models for each outcome of interest (one for each tier). Model performance measures included sensitivity, specificity, AUC, and cross-validation statistics are also shown.

Preeclampsia Model (Tier 1)

The input variables are shown in Table 5. The probability threshold for Tier 1 is 0.02.

TABLE 5
PE model (tier 1) estimates

| Predictors | Input |
|---|---|
| (intercept) | no input needed |
| Maternal age | number |
| Mean arterial pressure | number |
| BMI | number |
| Family history of PE | No = 0 |
| | Yes = 1 |
| Family history of chronic hypertension | No = 0 |
| | Yes = 1 |
| Participant's birthweight | Number |
| Any vaginal bleeding continuing for at least 5 days | No = 0 |
| | Yes = 1 |
| Any previous miscarriage at <=10 wks gestation | No = 0 |
| with same man who has fathered the current | Yes = 1 |
| pregnancy | |
| Duration of sex without contraception with father of | number |
| baby before current pregnancy >12 months | |
| Frequency consumed fruit in the month prior to | 3-6x per week = 1 |
| conception | otherwise = 0 |
| | 1-2x per week = 1 |
| | otherwise = 0 |
| | 1-3x per month or |
| | less = 1 |
| | otherwise = 0 |
| units of alcohol per week in the 1st trimester | number |
| number of cigarettes per day | number |

Preeclampsia Model (Tier 2)

The input variables are shown in Table 6. Using a probability threshold of 0.1, the positive and negative likelihood ratios are 3.081 and 0.597 respectively.

TABLE 6
PE model (tier 2) estimates

| Predictors | Input |
|---|---|
| (intercept) | no input needed |
| Mean arterial pressure | number |
| BMI | number |
| Family history of PE | No = 0 |
| | Yes = 1 |
| Participant's birthweight | number |
| Any previous miscarriage at <=10 wks gestation | No = 0 |
| with same man who has fathered the current | Yes = 1 |
| pregnancy | |
| Months to conceive | ≤12 mths = 0 |
| | >12 mths = 1 |
| Frequency consumed fruit in the month prior to | 3-6x per week = 1 |
| conception | otherwise = 0 |
| | 1-2x per week = 1 |
| | otherwise = 0 |
| | 1-3x per month or |
| | less = 1 |
| | otherwise = 0 |
| units of alcohol per week in the 1st trimester | number |
| number of cigarettes per day | number |
| Mum_AGT_RS4762 | TT = 1 |
| | CC/CT = 0 |
| Mum_AGTR1_RS5186 | CC = 1 |
| | AA/AC = 0 |
| Mum_IL10_RS1800896 | GG/GA = 1 |
| | AA = 0 |
| Part_HIF1a_RS11549465 | TT/TC = 1 |
| | CC = 0 |
| Part_NOS2A_RS1137933 | TT/TC = 1 |
| | CC = 0 |
| Part_TP53_RS1042522 | GG/GC = 1 |
| | CC = 0 |
| Mum_MTHFR_RS1801131 | CC = 1 |
| | AA/AC = 0 |
| Part_GSTP1_RS1695 | GG = 1 |
| | AA/AG = 0 |
| Part_MTRR_RS1801394 | GG/GA = 1 |
| | AA = 0 |
| Mum_TGFB_rs1800469 | GG = 1 |
| | AA/AG = 0 |
| Part_TGFB_rs1800469 | GG/GA = 1 |
| | AA = 0 |
| Mum_PGF_RS1042886 | CC = 1 |
| | AA/AC = 0 |
| Part_PGF_RS1042886 | CC = 1 |
| | AA/AC = 0 |
| Part_CYP11A1_RS8039957 | GG/GA = 1 |
| | AA = 0 |
| Mum_INSR_RS2059806 | GG/GA = 1 |
| | AA = 0 |
| Part_MMP2_RS243865 | TT = 1 |
| | CC/CT = 0 |

Spontaneous Preterm Birth Model (Tier 1).

The input variables are shown in Table 7. The probability threshold for Tier 1 is 0.005.

TABLE 7
SPTB model (tier 1) estimates

| Predictors | Input |
|---|---|
| (intercept) | no input needed |
| Maternal height | number |
| Yrs of schooling | number |
| Gravidity | number |
| Months to conceive | number |
| Folate dose per day in 1st trimester | No = 0 |
| | <=800 μg = 1 |
| | >800 μg = 1 |
| | otherwise = 0 |
| Frequency consumed fruit in pregnancy at 15 wks | 3-6x per week = 1 |
| | otherwise = 0 |
| | 1-2x per week = 1 |
| | otherwise = 0 |
| | 1-3x per month = 1 |
| | otherwise = 0 |
| | Never = 1 |
| | otherwise = 0 |
| Extreme exercise in pregnancy (undertook vigrous | No = 0 |
| exercise at least once a day) | Yes = 1 |
| Number of times climbed stairs in the last month | <10x/day = 1 |
| | otherwise = 0 |
| | >=10x/day = 1 |
| | otherwise = 0 |
| If you do paid work, what activity best describes the | Sitting and some |
| main activities at work | walking = 1 |
| | otherwise = 0 |
| | Standing = 1 |
| | otherwise = 0 |
| | Standing/ |
| | walking = 1 |
| | otherwise = 0 |
| | Standing/walking/ |
| | intermittent |
| | exercise = 1 |
| | otherwise = 0 |
| | Regular exercise = 1 |
| | otherwise = 0 |
| Current work situation | Part time work = 1 |
| | otherwise = 0 |
| | Student = 1 |
| | otherwise = 0 |
| | Homemaker = 1 |
| | otherwise = 0 |
| | Unemployed = 1 |
| | otherwise = 0 |
| | Sickness |
| | beneficiary = 1 |
| | otherwise = 0 |
| State-Trait Anxiety Inventory | <=90th centile = 0 |
| | >90th centile = 1 |
| I have felt better than ever in pregnancy | Rarely = 1 |
| | otherwise = 0 |
| | Some days = 1 |
| | otherwise = 0 |
| | Most days = 1 |
| | otherwise = 0 |
| | Every day = 1 |
| | otherwise = 0 |
| Any hospital admissions due to hyperemesis | No = 0 |
| | Yes = 1 |
| Participant's immigration history | Participant not |
| | immigrant and |
| | family |
| | history |
| | unknown = 1 |
| | otherwise = 0 |
| | 1 parent |
| | immigrated = 1 |
| | otherwise = 0 |
| | Both parents |
| | immigrated = 1 |
| | otherwise = 0 |
| | Participant |
| | immigrated = 1 |
| | otherwise = 0 |
| Number of Lletz treatments | 1 Rx = 1 |
| | otherwise = 0 |
| Donor sperm or donor egg used in this | No = 0 |
| pregnancy | Fertility Rx |
| | partner = 1 |
| | Fertility Rx |
| | donor = 1 |
| | otherwise = 0 |
| Any sister had a history of LBW baby | No = 0 |
| | Yes = 1 |
| Family history of LBW baby, i.e. participant's | No = 0 |
| mother or sister had had LBW baby | Yes = 1 |
| participant's mother has had a CVA | No = 0 |
| | Unknown = 1 |
| | Yes = 1 |
| | otherwise = 0 |
| Participant's mother had a history of PET | No = 0 |
| | Unknown = 1 |
| | had PET 1x |
| | otherwise = 0 |
| | had PET >=2x |
| | otherwise = 0 |
| Participant's mother had a history of pregnancy | No/unknown = 0 |
| induced hypertension | PIH 1x = 1 |
| | PIH >=2x |
| | otherwise = 0 |
| Mum_AGT_RS4762 | TT = 1 |
| | CC/CT = 0 |
| Mum_BCL2_RS2279115 | CC/CA = 1 |
| | AA = 0 |
| Mum_MBL2_RS1800450 | GG/GA = 1 |
| | AA = 0 |
| Mum_TCN2_RS1801198 | GG/GC = 1 |
| | CC = 0 |
| Mum_FLT1_FLT1C677T | TT/TC = 1 |
| | CC = 0 |
| Mum_IGF2R_RS2274849 | GG = 1 |
| | AA/AG = 0 |
| Mum_IL1B_RS16944 | GG = 1 |
| | AA/AG = 0 |
| Mum_uPA_RS2227564 | TT = 1 |
| | CC/CT = 0 |
| Mum_CYP11A1_RS4887139 | GG/GA = 1 |
| | AA = 0 |
| Mum_CYP11A1_RS8039957 | GG/GA = 1 |
| | AA = 0 |
| Mum_IGF1R_RS11247361 | GG/GC = 1 |
| | CC = 0 |
| Mum_MMP2_RS243865 | TT/TC = 1 |
| | CC = 0 |
| Mum_MMP9_rs3918242 | TT/TC = 1 |
| | CC = 0 |
| Mum_TIMP3_RS5749511 | TT/TC = 1 |
| | CC = 0 |

Spontaneous Preterm Birth Model (Tier 2)

The input variables are shown in Table 8. Using a probability threshold of 0.2, the positive and negative likelihood ratios are 3.296 and 0.555 respectively.

TABLE 8
SPTB model (tier 2) estimates

| Predictors | Input |
|---|---|
| (intercept) | no input needed |
| Months to conceive | number |
| Transvaginal cervical length | number |
| Folate dose per day in 1st trimester | No = 0 |
| | <=800 μg = 1 |
| | >800 μg = 1 |
| | otherwise = 0 |
| Extreme exercise in pregnancy (undertook vigrous | No = 0 |
| exercise at least once a day) | Yes = 1 |
| Number of times climbed stairs in the last month | <10x/day = 1 |
| | otherwise = 0 |
| | >=10x/day = 1 |
| | otherwise = 0 |
| State-Trait Anxiety Inventory | <=90th centile = 0 |
| | >90th centile = 1 |
| I have felt better than ever in pregnancy | Rarely = 1 |
| | otherwise = 0 |
| | Some days = 1 |
| | otherwise = 0 |
| | Most days = 1 |
| | otherwise = 0 |
| | Every day = 1 |
| | otherwise = 0 |
| Participant's immigration history | Participant not |
| | immigrant and family |
| | history |
| | unknown = 1 |
| | otherwise = 0 |
| | 1 parent |
| | immigrated = 1 |
| | otherwise = 0 |
| | Both parents |
| | immigrated = 1 |
| | otherwise = 0 |
| | Participant |
| | immigrated = 1 |
| | otherwise = 0 |
| Number of Lletz treatments | 1 Rx = 1 |
| | otherwise = 0 |
| Family history of LBW baby, i.e. participant's | No = 0 |
| mother or sister had LBW baby | Yes = 1 |
| Participant's mother has had a CVA | No = 0 |
| | Unknown = 1 |
| | Yes = 1 |
| | otherwise = 0 |
| Participant's mother had a history of PET | No = 0 |
| | Unknown = 1 |
| | had PET 1x = 1 |
| | otherwise = 0 |
| | had PET >=2x = 1 |
| | otherwise = 0 |
| Participant's mother had a history of pregnancy | No/unknown = 0 |
| induced hypertension | PIH 1x = 1 |
| | PIH >=2x |
| | otherwise = 0 |
| Mum_AGT_RS4762_TTT | TT = 1 |
| | CC/CT = 0 |
| Mum_MBL2_RS1800450_AAGGA | GG/GA = 1 |
| | AA = 0 |
| Rachael_Mum_IGF2R_RS2274849_GGG | GG = 1 |
| | AA/AG = 0 |
| Rachael_Mum_uPA_RS2227564_TTT | TT = 1 |
| | CC/CT = 0 |
| Steve_Mum_IGF1R_RS11247361_CCGGC | GG/GC = 1 |
| | CC = 0 |
| Steve_Mum_MMP2_RS243865_CCTTC | TT/TC = 1 |
| | CC = 0 |
| Steve_Mum_TIMP3_RS5749511_CCTTC | TT/TC = 1 |
| | CC = 0 |

Small for Gestational Age Model (Tier 1)

The input variables are shown in Table 9. The probability threshold for Tier 1 is 0.04.

TABLE 9
SGA model (tier 1) estimates

| Predictors | Input |
|---|---|
| (intercept) | no input needed |
| Total number of cigarettes a woman was exposed to | number |
| in the 1st trimester | |
| Diastolic blood pressure | number |
| Head circumference (cm) | number |
| Consumed/inhaled/injected other recreational drugs | No = 0 |
| or binge alcohol consumption (>=6 units/session) | Yes = 1 |
| Low (<3x times/mth) fruit consumption in the month | No = 0 |
| prior to conception | Yes = 1 |

Small for Gestational Age Model (Tier 2). The input variables are shown in Table 10. Using a probability threshold of 0.3, the positive and negative likelihood ratios are 4.937 and 0.837 respectively.

TABLE 10
SGA model (tier 2) estimates

| Predictors | Input |
|---|---|
| (intercept) | no input needed |
| Head circumference (cm) | number |
| Diastolic blood pressure | number |
| Mean arterial pressure | number |
| Use of barrier contraception (condoms or | No = 0 |
| diaphragm) with biological father of baby | Yes = 1 |
| Consumed/inhaled/injected other recreational drugs | No = 0 |
| or binge alcohol consumption (>=6 units/session) | Yes = 1 |
| Smoking status | Never smoked = 0 |
| | Smoked pre-preg, |
| | but quit smoking |
| | before |
| | pregnant = 1 |
| | Smoked in preg, |
| | but quit smoking |
| | before 1st |
| | visit = 1 |
| | otherwise = 0 |
| | Smoking at 15 |
| | wks = 1 |
| | otherwise = 0 |
| Frequency consumed fruit in the month | >=1x per day = 0 |
| prior to conception | 3-6x per week = 1 |
| | 1-2x per week = 1 |
| | otherwise = 0 |
| | 1-3x per month or |
| | less = 1 |
| | otherwise = 0 |
| Rhesus factor | Rh Positive = 0 |
| | Rh Negative = 1 |
| Mum_IL6_RS1800795 | GG/GC = 1 |
| | CC = 0 |
| Mum_F2_RS1799963 | TT/TC = 1 |
| | CC = 0 |
| Mum_NAT1_RS1057126 | TT = 1 |
| | AA/AT = 0 |
| Part_NAT1_RS1057126 | TT = 1 |
| | AA/AT = 0 |
| Part_TCN2_RS1801198 | GG = 1 |
| | CC/CG = 0 |
| Mum_INS_rs3842752 | TT/TC = 1 |
| | CC = 0 |
| Part_THBS1_RS2228262 | GG/GA = 1 |
| | AA = 0 |
| Part_IGF2AS_RS1004446 | TT = 1 |
| | CC/CT = 0 |

Gestational Diabetes Mellitus Model (Tier 1)

The input variables are shown in Table 11. The probability threshold for Tier 1 is 0.00001.

TABLE 11
GDM model (tier 1) estimates

| Predictors | Input |
|---|---|
| (intercept) | no input needed |
| Folate dose (μg per day) in 1st trimester | number |
| Folate dose (μg per day) at 15 wks | number |
| Diastolic blood pressure | number |
| BMI | number |
| Height (cm) | number |
| Pulse per minute | number |
| Random glucose (mmol/L) measured by | number |
| glucometer at 15 wks | |
| Waist (cm) | number |
| Mean arterial pressure | number |
| Paternal age | number |
| Participant's booking Haematocrit (PCV) | number |
| Yrs of schooling | number |
| Participant's birthweight | number |
| Fertility treatment to conceive current pregnancy | No = 0 |
| | Yes = 1 |
| Any previous terminations at >10 weeks | No = 0 |
| | Yes = 1 |
| Hormonal treatment, other than clomiphene, to | No = 0 |
| assist conception of current pregnancy | Yes = 1 |
| Time of last colposcopy before conception of | No/unknown = 1 |
| current pregnancy | otherwise = 0 |
| | 7-12 mths = 1 |
| | otherwise = 0 |
| | >12 mths = 1 |
| | otherwise = 0 |
| Received fertility treatment for PCOS prior to/at | No = 0 |
| conception | Yes = 1 |
| Participant's father has type 2 diabetes | No = 0 |
| | Yes = 1 |
| Participant's father has diabetes type not | No = 0 |
| specified | Yes = 1 |
| Family history of diabetes type 2 (Participant's | No = 0 |
| mother, father, sibling) | Yes = 1 |
| Participant's mother had a history of PET | No = 0 |
| | Unknown = 1 |
| | had PET 1x = 1 |
| | otherwise = 0 |
| | had PET >=2x = 1 |
| | otherwise = 0 |
| Bleeding gums when brushing teeth at 15 wks | No/uknown = 0 |
| | Yes = 1 |
| Units of alcohol per week in the 1st | No = 0 |
| trimester | 1 to 2 = 1 |
| | 3 to 7 = 1 |
| | otherwise = 0 |
| | 8 to 14 = 1 |
| | otherwise = 0 |
| | >14 = 1 |
| | otherwise = 0 |
| High (>=3 times per day) fruit consumption | No = 0 |
| in the month prior to conception | Yes = 1 |
| High (>=3 times per day) fruit consumption | No = 0 |
| at 15 wks | Yes = 1 |
| Any proteinuria at 15 wks | dipstick = trace or |
| | negative or urinary |
| | PCR measurement |
| | <30 mg/mmol = 0 |
| | dipstick>=1+ or |
| | or urinary PCR |
| | measurement |
| | >=30 mg/mmol = 1 |
| Number of times climbed stairs in the last | Never = 0 |
| month | <10x/day = 1 |
| | >=10x/day = 1 |
| | otherwise = 0 |
| In last month, number of episodes of waking | None = 0 |
| during a night's sleep | Once = 1 |
| | 2-3 times = 1 |
| | otherwise = 0 |
| | >=4 times |
| | otherwise = 0 |
| Snored most nights | No = 0 |
| | Yes = 1 |
| Support people around to provide emotional | All the time = 0 |
| support | Most of the time = 1 |
| | Sometimes = 1 |
| | otherwise = 0 |
| | Seldom/Never = 1 |
| | otherwise = 0 |

Gestational Diabetes Mellitus Model (Tier 2)

The input variables are shown in Table 12. Using a probability threshold of 0.3, the positive and negative likelihood ratios are 6.9198 and 0.579 respectively.

TABLE 12
GDM model (tier 2) estimates

| Predictors | Input |
|---|---|
| (intercept) | no input needed |
| Folate dose (μg per day) in 1st trimester | number |
| BMI | number |
| Height (cm) | number |
| Diastolic blood pressure | number |
| Pulse per minute | number |
| Yrs of schooling | number |
| Duration of sex without contraception before | number |
| conception with father of baby | |
| Any previous terminations at >10 weeks | No = 0 |
| | Yes = 1 |
| Participant's father has type 2 diabetes | No = 0 |
| | Yes = 1 |
| High (>=3 times per day) fruit consumption in | No = 0 |
| the month prior to conception | Yes = 1 |
| Mum_AGT_RS4762_TTT | TT = 1 |
| | CC/CT = 0 |
| Mum_FTO_RS9939609_TTT | TT = 1 |
| | AA/AT = 0 |
| Mum_NOS2A_RS1137933_TTT | TT = 1 |
| | CC/CT = 0 |
| Mum_PTEN_rs2673832_GGG | GG = 1 |
| | AA/AG = 0 |
| Mum_CYP24A1_RS2248137_CCGGC | GG/GC = 1 |
| | CC = 0 |
| Mum_XRCC2_RS3218536_GGG | GG = 1 |
| | AA/AG = 0 |
| Mum_ANGPT1_rs2507800_AATTA | TT/TA = 1 |
| | AA = 0 |
| Mum_KDR_RS2071559_TTT | TT = 1 |
| | CC/CT = 0 |
| Mum_CYP11A1_RS4887139_AAGGA | GG/GA = 1 |
| | AA = 0 |
| Mum_H19_RS2839701_GGG | GG = 1 |
| | CC/CG = 0 |

Risk Integration and Classification

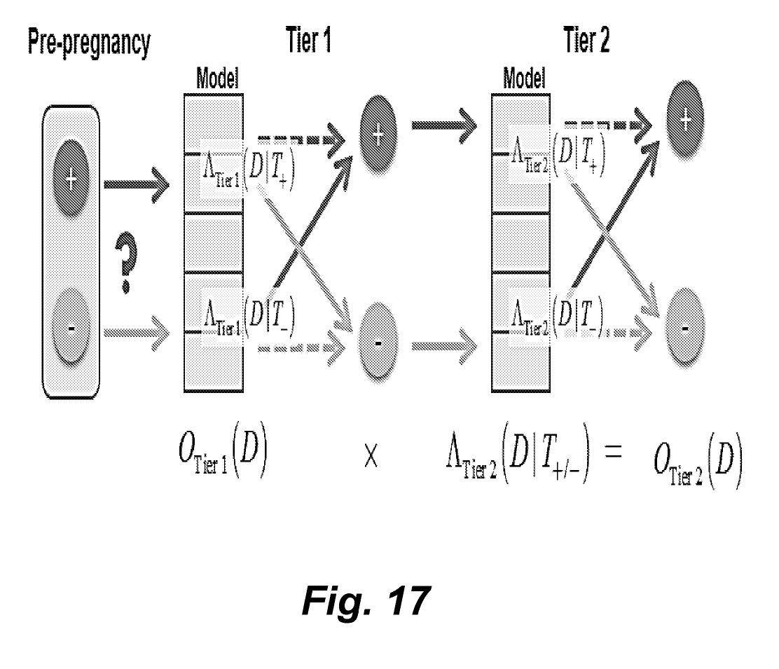

After individual models were obtained for each tier, the final process of model development was to integrate risk predictions from all tiers to perform a process of elimination, which assisted in stratifying the level of care for individual patients. This can be achieved, for example, by applying the Bayes' theorem to obtain a post-test odds of tier 2 based on prior ‘guess’ obtained from the predicted risk of tier 1 and the likelihood ratio of tier 2 individual model (FIG. 17).

Theoretical Calculations

With the application of Bayes' theorem, an adjusted predicted probability or post-test probability of disease that incorporates a pre-test probability can be obtained. This is a useful tool to “rule in” and “rule out” a disease for an individual. By Bayes' theorem, the post-test probability is given by:

$$P ( D \vert {T}_{+ / -} ) = \frac{P ( D ) P ( {T}_{+ / -} \vert D )}{P ( {T}_{+ / -} \vert D ) P ( D ) + P ( {T}_{+ / -} \vert \overline{D} ) P ( \overline{D} )}$$

Since P(D) can be converted into odds O(D) using:

$$P ( D ) = \frac{O ( D )}{1 + O ( D )}$$

Hence, the post-test probability can be expressed as:

$$P ( D \vert {T}_{+ / -} ) = \frac{\frac{O ( D ) P ( {T}_{+ / -} \vert D )}{1 + O ( D )}}{\frac{O ( D ) P ( {T}_{+ / -} \vert D )}{1 + O ( D )} + ( 1 - \frac{O ( D )}{1 + O ( D )} P ( {T}_{+ / -} \vert \overline{D} ) )}$$

$$\frac{O ( D \vert {T}_{+ / -} )}{1 + O ( D \vert {T}_{+ / -} )} = \frac{O ( D ) P ( {T}_{+ / -} \vert D )}{O ( D ) [ P ( {T}_{+ / -} \vert D ) + P ( {T}_{+ / -} \vert \overline{D} ) ]}$$

$$\frac{O ( D \vert {T}_{+ / -} )}{1 + O ( D \vert {T}_{+ / -} )} = \frac{1}{1 + ( \frac{P ( {T}_{+ / -} \vert \overline{D} )}{O ( D ) P ( {T}_{+ / -} \vert D )} )}$$

$$O ( D \vert {T}_{+ / -} ) = 1 + O ( D \vert {T}_{+ / -} ) - [ O ( D \vert T ) \frac{P ( {T}_{+ / -} \vert D )}{O ( D ) P ( {T}_{+ / -} \vert D )} ]$$

This is also known as the odds form:

$$O ( D \vert {T}_{+ / -} ) = O ( D ) \frac{P ( {T}_{+ / -} \vert D )}{P ( {T}_{+ / -} \vert \overline{D} )}$$

Hence, the integrated post-test odd after Tier 2, with pre-test odds obtained from Tier 1, is given by:

O_{Tier 2}(D|T_{+/-})=O_{Tier 1}(D)·Λ_{Tier 2}(D|T_{+/-})
where

$${Λ}_{Tier 2} ( D \vert {T}_{+ / -} ) = \frac{P ( {T}_{+ / -} \vert D )}{P ( {T}_{+ / -} \vert \overline{D} )}$$

is positive or negative likelihood ratio of the Tier 2 model. They are given by

$${Λ}_{Tier 2} ( D \vert {T}_{+} ) = \frac{r}{1 - s} and {Λ}_{Tier 2} ( D \vert {T}_{-} ) = \frac{1 - r}{s} .$$

Risk Classification

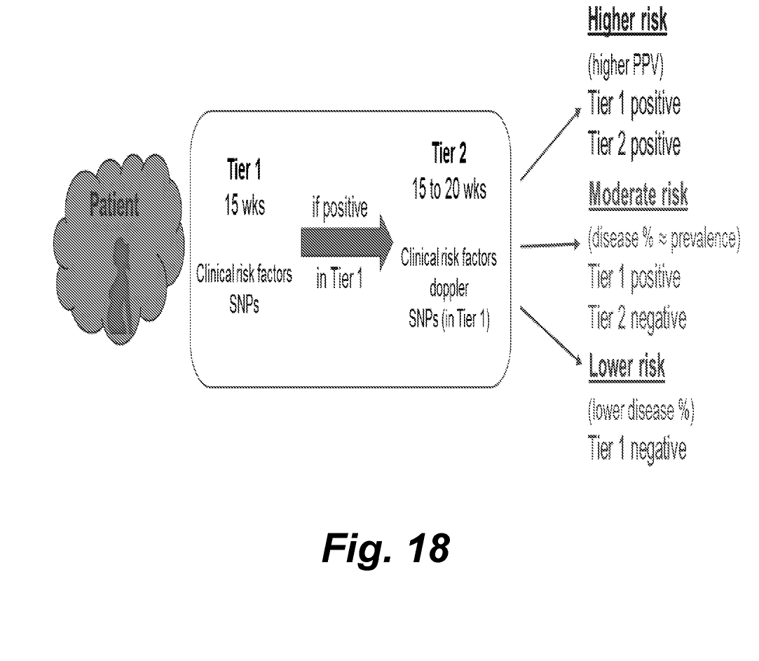

After the post-test odds for tier 2 was obtained, the predicted risk of all tiers was analyzed together and classified the risk of disease in to 3 categories: low risk, moderate risk, and high risk (FIG. 18).

Women with a negative result at tier 1 are considered as low risk, and do not need to go through further screening to tier 2. Since the sensitivity in tier 1 is high, the likelihood of disease in women who are predicted at low risk is relatively low. For women who are predicted at risk in tier 1, further screening through tier 2 is recommended to identify individuals who are at high risk. Since low-risk women are already “eliminated” in tier 1, the sensitivity threshold may be relaxed in tier 2 to aim for a higher positive predictive value. Therefore, individuals who may be at higher risk (i.e. those who have positive test result in both tier 1 and 2) may be further identified, amongst those who are predicted at risk.

As a result, the proportion of disease in the low-risk group (i.e. negative result in tier 1) will be lowest amongst the 3 risk groups, or at least lower than the current disease prevalence. Similarly, with a higher positive predictive value in the high-risk group, the proportion of disease will be highest, preferably more than 20% for rare diseases such as PE and SPTB. Hence, women with relatively lower risk are “eliminated” at each tier, and tailored care may be provided according to their classified predicted risk.

Program Code for Risk Classification

The first step in model integration was to calculate the Tier 1 and Tier 2 predicted probabilities for each patient. This is done by first retrieving the model coefficients from the two individual models, and calculate their predicted probabilities using the predict function (built-in R function). The probabilities are then dichotomized based on the chosen threshold of the corresponding tier using the cutoff function.

[R] Code Snippet 4.3.1: Workflow for predicted probabilities for each tier

cut.def <- c(0.972,0.9,0.85) # define thresholds
# load tier 1 model
M01.dat <- Mdat(M01,data)
M01.prob <- predict(M01.LR,newdata= M01.dat,type=“response”)
M01.group <- cutoff(M01.prob,cut.def[1],swap=T)
# load tier 2 model
M02.dat <- Mdat(M02,data)
M02.prob <- predict(M02.LR,newdata= M02.dat,type=“response”)
M02.group <- cutoff(M02.prob,cut.def[2],swap=T)

The Mdat function simply extracts the columns of data that are included in the fitted model.

[R] Code Snippet 4.3.2: Mdat

```
Mdat <- function(model.name,main.dat){
  var.names <-
as.matrix(read.table(paste(model.name,“_vars.txt”,sep=“”),header=F))
  cat.names <-
as.matrix(read.table(paste(model.name,“_vars_CAT.txt”,sep=“”),header=
F))
  choose.names <- var.names[1:(length(var.names)−1)]
  sub.dat <- data.choose(main.dat,c(“regid”,var.names),cat.names)
  return(sub.dat)
}
```

The cutoff function dichotomizes the predicted probability based on the chosen threshold (cut), and returns a vector of 0s and 1s.

[R] Code Snippet 4.3.3: cutoff

```
cutoff <- function(pred,cut,swap=FALSE){
  if(swap==TRUE){cut <- 1−cut}
  pred[pred<cut] <- 0
  pred[pred>=cut] <- 1
  return(pred)
}
```

After the predicted probabilities of both tiers are obtained, the positive and negative likelihood ratios of Tier 2 should be calculated. Since the likelihood ratios are obtained from the sensitivity and specificity of the model, the Xtab.simp function is used to construct 2×2 matrix of the observed cases vs. predicted cases. This is a simplified version of the Xtab function (Code Snippet 3.3.4), in which it only reads two dichotomized vectors.

[R] Code Snippet 4.3.4: Xtab.simp

```
Xtab.simp <- function(grp,out,swap=FALSE){
  Xtab.freq <- table(grp,out)
  if(swap==TRUE){
    Xtab.freq[,c(1,2)] <- Xtab.freq[,c(2,1)]
    Xtab.freq[c(1,2),] <- Xtab.freq[c(2,1),]
  {
  csum <-
c(Xtab.freq[1,1]+Xtab.freq[2,1],Xtab.freq[1,2]+Xtab.freq[2,2])
  Xtab.mod1 <- rbind(Xtab.freq,“Total”=csum)
  rsum <-
c(Xtab.mod1[1,1]+Xtab.mod1[1,2],Xtab.mod1[2,1]+Xtab.mod1[2,2],
Xtab.mod1[3,1]+Xtab.mod1[3,2])
  Xtab <- cbind(Xtab.mod1,“Total”=rsum)
  dep.tab <- table(out)
  rownames(Xtab) <-
c(paste(“Predicted(”,rownames(dep.tab)[1],“)”,sep=“”),paste(“Predicted
(”,rownames(dep.tab)[2],“)”,sep=“”),“Total”)
  colnames(Xtab) <-
c(paste(“Observed(”,rownames(dep.tab)[1],“)”,sep=“”),paste(“Observed(
”,rownames(dep.tab)[2],“)”,sep=“”),“Total”)
  if(swap==TRUE){
    rownames(Xtab) <-
c(paste(“Predicted(”,rownames(dep.tab)[2],“)”,sep=“”),paste(“Predicted
(”,rownames(dep.tab)[1],“)”,sep=“”),“Total”)
    colnames(Xtab) <-
c(paste(“Observed(”,rownames(dep.tab)[2],“)”,sep=“”),paste(“Observed(
”,rownames(dep.tab)[1],“)”,sep=“”),“Total”)
  }
  return(Xtab)}
```

The sensitivity and specificity, along with the likelihood ratios can then be calculated from the 2×2 matrix using the new rs function. The likelihood ratios are defined by:

$${LR}_{+} = \frac{r}{1 - s} {LR}_{-} = \frac{1 - r}{s}$$

where r is the sensitivity and s is the specificity of the model.

[R] Code Snippet 4.3.5: rs (for tiered models)

```
rs <- function(Xtab){
  r <- Xtab[1,1]/Xtab[3,1] #sensitivity
  s <- Xtab[2,2]/Xtab[3,2] #specificity
  o <- (Xtab[1,1]+Xtab[2,2])/Xtab[3,3] #overall accuracy
  PPV <- Xtab[1,1]/Xtab[1,3] #positive predictive value
  NPV <- Xtab[2,2]/Xtab[2,3] #negative predictive value
  LRpos <- r/(1−s) #likelihood ratio of outcome given positive result
  LRneg <- (1−r)/s #likelihood ratio of outcome given negative result
  rs.tab <-
t(data.frame(“Sensitivity”=r,“Specificity”=s,“PPV”=PPV,“NPV”=NPV,
“LR+”=LRpos,“LR-”=LRneg,“Overall”=o,check.names=F))
  return(rs.tab)
}
```

The post-test odds was then calculated using the postOdds function. This function parses the prior probabilities (from Tier 1), and multiply it by the positive or negative likelihood ratio of Tier 2, based on the dichotomized vector obtained from Tier 2.

$$pOdds = \{ \begin{matrix}k = 0 \text{:} {p}_{0} {LR}_{-} \\ k = 1 \text{:} {p}_{0} {LR}_{+}\end{matrix}$$

where k is the dichotomized predicted probability of Tier 2, and p₀ is the continuous predicted probability of Tier 1.

[R] Code Snippet 4.3.6: postOdds

```
postOdds <- function(prioro,post.grp,post.LRpos,post.LRneg){
  pOdds <- prioro #Tier 1 pred preserved when value missing
  #pOdds <- NA #if do not preserve Tier 1 pred when value missing
  pOdds.neg <- prioro *post.LRneg #Tneg
  Tneg <- which(post.grp==0)
  pOdds[Tneg] <- pOdds.neg[Tneg]
  pOdds.pos <- prioro *post.LRpos #Tpos
  Tpos <- which(post.grp==1)
  pOdds[Tpos] <- pOdds.pos[Tpos]
  return(pOdds)
}
```

The workflow for obtaining the final risk classification was as follows:

[R] Code Snippet 4.3.7: Workflow for final risk classification

M02.tab <- Xtab.simp(M02.group, M02.dat$PE,swap=T) #2×2 matrix
M02.rs <- rs(M02.tab) #LR+/−
#Integrated probability
M02i.prob <- postOdds(M01.prob, M02.group, M02.rs[5], M02.rs[6])
M02i.group <- cutoff(M02i.prob,cut.def[3],swap=T) #dichotomized
#final risk classification table
M02i.tab <-
ftable(“M02i”=M02i.group,”M01”=M01.group,”Outcome”=M02.dat$PE)

The final risk classification table (M20i.tab) was structured as follows in Table 13:

TABLE 13
Final risk classification table

Observed Outcome

| Tier 1 | Tier 2 | 0 = No | 1 = Yes | |
|---|---|---|---|---|
| 0 | 0 | | | Low risk |
| | 1 | 0 | 0 | |
| 1 | 0 | | | Moderate risk |
| | 1 | | | High risk |

Final Model Performance

The model development and testing process were repeated 10 times to obtain cross validation error rates for the final risk classification. For each iteration, stratified random sampling was performed to preserve the proportion of outcome cases within SCOPE data. All models are trained with 70% data, leaving 30% for testing purposes.

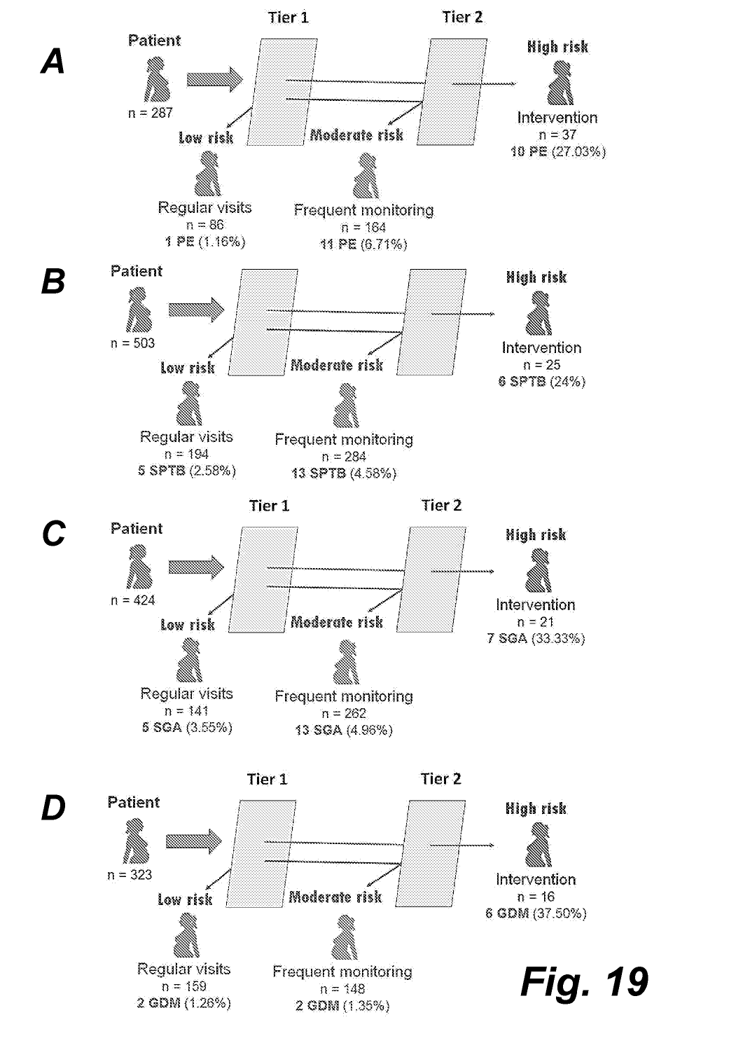

FIG. 19 shows the final risk classifications for PE (FIG. 19A), SPTB (FIG. 19B), SGA (FIG. 19C) and GDM (FIG. 19D). The rate of outcome is calculated in patients classified as low, moderate, or high risk. They are only reported on the 30% testing data.

Validation Testing

The following tables (Tables 14 to 17) are the results of all 10 repeated cross validation measures for the 30% testing data, showing the positive predictive value (PPV), negative predictive value (NPV), sensitivity of Tier 1 (r1), specificity of Tier 1 (s1), sensitivity of Tier 2 (r2), specificity of Tier 2 (s2), proportion of outcome in low risk (low), moderate risk (mod), and high risk (high) group. The average measures across all repetitions are also calculated, along with their corresponding standard deviations.

TABLE 14
10-fold cross-validation estimates for PE model

| | 1 | 2 | 3 | 4 | 5 | 6 | 7 | 8 | 9 | 10 | Average | SD |
|---|---|---|---|---|---|---|---|---|---|---|---|---|
| PPV | 0.23 | 0.24 | 0.24 | 0.21 | 0.27 | 0.20 | 0.21 | 0.20 | 0.17 | 0.21 | 0.22 | 0.03 |
| NPV | 0.97 | 0.98 | 0.98 | 0.99 | 0.99 | 0.99 | 0.99 | 1.00 | 0.98 | 1.00 | 0.99 | 0.01 |
| r1 | 0.86 | 0.91 | 0.91 | 0.95 | 0.95 | 0.95 | 0.95 | 1.00 | 0.91 | 1.00 | 0.94 | 0.04 |
| s1 | 0.33 | 0.31 | 0.33 | 0.32 | 0.32 | 0.33 | 0.32 | 0.30 | 0.34 | 0.32 | 0.32 | 0.01 |
| r2 | 0.32 | 0.50 | 0.41 | 0.14 | 0.55 | 0.41 | 0.36 | 0.41 | 0.23 | 0.55 | 0.39 | 0.13 |
| s2 | 0.91 | 0.87 | 0.89 | 0.96 | 0.88 | 0.86 | 0.88 | 0.86 | 0.91 | 0.83 | 0.89 | 0.04 |
| low | 0.03 | 0.02 | 0.02 | 0.01 | 0.01 | 0.01 | 0.01 | 0.00 | 0.02 | 0.00 | 0.01 | 0.01 |
| mod | 0.07 | 0.06 | 0.07 | 0.10 | 0.06 | 0.08 | 0.08 | 0.08 | 0.09 | 0.07 | 0.08 | 0.01 |
| high | 0.23 | 0.24 | 0.24 | 0.21 | 0.27 | 0.20 | 0.21 | 0.20 | 0.17 | 0.21 | 0.22 | 0.03 |

TABLE 15
10-fold cross-validation estimates for PTB model

| | 1 | 2 | 3 | 4 | 5 | 6 | 7 | 8 | 9 | 10 | Average | SD |
|---|---|---|---|---|---|---|---|---|---|---|---|---|
| PPV | 0.15 | 0.25 | 0.20 | 0.15 | 0.19 | 0.20 | 0.15 | 0.38 | 0.23 | 0.22 | 0.21 | 0.07 |
| NPV | 0.99 | 0.98 | 0.97 | 0.99 | 0.98 | 0.97 | 0.97 | 1.00 | 0.98 | 0.98 | 0.98 | 0.01 |
| r1 | 0.92 | 0.88 | 0.79 | 0.92 | 0.96 | 0.79 | 0.83 | 1.00 | 0.96 | 0.83 | 0.89 | 0.07 |
| s1 | 0.28 | 0.37 | 0.37 | 0.37 | 0.10 | 0.30 | 0.26 | 0.23 | 0.13 | 0.39 | 0.28 | 0.10 |
| r2 | 0.25 | 0.29 | 0.33 | 0.42 | 0.33 | 0.13 | 0.13 | 0.50 | 0.29 | 0.21 | 0.29 | 0.12 |
| s2 | 0.93 | 0.96 | 0.93 | 0.89 | 0.93 | 0.97 | 0.96 | 0.96 | 0.95 | 0.96 | 0.94 | 0.03 |
| low | 0.01 | 0.02 | 0.03 | 0.01 | 0.02 | 0.03 | 0.03 | 0.00 | 0.02 | 0.02 | 0.02 | 0.01 |
| mod | 0.05 | 0.05 | 0.04 | 0.05 | 0.04 | 0.05 | 0.05 | 0.03 | 0.04 | 0.05 | 0.04 | 0.01 |
| high | 0.15 | 0.25 | 0.20 | 0.15 | 0.19 | 0.20 | 0.15 | 0.38 | 0.23 | 0.22 | 0.21 | 0.07 |

TABLE 16
10-fold cross-validation estimates for SGA model

| | 1 | 2 | 3 | 4 | 5 | 6 | 7 | 8 | 9 | 10 | Average | SD |
|---|---|---|---|---|---|---|---|---|---|---|---|---|
| PPV | 0.17 | 0.19 | 0.25 | 0.27 | 0.43 | 0.35 | 0.32 | 0.18 | 0.20 | 0.21 | 0.26 | 0.08 |
| NPV | 1.00 | 1.00 | 0.96 | 0.97 | 0.97 | 1.00 | 0.96 | 1.00 | 0.97 | 0.97 | 0.98 | 0.02 |
| r1 | 1.00 | 1.00 | 0.92 | 0.84 | 0.96 | 1.00 | 0.92 | 1.00 | 0.72 | 0.92 | 0.93 | 0.09 |
| s1 | 0.02 | 0.00 | 0.14 | 0.31 | 0.07 | 0.01 | 0.11 | 0.06 | 0.51 | 0.16 | 0.14 | 0.16 |
| r2 | 0.16 | 0.12 | 0.16 | 0.12 | 0.24 | 0.32 | 0.24 | 0.24 | 0.12 | 0.16 | 0.19 | 0.07 |
| s2 | 0.95 | 0.97 | 0.97 | 0.98 | 0.97 | 0.96 | 0.97 | 0.93 | 0.97 | 0.96 | 0.96 | 0.01 |
| low | 0.00 | 0.00 | 0.04 | 0.03 | 0.03 | 0.00 | 0.04 | 0.00 | 0.03 | 0.03 | 0.02 | 0.02 |
| mod | 0.05 | 0.05 | 0.05 | 0.06 | 0.05 | 0.04 | 0.05 | 0.05 | 0.08 | 0.06 | 0.05 | 0.01 |
| high | 0.17 | 0.19 | 0.25 | 0.27 | 0.43 | 0.35 | 0.32 | 0.18 | 0.20 | 0.21 | 0.26 | 0.08 |

TABLE 17
10-fold cross-validation estimates for GDM model

| | 1 | 2 | 3 | 4 | 5 | 6 | 7 | 8 | 9 | 10 | Average | SD |
|---|---|---|---|---|---|---|---|---|---|---|---|---|
| PPV | 0.18 | 0.14 | 0.46 | 0.20 | 0.23 | 0.20 | 0.33 | 0.33 | 0.23 | 0.38 | 0.27 | 0.10 |
| NPV | 0.99 | 0.98 | 0.99 | 0.99 | 0.99 | 0.99 | 0.99 | 0.99 | 0.99 | 0.99 | 0.99 | 0.00 |
| r1 | 0.70 | 0.40 | 0.90 | 0.60 | 0.70 | 0.80 | 0.60 | 0.70 | 0.70 | 0.60 | 0.67 | 0.13 |
| s1 | 0.92 | 0.87 | 0.28 | 0.92 | 0.92 | 0.69 | 0.85 | 0.83 | 0.89 | 0.93 | 0.81 | 0.20 |
| r2 | 0.50 | 0.40 | 0.60 | 0.50 | 0.70 | 0.60 | 0.40 | 0.70 | 0.70 | 0.60 | 0.57 | 0.12 |
| s2 | 0.93 | 0.92 | 0.98 | 0.94 | 0.92 | 0.92 | 0.97 | 0.96 | 0.92 | 0.97 | 0.94 | 0.02 |
| low | 0.01 | 0.02 | 0.01 | 0.01 | 0.01 | 0.01 | 0.01 | 0.01 | 0.01 | 0.01 | 0.01 | 0.00 |
| mod | 0.40 | 0.00 | 0.01 | 0.14 | 0.00 | 0.03 | 0.05 | 0.00 | 0.00 | 0.00 | 0.07 | 0.13 |
| high | 0.18 | 0.14 | 0.46 | 0.20 | 0.23 | 0.20 | 0.33 | 0.33 | 0.23 | 0.38 | 0.27 | 0.10 |

User Interface

The current user interface was a Java applet. A web-based application may also be used.

System Requirements

A browser that supports Java V1.4.2 or later is required, e.g. Internet Explorer 8, Netscape 7.x, Mozilla 1.x, or later.

Java Runtime Environment 1.2.2 or later

Program Structure

Preeclampsia Calculator 0.9.8 consists of four classes: PEdrive, LRcalc, PreeclampsiaLR4 and buttonChoose. This section discusses each of these classes in detail.

Main

PEdrive contains the main class of Preeclampsia Calculator 0.9.8. It calls a new class object from PreeclampsiaLR4. Basically, PEdrive loads the program.

Code Snippet 5.2.1: PEdrive

```
public class PEdrive {
              public static void main(String[ ] args) {
              new PreeclampsiaLR4( );
    }
}
```

Prediction Algorithm Method

LRcalc contains the prediction algorithm method. It parses a double value obtained from the prediction models developed, and returns a predicted percentage.

Code Snippet 5.2.2: LRcalc

```
public class LRcalc {
        double predp;
    public double LRcalc (double formula) {
        double test1e = Math.exp(formula);
                double test1ep = 1 + test1e;
                predp = test1e/test1ep;
                return predp;
    }
}
```

Input Selection

buttonChoose contains three methods that are called in PreeclampsiaLR4. The purpose of this class is to filter and process user input data.

chkSelect

This method parses a JCheckBox and a double value which contains the coefficient of a certain predictor, and returns a double value. If the checkbox state is “Selected”, then it returns the coefficient of the predictor, otherwise, it returns 0.

Code Snippet 5.2.3: chkSelect

public double chkSelect(JCheckBox chk,double coef){

if(chk.isSelected( )){choice = coef;}
else{choice=0;}
return choice;

}

fieldSelect

This method parses a JFormattedTextField and a double value which contains the coefficient of a certain predictor, and returns a double value. This method reads the input values of the number fields and multiply it with the coefficient of the corresponding predictor.

Code Snippet 5.2.4: fieldSelect

public double fieldSelect(JFormattedTextField field,double coef){

double choosen = ((Number)field.getValue( )).doubleValue( );
choice = choosen*coef;
return choice;

}

comboSelect3

This method parses a JComboBox and two double values which contains the coefficient of certain predictors, and returns a double value. This method first obtains the selected index of the drop-down selection boxes, and chooses the corresponding coefficient of the selected item.

Code Snippet 5.2.5: comboSelect3

public double comboSelect3(JComboBox combo,double c2,double c3){

int choosen = combo.getSelectedIndex( );
if(choosen == 0){choice = 0;}
else if(choosen == 1){choice = c2;}
else if(choosen == 2){choice = c3;}
return choice;

}

Variable Definitions

PreeclampsiaLR4 contains all the user input fields, variable definitions and the GUI structure.

Predictor Variables

All predictor variables are defined as private double to account for decimal input values.

Code Snippet 5.2.6: Predictor variables

private double predp;
private double test1;

.
.
.

private double f10c_cig_1st_vst_gp;

Gap Dimensions

There are 4 default gaps in the GUI: FIELD_GAP, BUTTON_GAP, BORDER_GAP and NUMBER_GAP. FIELD_GAP is the horizontal gap between text-field label and text-field box, BUTTON_GAP is the vertical gap between buttons, BORDER_GAP is the border space, BUTTON_GAP is the default significance figure space to be displayed in number fields.

Code Snippet 5.2.7: Gap dimensions

final Dimension FIELD_GAP = new Dimension(5,0);
final Dimension BUTTON_GAP = new Dimension(0,10);
final Dimension BORDER_GAP = new Dimension(10,0);
private int NUMBER_GAP = 5;

Colours

Four colours are used in the GUI: azure, darkblue, lightgreen and greenyellow. Azure is the colour of mainPanel, darkblue is the colour of outcome text, lightgreen is the background colour of combo-boxes, and greenyellow is the colour of “Calculate” and “Clear” buttons.

Code Snippet 5.2.8: Colours

Color azure = new Color (0xf0ffff);
Color darkblue = new Color (0x00008b);
Color lightgreen = new Color (0x00ff88);
Color greenyellow = new Color (0xadff2f);

Buttons, Check Box, Text Field, and Combo Box

All buttons, checkboxes, text-fields and combo-boxes are private. Each text-field and combo-box needs a label, they are defined using JLabel.

Code Snippet 5.2.9: Buttons, check box, text field, and combo box

private JButton calc = new JButton(“Calculate”);

.
.
.

private JCheckBox f23c_20w_abn_umbriT = new
JCheckBox(“Abnormal 20 weeks doppler(umbri)”);

.
.
.

private JLabel field1 = new JLabel(“2nd MAP on 1st visit:”);
private JFormattedTextField f11c_1st_vst_map_2ndT = new

JFormattedTextField(new Double(0));

.
.
.

static final String cig[ ] = {“None”,“1-5”,“6-10”,“>10”};
private JLabel combobox1 = new JLabel(“Cigarettes on 1st visit”);
private JComboBox f10c_cig_1st_vst_gpT = new JComboBox(cig);

GUI

Box Layout is used in Preeclampsia Calculator 0.9.8. It contains 6 types of panels: mainPanel, buttonPanel, outcomePanel, chkPanel, fieldPanel and comboboxPanel. buttonPanel contains all the buttons, outcomePanel contains the outcome text, chkPanel contains the check boxes, fieldPanel contains the text-fields, comboboxPanel contains the combo-boxes, and mainPanel is the main panel which contains all the panels mentioned above.

The number of chkPanel, fieldPanel and comboboxPanel depends on the number of predictors in the model. One panel represents each row, and each row has two predictor inputs. In Preeclampsia Calculator 0.9.8, there are two chkPanel, two fieldPanel and eight comboboxPanel.

A Horizontal Glue is added between each predictor inputs within each panel. A Vertical Glue is added between each panel within the main panel. These are added to provide flexibility to relocate the panels when the window is stretched or resized.

Add Panels, Check Box, Text Field, and Combo Boxes

The panels, check-boxes, text-fields and combo-boxes are added using the methods defined below.

addPanel

This method contains the pre-defined GUI format for a panel. It first sets up a Box Layout to line up in the X-axis (i.e. horizontally), and then sets the background to transparent. Note that all panel backgrounds are set to transparent except the Main Panel. Then, a border gap is added to ensure there is a space between the border and the input fields.

Code Snippet 5.3.1: addPanel

public void addPanel(JPanel pan){

pan.setLayout(new BoxLayout(pan, BoxLayout.X_AXIS));
pan.setOpaque(false);
pan.add(Box.createRigidArea(BORDER_GAP));

}

addChk

This method contains the pre-defined GUI format for a check box. It first sets the background as transparent, and then add the checkbox to the panel with a Horizontal Glue to ensure there is a space between the two input fields within the panel.

Code Snippet 5.3.2: addChk

public void addChk(JCheckBox T, JPanel pan){

T.setOpaque(false);
pan.add(T);
pan.add(Box.createHorizontalGlue( ));

}

addField

This method contains the pre-defined GUI format for a number field. It first gets the size of the number field, and then adds the label. A FIELD_GAP is then added to ensure there is a space between the label and the number field. Before the number field is added, this method sets up the length of the number field (i.e. NUMBER_GAP) to configure the significant figures for display. After the number field is added, a Horizontal Glue is also added to ensure there is a space between the two number fields within the panel.

Code Snippet 5.3.3: addField

public void addField(JFormattedTextField field,JLabel label,JPanel

pan){

field.setMaximumSize(field.getPreferredSize( ));
pan.add(label);
pan.add(Box.createRigidArea(FIELD_GAP));
field.setColumns(NUMBER_GAP);
pan.add(field);
pan.add(Box.createHorizontalGlue( ));

}

addComboBox

This method contains the pre-defined GUI format for a combo-box. It first adds the label to the panel, and then gets the size of the combo-box. The background is set to lightgreen before it is added to the panel. Then a Horizontal Glue is added to ensure there is a space between the two combo-boxes within the panel.

Action Listener

The “Calculate” and “Clear” buttons are implemented using Action Listener. Both Action Listener alters the outcome text in outcomePanel, which displays the predicted output from the model based on the input values.

calcListener

This Action Listener first calls a new class object from buttonChoose discussed previously, which defines all the input selection methods.

Code Snippet 5.3.4: calcListener

buttonChoose choosen = new buttonChoose( );

Then, it gets all the predictor coefficients from the models developed based on the input values or selections.

Code Snippet 5.3.5: calcListener (continue)

f23c_20w_abn_umbri =
choosen.chkSelect(f23c_20w_abn_umbriT,3.602);

.
.
.

f11c_1st_vst_map_2nd =

choosen.fieldSelect(f11c_1st_vst_map_2ndT,0.3644);

.
.
.

Claire_Neo_IL6_RS1800795 =

choosen.comboSelect3(Claire_Neo_IL6_RS1800795T,21.69,19.78)

;

After all predictor coefficients are obtained, the Action Listener calculates the odds for the predicted values. Then, it calls a new class object from LRcalc as previously discussed, to obtain the predicted probability.

The outcome text alters based on the predicted probability and the cutoff value. A very basic invalid or missing value check is also performed in this Action Listener.

Code Snippet 5.3.6: calcListener (continue)

LRcalc mycalc = new LRcalc( );
predp = mycalc.LRcalc(test1);
if(f11_bmi==5 || f11c_1st_vst_map_2nd==0 || VitaminD==0 ||

f1_age==0){

outcome.setForeground(Color.RED);
outcome.setText(“INVLAID/MISSING DATA”);

}
else{

if(predp >= (1-0.85)){outcome.setText(“Pre-eclampsia”);}
else{outcome.setForeground(darkblue);outcome.setText(“NO
Pre-eclampsia”);}

}

clearListener

The “Clear” button resets all check-boxes, number fields, combo-boxes and prediction outcome. Basically, this Action Listener resets all variables, to default values.

Code Snippet 5.3.7: clearListener

f10c_cig_1st_vst_gp=0;

.
.
.

Steve_Neo_IGF2_RS680=0;

.
.
.

f23c_20w_abn_aveutriT.setSelected(false);

.
.
.

f11c_1st_vst_map_2ndT.setValue(new Double(0));

.
.
.

Steve_Neo_IGF2_RS680T.setSelectedIndex(0);

.
.
.

test1 = 0;
outcome.setForeground(darkblue);
outcome.setText(“Press Calculate”);

WindowListener

An addition Listener is added to Preeclampsia Calculator 0.9.8. This Listener makes the applet “exit on close”.

Code Snippet 5.3.8: WindowListener

class PreeclampsiaLR4WindowListener extends WindowAdapter {

public void windowClosing(WindowEvent event){

System.exit(0);

}

### Example 4—Pregnancy Calculators

Introduction

Pregnancy Calculator is a set of four calculators that predicts possible Preeclampsia, Preterm birth, Small for Gestational Age, and Gestational Diabetes Mellitus cases using potential predictors obtained from statistical analysis on the SCOPE database.

Pre-Requisites

A browser that supports Java V1.4.2 or later is required, e.g. Internet Explorer 8, Netscape 7.x, Mozilla 1.x, or later.

Java Runtime Environment 1.2.2 or Later

Using Pregnancy Calculators

Opening Calculators

Pregnancy Calculators (alpha) can be used as an embedded Java Applet within a web page, which can be viewed using a web browser. A compiled and executable jar file is also available. No installation is required.

Data Input Fields

Each of the calculators contains three input fields:
Checkboxes (Select if YES)
Number fields
Drop-down Selections
An “INVALID/MISSING DATA” message is displayed when predictor values are missing or invalid.

Calculate/Clear

When all predictor values are inserted, press “Calculate”. The predicted outcome will be displayed at the bottom. Press “Clear” to reset the calculator.

### Example 5—Preeclampsia Calculator

Preeclampsia Calculator

Preeclampsia Calculator requires the following inputs:

5 Checkboxes (Select if YES)
Family history of Preeclampsia
Family history of chronic hypertension
Any previous miscarriage (≤10 weeks) with same partner
≥12 months to conceive
Any vaginal bleeding ≥5 days

6 Number fields
Patient age
Patient BMI
Mean Arterial Pressure
Patient's birth weight
Units of alcohol consumed per week during first trimester
Number of cigarettes per day at 15 weeks' gestation

14 Drop-down selections
Frequency of fruit consumption 1 month prior to pregnancy
Maternal AGTR1 (rs5186)
Maternal IL10 (rs1800896)
Maternal MTHFR (rs1801131)
Maternal PGF (rs1042886)
Maternal PLG (rs2859879)
Maternal INSR (rs2059806)
Paternal NOS2A (rs1137933)
Paternal TP53 (rs1042522)
Paternal MTHFR (rs1800469)
Paternal INS (rs3842752)
Paternal TFGB (rs1800469)
Paternal PFG (rs1042886)
Paternal MMP2 (rs243865)

Case 1

The patient, aged 27, has not smoked any cigarettes at 15 weeks' gestation, with no family history of Preeclampsia or chronic hypertension. She has no miscarriage with same partner, no vaginal bleeding of 5 days or more, and did not take more than 12 months to conceive. Her BMI is 24.8, with a MAP of 73, and birth weight of 3800 g. She eats more than 1 fruit per day one month prior pregnancy and had consumed 2 units of alcohol per week during first trimester.

With the use of the above indicated SNPs, she would be considered as low risk for Preeclampsia.

Case 2

A patient with the following characteristics would be predicted as moderate risk for Preeclampsia.

The patient, aged 35, has not smoked any cigarettes at 15 weeks' gestation, with a family history of Preeclampsia and chronic hypertension. She has no miscarriage with same partner, no vaginal bleeding of 5 days or more, and did not take more than 12 months to conceive. Her BMI is 22.1, with a MAP of 75, and birth weight of 3203 g. She eats 1-3 fruit per month one month prior pregnancy and had consumed 0 units of alcohol per week during first trimester.

With the use of the above indicated SNPs, she would be considered as moderate risk for Preeclampsia.

Case 3

A patient with the following characteristics would be predicted at high risk for Preeclampsia.

The patient, aged 32, has not smoked any cigarettes at 15 weeks' gestation, has a family history of Preeclampsia and chronic hypertension. She has no miscarriage with same partner, no vaginal bleeding of 5 days or more, and did take more than 12 months to conceive. Her BMI is 32, with a MAP of 85, and birth weight of 2,610 g. She eats 1 or more fruit per day one month prior pregnancy and had consumed 0 units of alcohol per week during first trimester.

With the use of the above indicated SNPs, she would be considered as high risk for Preeclampsia.

### Example 6—Preterm Birth Calculator

Preterm Birth Calculator requires the following inputs:

7 Checkboxes (Select if YES)
Family history of low-birth weight baby
Family history of spontaneous preterm birth
Used marijuana >90 times (more than once per day) during first trimester
Consumed/inhaled/injected other recreational drugs
Score for State-Trait Anxiety Inventory >90^{th} centile
On metformin for PCOS prior to or at conception
Any hospital admissions due to hyperemesis

6 Number fields
Patient BMI
Patient height (cm)
Gravidity
Months to conceive
Years of schooling
Transvaginal cervical length (mm) at 20 weeks' gestation

20 Drop-down selections
Number of times participant's mother had Preeclampsia
Folic Acid (μg per day) during 1^{st} trimester
Number of times climbed stairs in the last month
Maternal BCL2 (rs2279115)
Maternal TCN2 (rs1801198)
Maternal IGF2R (rs2274849)
Maternal uPA (rs2227564)
Maternal MMP2 (rs243865)
Maternal TIMP3 (rs5749511)
Number of LLETZ treatment
Household members
Feeling better than ever in pregnancy
Frequency of fruit consumption month prior to pregnancy
Maternal ADD1 (rs4961)
Maternal MBL2 (rs1800450)
Maternal FLT1 (FLT1C677T)
Maternal IL1B (rs16944)
Maternal IGF1R (rs11247361)
Maternal MMP9 (rs3918242)

Case 1

For example, a patient with the following characteristics would be considered as low risk for preterm birth.

The patient has no family history of a low birth weight baby, no family history of spontaneous pre term birth, no use of marijuana greater than >90 times in 3 months, no use of other recreation drugs, no state-trait anxiety inventory >90^{th} centile, is not on metformin for PCOS prior to or at conception, has no hospital admissions due to hyperemesis, a BMI of 22.7, a height of 176 cm, a gravidity of 1, took 6 months to conceive, has 12 years of schooling, a transvaginal cervical length of 32 mm, the patient's mother did not have preeclampsia, no LLETZ treatments, no stairs climbed in the last month, folic acid of less than or equal to 800 μg, a single household partner, rarely felt better than ever in pregnancy, and ate 1 or more fruits per day.

With the use of the above indicated SNPs, she would be considered as low risk for preterm birth.

Case 2

For example, a patient with the following characteristics would be considered as moderate risk for preterm birth.

The patient has no family history of a low birth weight baby, no family history of spontaneous pre term birth, no use of marijuana greater than >90 times in 3 months, no use of other recreation drugs, no state-trait anxiety inventory >90^{th} centile, is not on metformin for PCOS prior to or at conception, has no hospital admissions due to hyperemesis, a BMI of 35.4, a height of 170 cm, a gravidity of 2, took 4 months to conceive, has 12 years of schooling, a transvaginal cervical length of 42 mm, the patient's mother did not have preeclampsia, no LLETZ treatments, no stairs climbed in the last month, folic acid of less than or equal to 800 μg, a single household partner, on some days felt better than ever in pregnancy, and ate 1 or more fruits per month.

With the use of the above indicated SNPs, she would be considered as moderate risk for preterm birth.

Case 3

For example, a patient with the following characteristics would be considered as high risk for Preterm birth.

The patient has no family history of a low birth weight baby, no family history of spontaneous pre term birth, no use of marijuana greater than >90 times in 3 months, no use of other recreation drugs, has a state-trait anxiety inventory >90^{th} centile, is not on metformin for PCOS prior to or at conception, has no hospital admissions due to hyperemesis, a BMI of 28, a height of 158 cm, a gravidity of 2, took 1 month to conceive, has 12 years of schooling, a transvaginal cervical length of 48 mm, the patient's mother did not have preeclampsia, no LLETZ treatments, no stairs climbed in the last month, folic acid of less than or equal to 800 μg, a household member who are parents, on some days felt better than ever in pregnancy, and ate 1 or more fruits per week.

With the use of the above indicated SNPs, she would be considered as high risk for preterm birth.

### Example 7—Small for Gestational Age Calculator

Small for Gestational Age Calculator requires the following inputs:

7 Checkboxes (Select if YES)
Family history of chronic hypertension
≥2 family members had delivered a baby preterm
Other ethnicity (NOT any of below):
   Caucasian
   Maori
   Aboriginal
   Polynesian
   Melanesian
   Micronesia
   South East Asian
   Chinese
   Far East Asia
   Indian
   African ancestry
   Afro-Caribbean
   Middle-eastern
   Hispanic
   South American
Consumed/inhaled/injected other recreational drugs
Use of barrier contraception (condoms or diaphragm) with biological father of baby
Snoring most nights
Any computer usage in last month

5 Number fields
Patient BMI
Mean Arterial Pressure
Patient's head circumference (cm)
During of light vaginal bleeding at or before 6 weeks' gestation
Hours worked in paid employment per week at 15 weeks' gestation

10 Drop-down selections
Smoking status
Patient's rhesus factor (positive or negative)
Partner's paid work status
Maternal IL6 (rs1800795)
Maternal F2 (rs1799963)
Maternal INS (rs3842752)
Paternal TCN2 (rs18001198)
Paternal THBS1 (rs2228262)
Paternal IGF2 (rs3741204)
Paternal IGF2AS (rs1004446)

Case 1

For example, a patient with the following characteristics would be considered as low risk for Small for Gestational Age.

The patient has no family history of chronic hypertension, no family members that have delivered a baby preterm, of no other ethnicity, no use of other recreation drugs, has used barrier contraception, does not snore most nights, has had computer usage in the last month, a BMI of 21.5, a MAP at weeks of 65, a head circumference of 55 cm, has had no light vaginal bleeding at/before 6 weeks, has 10 hours worked of paid employment per week, has a smoking status of smoking at 15 weeks, has a rhesus factor of Rh Negative, and the partner does not have paid work.

With the use of the above indicated SNPs, she would be considered as low risk for SGA.

Case 2

For example, a patient with the following characteristics would be considered as moderate risk for Small for Gestational Age.

The patient has a family history of chronic hypertension, no family members that have delivered a baby preterm, of no other ethnicity, no use of other recreation drugs, has not used barrier contraception, does not snore most nights, has had computer usage in the last month, a BMI of 24.4, a MAP at weeks of 79, a head circumference of 53 cm, has had no light vaginal bleeding at/before 6 weeks, has 10 hours worked of paid employment per week, has a smoking status of never smoked, has a rhesus factor of Rh Positive, and the partner has full time paid work.

With the use of the above indicated SNPs, she would be considered as moderate risk for SGA.

Case 3

For example, a patient with the following characteristics would be considered as high risk for Small for Gestational Age.

The patient has no family history of chronic hypertension, no family members that have delivered a baby preterm, of no other ethnicity, no use of other recreation drugs, has used barrier contraception, does not snore most nights, has had computer usage in the last month, a BMI of 19.9, a MAP at weeks of 83, a head circumference of 52 cm, has had no light vaginal bleeding at/before 6 weeks, has 40 hours worked of paid employment per week, has a smoking status of smoking at 15 weeks, has a rhesus factor of Rh Positive, and the partner has full time paid work.

With the use of the above indicated SNPs, she would be considered as high risk for SGA.

### Example 8—Gestational Diabetes Mellitus Calculator

Gestational Diabetes Mellitus Calculator requires the following inputs:

3 Checkboxes (Select if YES)
Any previous terminations at >10 weeks
Patient's father has type 2 diabetes
Consumed fruit ≥3 times per day at 1 months prior to pregnancy

6 Number fields
Patient BMI
Months to conceive
Diastolic blood pressure
Pulse per minute
Random glucose (mmol/L)
Folic Acid (μg per day) during first trimester

4 Drop-down selections
Maternal NOS2A (rs1137933)
Maternal XRCC2 (rs3218536)
Maternal KDR (rs2071559)
Maternal CYP11A (rs8039957)

Case 1

For example, a patient with the following characteristics would be considered as low risk for Gestational Diabetes Mellitus.

The patient has not had any previous termination at >10 weeks, the patient's father does not have type 2 diabetes, does eat fruit greater than or equal to 3 times per day 1 month pre-pregnancy, a BMI of 30.6, took 5 months to conceive, has a diastolic BP (15 weeks) of 80, has a pulse per minute (15 weeks) of 62, has a random glucose (15 weeks) of 5.2, and has folic acid (μg per day) in 1^{st} trimester of 800.

With the use of the above indicated SNPs, she would be considered as low risk for GDM.

Case 2

For example, a patient with the following characteristics would be considered as moderate risk for Gestational Diabetes Mellitus.

The patient has not had any previous termination at >10 weeks, the patient's father does not have type 2 diabetes, does not eat fruit greater than or equal to 3 times per day 1 month pre-pregnancy, a BMI of 22.1, took 4 months to conceive, has a diastolic BP (15 weeks) of 58, has a pulse per minute (15 weeks) of 72, has a random glucose (15 weeks) of 7.4, and has folic acid (μg per day) in 1^{st} trimester of 300.

With the use of the above indicated SNPs, she would be considered as moderate risk for GDM.

Case 3

For example, a patient with the following characteristics would be considered as high risk for Gestational Diabetes Mellitus.

The patient has not had any previous termination at >10 weeks, the patient's father does not have type 2 diabetes, does not eat fruit greater than or equal to 3 times per day 1 month pre-pregnancy, a BMI of 39.3, took 1 months to conceive, has a diastolic BP (15 weeks) of 72, has a pulse per minute (15 weeks) of 72, has a random glucose (15 weeks) of 5.4, and has folic acid (μg per day) in 1^{st} trimester of 1,050.

With the use of the above indicated SNPs, she would be considered as high risk for GDM.

### Example 9—Recommendations for Antenatal Care Following Risk Prediction for Pregnancy Complications

For each woman determined to be at risk, preventative strategies to prevent or reduce the severity of disease may be used.

Recommendations for intervention following risk assessment using the algorithms are listed in Tables 18 to 21.

TABLE 18

| Preeclampsia | Care provider | Management |
|---|---|---|
| Low risk | Midwife care | Current standard antenatal care for |
| | | nulliparous pregnant women are |
| | | visits at booking, after the |
| | | morphology scan i.e about 20 |
| | | weeks, 25 weeks, 28 weeks, 31, 33, |
| | | 35, 37, 38, 39 and 40 weeks. |
| | | Reduced antenatal visits - after |
| | | morphology scan at 20 weeks: 28, |
| | | 32, 35, 37, 39 weeks |
| | | Reduction of 4 visits |
| Moderate risk | Midwife care | Standard antenatal visits for |
| | | nulliparous women or women with |
| | | new paternity - after morphology + |
| | | uterine artery Doppler scan at 20 |
| | | weeks, i.e.: 24, 27, 30, 32, 34, 36, |
| | | 37, 38, 39 weeks' |
| | | If normal, manage as for low risk |
| | | If abnormal, see specialist after |
| | | results of morphology + uterine |
| | | Doppler. |
| | | If positive Doppler, test Flt1:PlGF |
| | | at 34 weeks' |
| High Risk (PPV | Specialist care | 100 mg Aspirin start as soon as |
| 26%) | | possible, and at least prior to 16 |
| | | weeks |
| | | Monthly growth scans |
| | | sFlt/PlGF ratio at 28 and 34 week's |
| | | If BMI ≥30 then also treat with |
| | | metformin |

TABLE 19

| Preterm Birth | Care provider | Management |
|---|---|---|
| Low risk | Midwife care | Reduced antenatal visits - after |
| | | morphology scan at 20 weeks: 28, |
| | | 32, 35, 37, 39 weeks |
| | | Reduction of 4 visits |
| Moderate risk | Midwife care | If cervical length at 20 weeks >25 mm, |
| | | manage as for low risk |
| | | If cervical length at 20 weeks ≤25 mm |
| | | manage as for high risk |
| High Risk | Specialist care | Vaginal progesterone 200 mg |
| (PPV 25%) | | daily dose - start following test |
| | | For women with a cervix <20 mm |
| | | consider cervical pessary |

TABLE 20

| IUGR <5^{th} centile | Care provider | Management |
|---|---|---|
| Low risk | Midwife care | Reduced antenatal visits - after |
| | | morphology scan at 20 weeks: 28, |
| | | 32, 35, 37, 39 weeks |
| | | Reduction of 4 visits |
| Moderate risk | Midwife care | Regular antenatal clinics. |
| | | Extra growth scan at 32 weeks |
| High Risk | Specialist care | 100 mg Aspirin start prior to 16 |
| (PPV 27%) | | week's |
| | | Monthly growth scans |

TABLE 21

| GDM | Care provider | Management |
|---|---|---|
| Low risk | Midwife care | Reduction of 4 visits |
| | | Oral Glucose tolerance test |
| | | (OGTT) at 28 weeks as in routine |
| | | antenatal care |
| Moderate risk | Midwife care | OGTT at 28 weeks |
| High Risk (PPV | Specialist care | OGTT in first trimester - if |
| 27%) | | diabetes is diagnosed - treatment |
| | | with Metformin and/or insulin |
| | | In case of normal first trimester |
| | | OGTT, OGTT at 28 weeks |
| | | Treat as appropriate under |
| | | obstetrician care. |
| | | Pending results of GROW study |
| | | these patients may be offered |
| | | Metformin from early pregnancy |

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1. A method of determining the risk of a complication of pregnancy occurring in a subject, the pregnancy arising in the subject from a conception from a maternal donor and a paternal donor, the method comprising:
receiving initial information comprising (i) initial genetic information and/or (ii) initial clinical information and/or (iii) initial lifestyle information, the initial information being from the subject and/or the maternal donor and/or the paternal donor;
processing the initial information to classify the risk of a complication of pregnancy occurring in the subject as low risk or increased risk;
for the subject having said increased risk, receiving further information, the further information comprising (i) further genetic information and/or (ii) further clinical information and/or (iii) further lifestyle information from the subject and/or the maternal donor and/or the paternal donor;
processing the further information to classify the risk in the subject having said increased risk as moderate risk or high risk;
thereby determining the risk of a complication of pregnancy occurring in the subject.

2.-4. (canceled)

5. The method according to claim 1, wherein the complication of pregnancy comprises preeclampsia, preterm birth, small for gestational age or gestational diabetes mellitus.

6-9. (canceled)

10. The method according to claim 1, wherein the initial genetic information and/or further genetic information comprise allelic information and/or DNA methylation information.

11. The method according to claim 1, wherein the initial genetic information and/or further genetic information comprises information relating to the presence and/or absence of one or more polymorphisms.

12-14. (canceled)

15. The method according to claim 1, wherein the classifying of the initial information and/or the classifying of the further information comprises penalized logistic regression.

16. The method according to claim 1, wherein the classifying of the initial information and/or classifying of the further information comprises classifying the risk on the basis of a selected probability threshold.

17. The method according to claim 1, wherein the classifying of the further information comprises classifying the risk on the basis of a selected probability threshold calculated from the initial information.

18-19. (canceled)

20. The method according to claim 5, wherein the complication of pregnancy comprises preeclampsia and the initial genetic information and/or further genetic information comprises genetic information from one or more of maternal AGT, maternal AGTR1, maternal IL10, paternal HIF1a, paternal MTRR, maternal MTHFR, maternal TGFB, maternal PGF, maternal PLG, maternal INSR, paternal NOS2A, paternal TP53, paternal MTHFR, paternal GSTP1, paternal INS, paternal TGFB, maternal PGF, paternal PGF, paternal CYP11A1, maternal INSR, and paternal MMP2.

21-24. (canceled)

25. The method according to claim 5, wherein the complication of pregnancy comprises preterm birth and the initial genetic information and/or further genetic information comprises genetic information from one or more of maternal AGT, maternal BCL2, maternal TCN2, maternal IGF2R, maternal uPA, maternal MMP2, maternal TIMP3, maternal ADD1, maternal MBL2, maternal FLT1, maternal IL1B, maternal IGF1R, maternal MMP9, and maternal CYP11A1.

26-29. (canceled)

30. The method according to claim 5, wherein the complication of pregnancy comprises small for gestational age and the initial genetic information and/or further genetic information comprises genetic information from one or more of maternal IL6, maternal F2, maternal NAT1, paternal NAT1, maternal INS, paternal TCN2, paternal THBS1, paternal IGF2, and paternal IGF2AS.

31-34. (canceled)

35. The method according to claim 5, wherein the complication of pregnancy comprises gestational diabetes mellitus and the initial genetic information and/or further genetic information comprises genetic information from one or more of maternal AGT, maternal FTO, maternal NOS2A, maternal PTEN, maternal CYP24A1, maternal XRCC2, maternal ANGPT1, maternal KDR, maternal CYP11A, and maternal H19.

36-37. (canceled)

38. The method according to claim 1, wherein the method comprises obtaining a sample from the maternal donor and/or the paternal donor and processing the sample to obtain the initial genetic information and/or further genetic information.

39. The method according to claim 1, wherein the method comprises using a computer processor means.

40. The method according to claim 39, wherein the computer processor means is used to classify the initial information and/or the further information.

41. The method according to claim 39, wherein the initial information and/or further information is received from at least one user device in data communication with the computer processor means over a network.

42. A method of determining the risk of a complication of pregnancy occurring in a subject, the pregnancy arising in the subject from a conception from a maternal donor and a paternal donor, the method comprising using a computer processor means to:
receive initial information comprising (i) initial genetic information and/or (ii) initial clinical information and/or (iii) initial lifestyle information, the initial information being from the subject and/or the maternal donor and/or the paternal donor, and process the initial information to classify the risk of a complication of pregnancy occurring in the subject as low risk or increased risk;
for the subject having said increased risk, receive further information, the further information comprising (i) further genetic information and/or (ii) further clinical information and/or (iii) further lifestyle information from the subject and/or the maternal donor and/or the paternal donor; and
process the further information to classify the risk in the subject having said increased risk as moderate risk or high risk; and
output the risk of the complication of pregnancy occurring in the subject.

43. A method of preventing and/or treating a complication of pregnancy in a subject, the method comprising using a method according to claim 1 to determine the risk of a complication of a pregnancy occurring and treating the subject on the basis of the risk so determined.

44. The method according to claim 43, wherein the complication of pregnancy comprises pre-eclampsia and the method comprises treating a subject at high risk with low dose aspirin.

45-46. (canceled)

47. A system for determining the risk of a complication of pregnancy occurring in a subject, the pregnancy arising in the subject from a conception from a maternal donor and a paternal donor, the system comprising a computer processor configured to:
receive initial information from at least one user device in data communication with the processor over a network, the initial information comprising (i) initial genetic information and/or (ii) initial clinical information and/or (iii) initial lifestyle information, the initial information being from the subject and/or maternal donor and/or the paternal donor, and
process the initial information to classify the risk of a complication of pregnancy occurring in the subject as low risk or increased risk;
for the subject having said increased risk, the processor is further configured to receive further information, from the at least one user device or a further user device in data communication with the processor, the further information comprising (i) further genetic information and/or (ii) further clinical information and/or (iii) further lifestyle information from the subject and/or the maternal donor and/or the paternal donor, and process the further information to classify the risk in the subject having said increased risk as moderate risk or high risk; and
output the risk of the complication of pregnancy occurring in the subject.

48. (canceled)

49. Computer software encoded with programming instructions executable by a computer processor means to allow the computer processor means to determine the risk of a complication of pregnancy occurring in a subject, the pregnancy arising in the subject from a conception from a maternal donor and a paternal donor, wherein the software allows the computer processing means to:
receive initial information comprising (i) initial genetic information and/or (ii) initial clinical information and/or (iii) initial lifestyle information, the initial information being from the subject and/or the maternal donor and/or the paternal donor, and
process the initial information to classify the risk of a complication of pregnancy occurring in the subject as low risk or increased risk; and
for the subject having said increased risk, receive further information, the further information comprising (i) further genetic information and/or (ii) further clinical information and/or (iii) further lifestyle information, the further information being from the subject and/or the maternal donor and/or the paternal donor, and process the further information to classify the risk in the subject having said increased risk as moderate risk or high risk.
